(19) United States
(12) Patent Application Publication
(10) Pub. No.: US 2020/0135339 A1
(43) Pub. Date: Apr. 30, 2020

(54) SYSTEMS AND METHODS FOR EXECUTING PATHWAYS FOR HEALTHCARE

(71) Applicant: LifecareX AB, Stockholm (SE)

(72) Inventors: James N. Weinstein, Lyme, NH (US); Deborah A. Jadczak, Warner, NH (US); Ethan M. Berke, Hanover, NH (US); Justin B. Montgomery, Claremont, NH (US); McKay L. Larson, Windsor, VT (US); Meghan Poperowitz, Lebanon, NH (US); Michael J. Smyth, Orford, NH (US); Nate E. Larson, Windsor, VT (US); Vincent J. Fusca, III, Windsor, VT (US); Curtis Petersen, Lebanon, NH (US)

(73) Assignee: LifecareX AB, Stockholm (SE)

(21) Appl. No.: 16/727,588

(22) Filed: Dec. 26, 2019

Related U.S. Application Data
(63) Continuation of application No. 15/371,673, filed on Dec. 7, 2016, now abandoned.
(60) Provisional application No. 62/263,985, filed on Dec. 7, 2015.

(51) Int. Cl. G16H 50/20; G08B 21/02; G16H 40/63; G16H 10/60

(52) U.S. Cl. CPC G16H 50/20; G08B 21/02; G06F 3/0482; G16H 10/60; G16H 40/63

(57) ABSTRACT

The present application is directed to systems and methods providing a pathway configurable and executable via a pathway engine on a device. A device may establish a pathway configured via a pathway engine to execute on the device to monitor data points of a user and to generate actions based on monitoring of the data points. A monitor of the pathway engine may monitor data points of the user received by the device. The monitor may compare each value of the data points to the expected value and the predetermined threshold specified for the trigger condition. The pathway, based on the comparison, may determine that the trigger condition has been triggered. The pathway, responsive to the triggering of the trigger condition, may initiate an alert to identify the trigger condition and execution of the action specified by the pathway.

### CROSS-REFERENCE TO RELATED APPLICATIONS

This application is a continuation of, and claims the benefit of and priority to U.S. patent application Ser. No. 15/371,673, titled “SYSTEMS AND METHODS FOR EXECUTING PATHWAYS FOR HEALTHCARE,” filed Dec. 7, 2016, which claims the benefit of and priority to U.S. Provisional Patent Application No. 62/263,985, titled “SYSTEMS AND METHODS FOR FACILITATING MEDICAL CARE,” filed on Dec. 7, 2015, all of which are incorporated herein by reference in its entirety for all purposes.

### BACKGROUND

Various types of medical data regarding an individual may be collected using various sensors to provide diagnoses and treatment plans. Processing vast amounts of medical data from various sensors, however, may be difficult. As such, current techniques may overlook correlations among data points and the relevance of certain data points.

### SUMMARY

Various embodiments are directed to systems and methods for providing individualized interventions, coaching, and recommendations from live connected human support (text, phone, video chat, and electronic communication) or automated response through smart device and/or wearable communication systems. In some embodiments, live support may be informed by a system dashboard or “mission control” view that provides highly customized and individualized recommendations and decision support tools created through the utilization of custom written algorithms, evidence-based data sets, population segmentation rules, and machine learning. In some embodiments, this utility allows the live support system to resolve issues proactively and predictively with the end-user. In some embodiments, the utility can also triage or transfer the end-user to an established alternative service using “warm hand offs” or establish connections to alternative services to resolve the end-user's needs using high levels of customer support.

In some embodiments, a system provides inter-pathway contextual relevance. Live incoming data analysis may be based on best practice evidence and pathway logic which gives data meaning for consumers, clinicians, and businesses.

In some embodiments, regarding pathway functionality, while each pathway monitors a defined set of parameters and creates actions solely based on those data points, the present solution integrates each pathway to influence each other at clinically relevant points. The complex interaction of pathways with each other can be described as a single large unit or system. Each part of that system can function independently but becomes increasingly enriched with inputs from other pathways. While this concept can be thought of as an “Ecological Model” of health, this term may be usually reserved for large external factors such as social determents of health. The present solution applies the Ecological Model of health to measureable components. Compiling these data points provides a more complete view of an individual's health and wellness in the context of their life. The intercorrelation and complex interaction of these factors may be built on medical knowledge, best evidence, and experience.

In some embodiments, the function of each care pathway may be to take each disparate data point and determine its relevance in the context of an individual's complete health scope, and communicate with the customer to provide information, advice, and behavioral change support. Each pathway can be upgraded to include varying types of communication with clinical or behavioral staff and can also feed aggregated information to a customer's care team and/or a corporate population health manager.

In one embodiment, a sleep pathway may be provided. Specific sleep patterns may correlate with the progression of various diseases, worsening moods, and increasing risks. In some embodiments, a pathway logic recognizes when an individual may be experiencing these sleep patterns associated with worsening diseases such as chronic obstructive pulmonary disease (COPD), diabetes, congestive heart failure (CHF), and hypertension. In some embodiments, this same methodology may be applied to non-disease states, behaviors, and moods which drastically influence an individual's health. Accordingly, in some embodiments, contextual information that gives an individual the ability to make meaningful changes in their lives may be provided. In some embodiments, beyond surfacing a current state to a user, the present solution can support automatic guided methods for improving sleep independently and in concert with the user's current health status based on other health data.

In one embodiment, a nutrition pathway may be provided. Nutritional pathway information may be incorporated into sleep, physical activity, behavior, or mood, along with disease specific logic for congestive heart failure, diabetes, chronic obstructive pulmonary disease (“COPD”), hypertension, kidney disease, surgery prep and recovery, asthma, and cancer care. Information from the nutritional pathway may be deconstructed into micro and macro-nutritional data points along with hydration levels. The present solution may use this information in the context of other health factors, behaviors, and past use to provide feedback on a customer's consumption habits.

In one embodiment, a physical activity pathway may be provided. The activity pathway may link an individual's physical activity to behaviors, moods, nutritional inputs, disease recovery, and physiological outcomes. Variations in activity within the context of these relationships may provide an opportunity to help improve a person's health through highlighting how activity has attributed to their well-being. The physical activity pathway may build on expansive evidence that increasing activity positively influences health outcomes. In some embodiments, behavior change to guide meaningful improvement in health with increasing physical activity and meeting a customer's goals may be provided.

In one embodiment, a mood and behavior pathway may be provided. Tracking an individual's mood in the context of behaviors, external data, physical activity, sleep, nutrition, and disease progression may provide each pathway context on how a customer may be actually feeling. In some embodiments, examining mood involves scoring standardized tests and multiple passive tracking metrics, such as, but not limited to, verbal inflection patterns, geo data variation, and written word communication. These methods may be compiled to create a complete view of an individual's mood, which, correlated with a known behavior, may be used as an additional data point in each pathway.

In some embodiments, data that is not directly collected, such as weather, environmental, social media, and social economic information may also may be used as factors in determining a meaningful response to a customers' needs. This information may be considered in aggregate and singularly for an individual and defined cohorts.

In some embodiments, each disease and medical condition takes data from each of the above factors and data about other diseases that the customer might have, and responds to that aggregate information in clinically relevant ways. Relevant diseases and medical conditions include, but are not limited to, diabetes, congestive heart failure, COPD, hypertension, cancer treatment, pre- and post-surgery, asthma, kidney disease, or the like.

In some embodiments, machine learning may be implemented. While the interrelationships of each disease state or health factor may be based on current evidence and an individual's norms and standards, machine learning may provide new connections and insights on both an individual and a population level. Machine learning may function broadly over all pathways on two levels. For the individual, machine learning may help to determine unique characteristics of an individual's health. At the population level, machine learning may assess large patterns and correlations that can help to inform how each pathway should communicate with each customer based on the customers' data.

In at least one aspect, the present disclosure is directed to a method of providing a pathway configurable and executable via a pathway engine on a device. A device may establish a pathway configured via a pathway engine to execute on the device to monitor one or more data points of a user and to generate one or more actions based on monitoring of the one or more data points. The device may receive a specification for the pathway of a data point to be monitored, an expected value of the data point, a trigger condition using a comparison of a value of the data point to be monitored with the expected value to a predetermined threshold, and an alert and an action to take as a result of triggering the trigger condition. A monitor of the pathway engine may monitor a plurality of data points of the user received by the device. The monitor may compare each value of the plurality of data points to the expected value and the predetermined threshold specified for the trigger condition. The pathway, based on the comparison, may determine that the trigger condition has been triggered. The pathway, responsive to the triggering of the trigger condition, may initiate an alert to identify the trigger condition and execution of the action specified by the pathway.

In some embodiments, initiating the alert to identify the trigger condition and execution of the action specified by the pathway may further comprise determining that a difference between the value of the data point and the expected value may be greater than the predetermined threshold. In some embodiments, monitoring the plurality of data points of the user received by the device may further comprise monitoring the data point by receiving data from a sensor measuring the value of the data point. In some embodiments, the action may comprise actuating an actuator coupled to the user.

In some embodiments, the device may adjust the expected value of the data point based on the action, responsive to executing the action. In some embodiments, the device may adjust an expected value of a second data point of the pathway based on the action, the second data point of the user identified in the specification for the pathway as dependent on the first data point and the action, responsive to executing the action.

In some embodiments, executing the action may further comprise selecting, from a plurality of candidate actions, the action for execution. In some embodiments, selecting, from the plurality of candidate actions, the action for execution may further comprise determining, from a client device of the user, contextual information pertaining to the user via one or more monitors of the pathway engine. In some embodiments, selecting, from the plurality of candidate actions, the action for execution may further comprise selecting, using the contextual information, the action for execution from the plurality of candidate actions. In some embodiments, the contextual information may include location information from the client device of the user. In some embodiments, the contextual information may include a plurality of physiological measurements.

In at least one aspect, the present disclosure is directed to a method of providing a multi-level pathway configurable and executable via a pathway engine on a device. The device may establish a pathway configured via a pathway engine to execute on the device to monitor one or more data points of a user and trigger cascading levels of actions based on monitoring of the one or more data points. The device may receive a specification for the pathway of a data point to be monitored, an expected value of the data point, a trigger condition using a comparison of a value of the data point to be monitored with the expected value to a predetermined threshold, and a first level alert and a first action to take as a result of triggering the triggering condition. The device may receive, for configuration of the pathway, one of a condition of the user or frequency of triggering the trigger condition upon which to trigger a second level alert and a second action to take subsequent to the triggering of the first level alert and the first action. A monitor of the pathway engine may compare values of a plurality of data points received by the device to the expected value and the predetermined threshold specified for the trigger condition. The pathway may initiate, responsive to the triggering of the trigger condition, the first level alert to identify the trigger condition and execution of the first action specified by the pathway. The monitor may monitor, responsive to the triggering of the trigger condition, values of the plurality of data points for one of the conditions of the user or frequency of triggering the trigger condition upon which to trigger the second level alert and the second action.

In some embodiments, initiating responsive to the triggering of the trigger condition, the first level alert to identify the trigger condition and execution of the first action specified by the pathway may further comprise determining that a difference between the value of the data point and the expected value may be greater than the predetermined threshold. In some embodiments, receiving for configuration of the pathway, one of the condition of the user or frequency of triggering the trigger condition upon which to trigger the second level alert and the second action to take subsequent to triggering of the first level alert and the first action further comprises monitoring the data point by receiving data from a sensor measuring the value of the data point. In some embodiments, the first action may include actuating an actuator coupled to the user. In some embodiments, the second action may include actuating the actuator coupled to the user.

In some embodiments, the device may adjust the expected value of the data point based on the first action, responsive to executing the first action. In some embodiments, the device may adjust an expected value of a second data point of the pathway based on the first action, the second data point of the user identified in the specification for the pathway as dependent on the first data point and the first action, responsive to executing the first action. In some embodiments, executing the first action may include selecting, from a plurality of candidate actions, the first action for execution. In some embodiments, selecting, from the plurality of candidate actions, the first action for execution may further comprise determining, from a client device of the user, contextual information pertaining to the user via one or more monitors of the pathway engine. In some embodiments, selecting, from the plurality of candidate actions, the first action for execution may further comprise selecting, using the contextual information, the first action for execution from the plurality of candidate actions. In some embodiments, the contextual information may include location information from the client device of the user. In some embodiments, the contextual information may include a plurality of physiological measurements.

In at least one aspect, the present disclosure is directed to a method for interoperability between pathways configured and executable on a device. A device may execute a plurality of different pathways. Each pathway of the plurality of different pathways may be configured to monitor a defined set of data points of a user and execute actions based on monitoring of the defined set of data points. A first pathway of the plurality of different pathways may, responsive to monitoring values of a first defined set of data points of the user, determine a point in execution of the first pathway to receive one or more inputs from a second pathway of the plurality of different pathways. The first pathway may receive, from the second pathway, as input one or more data values of a second defined set of data being monitored by the second pathway. The first pathway may determine, using at least the values of the first defined set of data points and one or more values of the second defined set of data, to take a predetermined action of the first pathway.

In some embodiments, determining the point in execution of the first pathway to receive the one or more inputs from the second pathway of the plurality of different pathways may further comprise setting the point in execution in accordance with a data transfer schedule. The data transfer schedule may specify a time at which to execute the first pathway to receive the one or more inputs from the second pathway. In some embodiments, receiving as input one or more data values of a second defined set of data being monitored by the second pathway may further comprise selecting the second pathway as the input based on a pathway selection policy. The pathway selection policy may specify which of the plurality of different pathways to select as inputs for the first pathway.

In some embodiments, determining, using at least the values of the first defined set of data points and one or more values of the second defined set of data, to take the predetermined action of the first pathway may further comprise determining a relevancy score of the one or more data values of the second defined set of data to the first pathway. In some embodiments, determining, using at least the values of the first defined set of data points and one or more values of the second defined set of data, to take the predetermined action of the first pathway may further comprise comparing the relevancy score to a relevancy score threshold for the first pathway and the second pathway. In some embodiments, determining, using at least the values of the first defined set of data points and one or more values of the second defined set of data, to take the predetermined action of the first pathway may further comprise taking the predetermined action of the first pathway, responsive to determining that the relevancy score may be greater than the relevancy score threshold.

In some embodiments, determining, using at least the values of the first defined set of data points and one or more values of the second defined set of data, to take the predetermined action of the first pathway may further comprise determining a correlation metric between the values of the first defined set of data points and the one or more values of the second defined set of data. In some embodiments, determining, using at least the values of the first defined set of data points and one or more values of the second defined set of data, to take the predetermined action of the first pathway may further comprise comparing the correlation metric to a correlation threshold for the first pathway and the second pathway. In some embodiments, determining, using at least the values of the first defined set of data points and one or more values of the second defined set of data, to take the predetermined action of the first pathway may further comprise taking the predetermined action of the first pathway responsive to determining that the correlation metric may be greater than the correlation threshold.

In some embodiments, the device may maintain a model for the plurality of different pathways based on the defined set of data points from each of the plurality of different pathways. In some embodiments, receiving, from the second pathway, as input the one or more data values of the second defined set of data being monitored by the second pathway may further comprise calculating the relevancy score for the defined set of data points to the first pathway based on the model. In some embodiments, receiving, from the second pathway, as input the one or more data values of the second defined set of data being monitored by the second pathway may further comprise taking the predetermined action of the first pathway based on the relevancy score to the first pathway.

In some embodiments, the device may maintain a first model for the plurality of different pathways based on the defined set of data points for the user from the plurality of different pathways. In some embodiments, the device may maintain a second model for the plurality of different pathways based on a population defined set of data points for a population of users from the plurality of different pathways. In some embodiments, determining, using at least the values of the first defined set of data points and one or more values of the second defined set of data, to take the predetermined action of the first pathway may further comprise using the first model and the second model to take a predetermined action of the first pathway.

In some embodiments, receiving, from the second pathway, as input one or more data values of a second defined set of data being monitored by the second pathway may further comprise selecting as input the one or more data values of the second defined set of data being monitored by the second pathway based on the first model and the second model. In some embodiments, using at least the values of the first defined set of data points and one or more values of the second defined set of data, to take the predetermined action of the first pathway may further comprise generating a first metric based on the values of the first defined set of data points of the user. In some embodiments, using at least the values of the first defined set of data points and one or more values of the second defined set of data to take the predetermined action of the first pathway may further comprise generating a second metric based on the values of the first defined set of data points of the user and on the one or more values of the second defined set of data. In some embodiments, using at least the values of the first defined set of data points and one or more values of the second defined set of data to take the predetermined action of the first pathway may further comprise taking the predetermined action of the first pathway based on the first metric and the second metric. In some embodiments, using at least the values of the first defined set of data points and one or more values of the second defined set of data to take the predetermined action of the first pathway may further comprise selecting, from a plurality of candidate actions, the predetermined action to take for the first pathway based on the values of the first defined set of data points and one or more values of the second defined set of data.

In at least one aspect, the present disclosure is directed to a system for interoperability between pathways configured and executable on a device. The system may include a device. The device may be configured to execute, via a pathway engine, a plurality of different pathways, each pathway of the plurality of different pathways configured to monitor a defined set of data points of a user and execute actions based on monitoring of the defined set of data points. A first pathway of the plurality of different pathways may be configured to determine, responsive to monitoring values of a first defined set of data points of the user, a point in execution of the first pathway to receive one or more inputs from a second pathway of the plurality of different pathways. The first pathway may be configured to receive, from the second pathway, as input one or more data values of a second defined set of data being monitored by the second pathway. The first pathway may be configured to determine, using at least the values of the first defined set of data points and one or more values of the second defined set of data, to take a predetermined action of the first pathway.

In some embodiments, the first pathway may be further configured to set the point in execution in accordance with a data transfer schedule. The data transfer schedule may specify a time at which to execute the first pathway to receive the one or more inputs from the second pathway. In some embodiments, the first pathway may be further configured to select the second pathway as the input based on a pathway selection policy. The pathway selection policy may specify which of the plurality of different pathways to select as inputs for the first pathway.

In some embodiments, the first pathway may be further configured to determine a relevancy score of the one or more data values of the second defined set of data to the first pathway. In some embodiments, the first pathway may be further configured to compare the relevancy score to a relevancy score threshold for the first pathway and the second pathway. In some embodiments, the first pathway may be further configured to take the predetermined action of the first pathway responsive to determining that the relevancy score may be greater than the relevancy score threshold.

In some embodiments, the first pathway may be further configured to determine a correlation metric between the values of the first defined set of data points and the one or more values of the second defined set of data. In some embodiments, the first pathway may be further configured to compare the correlation metric to a correlation threshold for the first pathway and the second pathway. In some embodiments, the first pathway may be further configured to take the predetermined action of the first pathway, responsive to determining that the correlation metric may be greater than the correlation threshold.

In some embodiments, the device may be further configured to maintain a model for the plurality of different pathways based on the defined set of data points from each of the plurality of different pathways. In some embodiments, the first pathway may be further configured to calculate the relevancy score for the defined set of data points to the first pathway based on the model and take the predetermined action of the first pathway based on the relevancy score to the first pathway.

In some embodiments, the device may be further configured to maintain a first model for the plurality of different pathways based on the defined set of data points for the user from the plurality of different pathways. In some embodiments, the device may be further configured to maintain a second model for the plurality of different pathways based on a population defined set of data points for a population of users from the plurality of different pathways. In some embodiments, the first pathway may be further configured to use the first model and the second model to take a predetermined action of the first pathway. In some embodiments, the first pathway may be further configured to select as input the one or more data values of the second defined set of data being monitored by the second pathway based on the first model and the second model.

In some embodiments, the device may be further configured to generate a first metric based on the values of the first defined set of data points of the user. In some embodiments, the device may be further configured to generate a second metric based on the values of the first defined set of data points of the user and on the one or more values of the second defined set of data. In some embodiments, the device may be further configured to take the predetermined action of the first pathway based on the first metric and the second metric. In some embodiments, the first pathway may be further configured to select, from a plurality of candidate actions, the predetermined action to take for the first pathway based on the values of the first defined set of data points and one or more values of the second defined set of data.

In at least one aspect, the present disclosure is directed to a method for interjecting a communication to a user in a point of execution of a pathway configurable and executable via a pathway engine on a device. A device may, via a pathway engine, execute a pathway configured to monitor a defined set of data points of a user and execute one or more actions based on monitoring the defined set of data points. A monitor of the pathway engine may monitor a plurality of data points of a user received by the device. The pathway may, responsive to monitoring values of the defined set of data points of a user, determine a point in execution in which to communicate to the user. The pathway may select, from a plurality of different communication schemes, a communication scheme for communicating to the user. The pathway may initiate the selected communication scheme.

In some embodiments, executing the pathway may further comprise receiving, by through the device, a specification for the pathway of a data point to be monitored an expected value of the data point, a trigger condition using a comparison of a value of the data point to be monitored with the expected value to a predetermined threshold, and an alert and an action to take as a result of triggering the trigger condition. In some embodiments, determining the point in execution may further comprise determining the point in execution in which to communicate to the user based on the specification for the pathway as the result of triggering the trigger condition. In some embodiments, selecting the communication scheme may further comprise selecting the communication scheme based on the specification for the pathway as the result of triggering the trigger condition. In some embodiments, initiating the selected communication scheme may further comprise initiating the selected communication scheme, a message of the selected communication scheme including the alert and the action to take as the result of the triggering of the triggering condition.

In some embodiments, determining the point in execution may further comprise determining the point in the execution in which to communicate to the user in accordance with a communication schedule, the communication schedule specifying a time at which to initiate the selected communication scheme for communicating to the user. In some embodiments, determining the point in execution may further comprise determining the point in execution in which to communicate to the user responsive to monitoring second values of a second defined set of data points of the user from second pathway different from the pathway. In some embodiments, determining the point in execution may further comprise receiving, from the second pathway, an indicator specifying the point in execution in which communicate to the user, the indicator generated by the second pathway responsive to a specification for the second pathway.

In some embodiments, selecting the communication scheme may further comprise transmitting a request to establish communications between a navigator and the user responsive to determining the point in the execution to communicate to the user. In some embodiments, the plurality of communication schemes may include at least one of the following: a text message to the mobile phone of the user, a push notification to an application on the mobile phone of the user, a telephone call to one or more telephone numbers of the user, a chat session with user, and a video session with the user.

In at least one aspect, the present disclosure is directed to a method for providing a web interface for monitoring a status of pathways of a plurality of users. A first user interface may, via a first display of a device, display the first user interface displaying a plurality of tiles. Each tile of the plurality of tiles may correspond to a user currently being monitored via one or more executing pathways. Each tile may identify the user, a status of alert in a pathway, and one or more last activities. A second user interface, via a second display of the device, may display the second user interface displaying a first queue of encounters with users currently in progress and a second queue of recently completed user encounters. The first user interface may receive a first selection of a tile of the plurality of tiles to initiate a first encounter with a first user corresponding to the selected tile. The second user interface may receive a second selection of a second user in the first queue to open a second encounter in progress with the second user. The second user interface may receive a third selection of a third user in the second queue to view a third encounter completed with the third user.

In some embodiments, each tile of the plurality tiles may comprise at least one of a name, a contact information, an encounter availability status in the pathway, a health history of the user, a health status of the user, and a treatment plan. In some embodiments, the first queue of encounters may further comprise at least one of a first profile picture of the user, a first login time, and a status of progress and wherein the second queue of encounters further comprises at least one of a second profile picture of the user, a second login time, and a status of completion.

In some embodiments, the first encounter with the first user may further include displaying an encounter preview interface. The encounter preview interface may include at least one of a participant list, a communication scheme selector for establishing communications between the user and a navigator, a first message from the user, and a second message from a navigator. In some embodiments, the second encounter with the second user may include displaying an encounter progress interface. The encounter progress interface may include at least one of a communication scheme indicator for communications established between the user and a navigator, an urgency selector, a health status of the user, and an assent selector for a treatment plan. In some embodiments, the third encounter completed with the third user further comprises displaying an encounter completion interface. The encounter completion interface may include at least one of a communication status indicator for communications completed between the user and a navigator, a health status of the user, an assent indicator for a treatment plan for the user, a first message from the user, and a second message from the navigator.

In some embodiments, the device may determine a status of the alert in the pathway based on a defined set of data points from the pathway. In some embodiments, displaying the first user interface may further include modifying the first user interface responsive to determining the status of the alert in the pathway. In some embodiments, displaying the second user interface may further include modifying the second user interface responsive to determining the status of the alert in the pathway.

In some embodiments, the device may determine a status of the alert in the pathway based on a defined set of data points from a second pathway different from the pathway. In some embodiments, displaying the first user interface may further include modifying the first user interface responsive to determining the status of the alert in the pathway from the defined set from the second pathway. In some embodiments, displaying the first user interface may further include modifying the second user interface responsive to determining the status of the alert in the pathway from the defined set from the second pathway. In some embodiments, the device may display an edit menu for the user, the edit menu including at least one of a name, a contact information, a health history of the user, a health status of the user, and a treatment plan.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIGS. 1A-1D are block diagrams depicting embodiments of computing devices useful in connection with the systems and methods described herein;

FIG. 2 is a block diagram depicting a system for providing a pathway configurable and executable via a pathway engine on a device in accordance with an illustrative embodiment;

FIG. 3 is a flow diagram depicting a method of providing a multi-level pathway configurable and executable via a pathway engine on a device in accordance with an illustrative embodiment;

FIG. 4A-4C are flow diagrams depicting a method of providing a pathway for sleep configurable and executable via a pathway engine on a device in accordance with an illustrative embodiment;

FIG. 5 is a flow diagram depicting a method of providing a pathway for heart failure configurable and executable via a pathway engine on a device in accordance with an illustrative embodiment;

FIGS. 6A-6E are flow diagrams depicting a method of providing a pathway for chronic obstructive pulmonary disease (COPD) configurable and executable via a pathway engine on a device in accordance with an illustrative embodiment;

FIGS. 7A and 7B are flow diagrams depicting a method of providing a pathway for COPD exacerbation configurable and executable via a pathway engine on a device in accordance with an illustrative embodiment;

FIGS. 8A-8D are flow diagrams depicting a method of providing a pathway for COPD maintenance configurable and executable via a pathway engine on a device in accordance with an illustrative embodiment;

FIGS. 9A-9G are flow diagrams depicting a method of providing a pathway for activity configurable and executable via a pathway engine on a device in accordance with an illustrative embodiment;

FIGS. 10A-10E are flow diagrams depicting a method of providing a pathway for engagement configurable and executable via a pathway engine on a device in accordance with an illustrative embodiment;

FIGS. 11A-11E are flow diagrams depicting a method of providing a pathway for diabetes management configurable and executable via a pathway engine on a device in accordance with an illustrative embodiment;

FIGS. 12A-12G are flow diagrams depicting a method of providing a pathway for hypertension configurable and executable via a pathway engine on a device in accordance with an illustrative embodiment;

FIGS. 13A and 13B are flow diagrams depicting a method of providing a pathway for patient health questionnaire (PHQ) configurable and executable via a pathway engine on a device in accordance with an illustrative embodiment;

FIG. 14 is a flow diagram depicting a method for providing a pathway configurable and executable via a pathway engine on a device in accordance with an illustrative embodiment;

FIG. 15 is a flow diagram depicting a method for interoperability between pathways configured and executable on a device in accordance with an illustrative embodiment;

FIG. 16 is a flow diagram depicting a method for interjecting a communication to a user in a point of execution of a pathway configurable and executable via a pathway engine on a device in accordance with an illustrative embodiment; and

FIGS. 17A-17J are block diagrams depicting a user interface for monitoring a status of pathways of a plurality of users in accordance with an illustrative embodiment.

### DETAILED DESCRIPTION

For purposes of reading the description of the various embodiments below, the following descriptions of the sections of the specification and their respective contents may be helpful:

Section A describes a network environment and computing environment which may be useful for practicing embodiments described herein.

Section B describes systems and methods for providing a pathway configurable and executable via a pathway engine in accordance with an illustrative embodiment.

Section C describes systems and methods for providing interoperability between pathways in accordance with an illustrative embodiment.

Section D describes systems and methods for interjecting a communication to a user in a point of execution of a pathway.

Section E describes systems and methods for providing a web interface monitoring status of pathways.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit and scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

### A. Computing and Network Environment

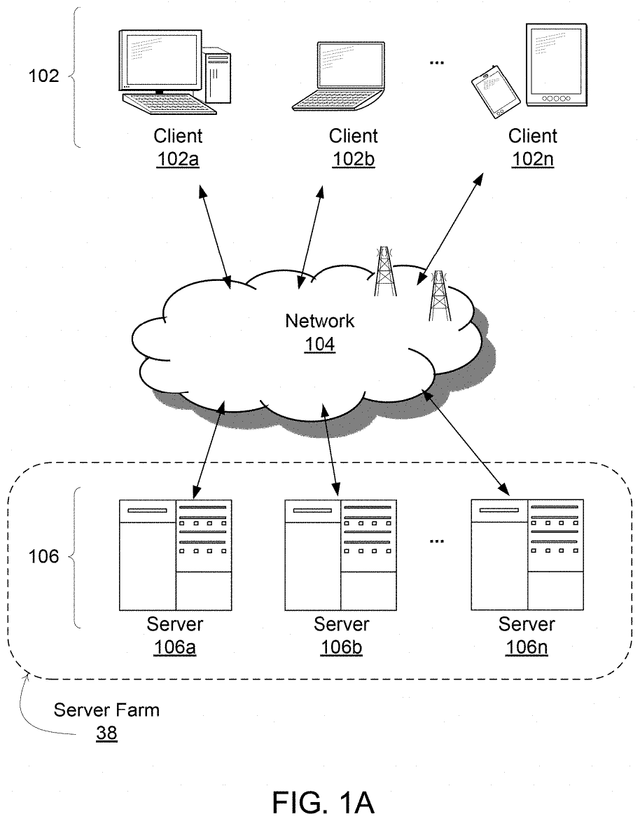

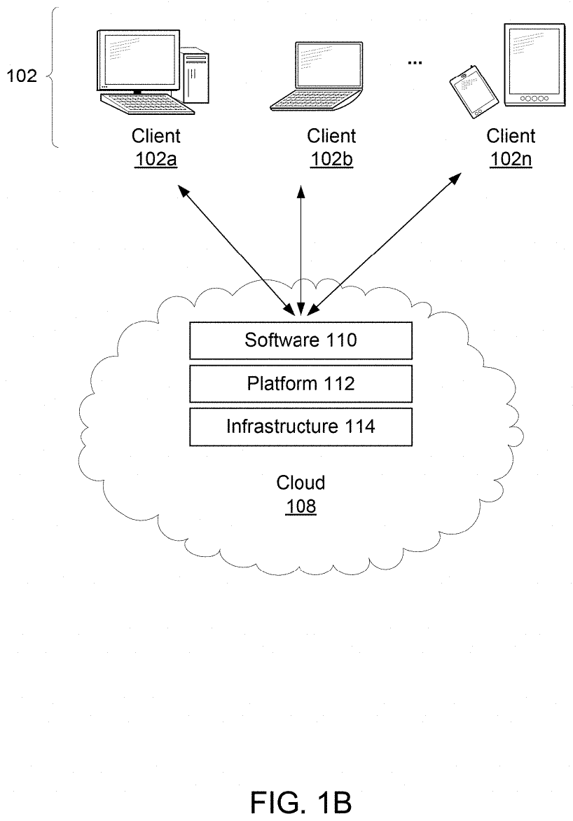

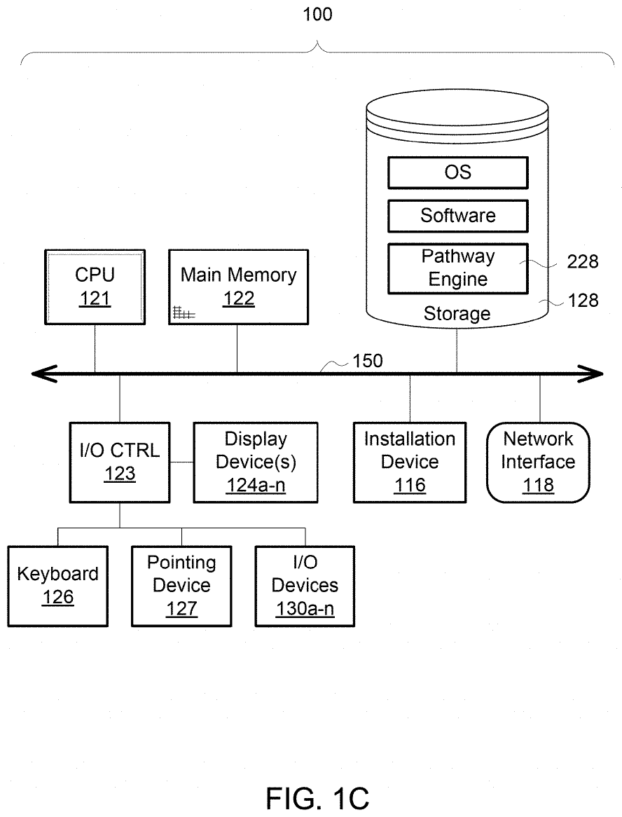

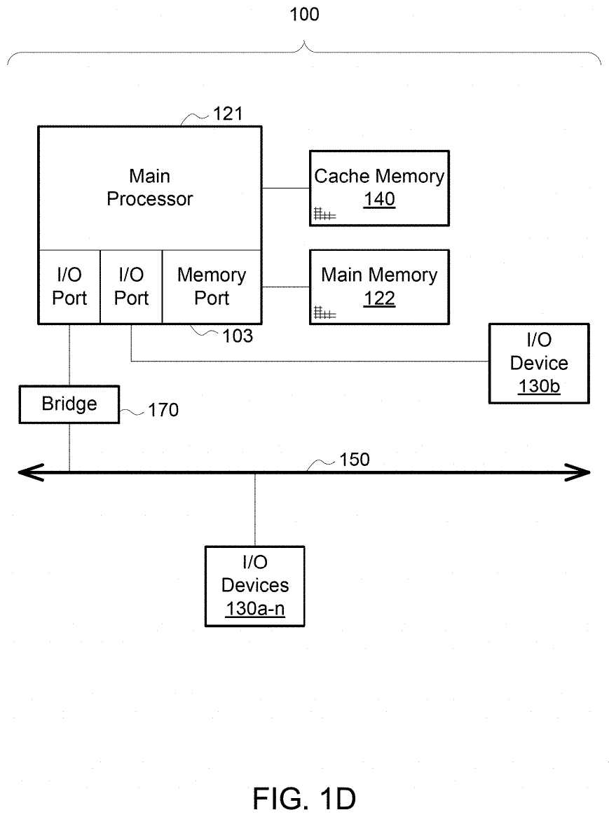

Prior to discussing specific embodiments of the present solution, it may be helpful to describe aspects of the operating environment as well as associated system components (e.g., hardware elements) in connection with the methods and systems described herein. Referring to FIG. 1A, an embodiment of a network environment is depicted. In brief overview, the network environment includes one or more clients 102a-102n (also generally referred to as local machine(s) 102, client(s) 102, client node(s) 102, client machine(s) 102, client computer(s) 102, client device(s) 102, endpoint(s) 102, or endpoint node(s) 102) in communication with one or more servers 106a-106n (also generally referred to as server(s) 106, node(s) 106, or remote machine(s) 106) via one or more networks 104. In some embodiments, a client 102 has the capacity to function as both a client node seeking access to resources provided by a server and as a server providing access to hosted resources for other clients 102a-102n.

Although FIG. 1A shows a network 104 between the clients 102 and the servers 106, the clients 102 and the servers 106 may be on the same network 104. In some embodiments, there are multiple networks 104 between the clients 102 and the servers 106. In some of these embodiments, a network 104′ (not shown) may be a private network and a network 104 may be a public network. In some of these embodiments, a network 104 may be a private network and a network 104′ a public network. In some of these embodiments, networks 104 and 104′ may both be private networks.

The network 104 may be connected via wired or wireless links. Wired links may include a Digital Subscriber Line (DSL), coaxial cable lines, or optical fiber lines. The wireless links may include BLUETOOTH, Wi-Fi, Worldwide Interoperability for Microwave Access (WiMAX), an infrared channel, or satellite band. The wireless links may also include any cellular network standards used to communicate among mobile devices, including standards that qualify as 1G, 2G, 3G, or 4G. The network standards may qualify as one or more generation of mobile telecommunication standards by fulfilling a specification or standards such as the specifications maintained by International Telecommunication Union. The 3G standards, for example, may correspond to the International Mobile Telecommunications-2000 (IMT-2000) specification, and the 4G standards may correspond to the International Mobile Telecommunications-Advanced (IMT-Advanced) specification. Examples of cellular network standards include AMPS, GSM, GPRS, UMTS, LTE, LTE Advanced, Mobile WiMAX, and WiMAX-Advanced. Cellular network standards may use various channel access methods, e.g., FDMA, TDMA, CDMA, or SDMA. In some embodiments, different types of data may be transmitted via different links and standards. In other embodiments, the same types of data may be transmitted via different links and standards.

The network 104 may be any type and/or form of network. The geographical scope of the network 104 may vary widely and the network 104 can be a body area network (BAN), a personal area network (PAN), a local-area network (LAN), e.g., Intranet, a metropolitan area network (MAN), a wide area network (WAN), or the Internet. The topology of the network 104 may be of any form and may include, e.g., any of the following: point-to-point, bus, star, ring, mesh, or tree. The network 104 may be an overlay network which is virtual and sits on top of one or more layers of other networks 104′. The network 104 may be of any such network topology as known to those ordinarily skilled in the art capable of supporting the operations described herein. The network 104 may utilize different techniques and layers or stacks of protocols, including, e.g., the Ethernet protocol, the Internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP Internet protocol suite may include application layer, transport layer, internet layer (including, e.g., IPv6), or the link layer. The network 104 may be a type of a broadcast network, a telecommunications network, a data communication network, or a computer network.

In some embodiments, the system may include multiple, logically-grouped servers 106. In one of these embodiments, the logical group of servers may be referred to as a server farm 38 or a machine farm 38. In another of these embodiments, the servers 106 may be geographically dispersed. In other embodiments, a machine farm 38 may be administered as a single entity. In still other embodiments, the machine farm 38 includes a plurality of machine farms 38. The servers 106 within each machine farm 38 can be heterogeneous—one or more of the servers 106 or machines 106 can operate according to one type of operating system platform (e.g., WINDOWS NT, manufactured by Microsoft Corp. of Redmond, Wash.), while one or more of the other servers 106 can operate on according to another type of operating system platform (e.g., Unix, Linux, or Mac OS X).

In one embodiment, servers 106 in the machine farm 38 may be stored in high-density rack systems, along with associated storage systems, and located in an enterprise data center. In this embodiment, consolidating the servers 106 in this way may improve system manageability, data security, the physical security of the system, and system performance by locating servers 106 and high performance storage systems on localized high performance networks.

Centralizing the servers 106 and storage systems and coupling them with advanced system management tools allows more efficient use of server resources.

The servers 106 of each machine farm 38 do not need to be physically proximate to another server 106 in the same machine farm 38. Thus, the group of servers 106 logically grouped as a machine farm 38 may be interconnected using a wide-area network (WAN) connection or a metropolitan-area network (MAN) connection. For example, a machine farm 38 may include servers 106 physically located in different continents or different regions of a continent, country, state, city, campus, or room. Data transmission speeds between servers 106 in the machine farm 38 can be increased if the servers 106 are connected using a local area network (LAN) connection or some form of direct connection. Additionally, a heterogeneous machine farm 38 may include one or more servers 106 operating according to a type of operating system, while one or more other servers 106 execute one or more types of hypervisors rather than operating systems. In these embodiments, hypervisors may be used to emulate virtual hardware, partition physical hardware, virtualize physical hardware, and execute virtual machines that provide access to computing environments, allowing multiple operating systems to run concurrently on a host computer. Native hypervisors may run directly on the host computer. Hypervisors may include VMware ESX/ESXi, manufactured by VMWare, Inc., of Palo Alto, Calif.; the Xen hypervisor, an open source product whose development is overseen by Citrix Systems, Inc.; the HYPER-V hypervisors provided by Microsoft or others. Hosted hypervisors may run within an operating system on a second software level. Examples of hosted hypervisors may include VMware Workstation and VIRTUALBOX.

Management of the machine farm 38 may be decentralized. For example, one or more servers 106 may comprise components, subsystems and modules to support one or more management services for the machine farm 38. In one of these embodiments, one or more servers 106 provide functionality for management of dynamic data, including techniques for handling failover, data replication, and increasing the robustness of the machine farm 38. Each server 106 may communicate with a persistent store and, in some embodiments, with a dynamic store.

Server 106 may be a file server, application server, web server, proxy server, appliance, network appliance, gateway, gateway server, virtualization server, deployment server, SSL VPN server, or firewall. In one embodiment, the server 106 may be referred to as a remote machine or a node. In another embodiment, a plurality of nodes 290 may be in the path between any two communicating servers.

Referring to FIG. 1B, a cloud computing environment is depicted. A cloud computing environment may provide client 102 with one or more resources provided by a network environment. The cloud computing environment may include one or more clients 102a-102n, in communication with the cloud 108 over one or more networks 104. Clients 102 may include, e.g., thick clients, thin clients, and zero clients. A thick client may provide at least some functionality even when disconnected from the cloud 108 or servers 106. A thin client or a zero client may depend on the connection to the cloud 108 or server 106 to provide functionality. A zero client may depend on the cloud 108 or other networks 104 or servers 106 to retrieve operating system data for the client device. The cloud 108 may include back-end platforms, e.g., servers 106, storage, server farms, or data centers.

The cloud 108 may be public, private, or hybrid. Public clouds may include public servers 106 that are maintained by third parties to the clients 102 or the owners of the clients. The servers 106 may be located off-site in remote geographical locations as disclosed above or otherwise. Public clouds may be connected to the servers 106 over a public network. Private clouds may include private servers 106 that are physically maintained by clients 102 or owners of clients. Private clouds may be connected to the servers 106 over a private network 104. Hybrid clouds 108 may include both the private and public networks 104 and servers 106.

The cloud 108 may also include a cloud-based delivery, e.g., Software as a Service (SaaS) 110, Platform as a Service (PaaS) 112, and Infrastructure as a Service (IaaS) 114. IaaS may refer to a user renting the use of infrastructure resources that are needed during a specified time period. IaaS providers may offer storage, networking, servers, or virtualization resources from large pools, allowing the users to quickly scale up by accessing more resources as needed. Examples of IaaS include AMAZON WEB SERVICES provided by Amazon.com, Inc., of Seattle, Wash., RACKSPACE CLOUD provided by Rackspace U.S., Inc., of San Antonio, Tex., Google Compute Engine provided by Google Inc. of Mountain View, Calif., or RIGHTSCALE provided by RightScale, Inc., of Santa Barbara, Calif. PaaS providers may offer functionality provided by IaaS, including, e.g., storage, networking, servers or virtualization, as well as additional resources such as, e.g., the operating system, middleware, or runtime resources. Examples of PaaS include WINDOWS AZURE provided by Microsoft Corporation of Redmond, Wash., Google App Engine provided by Google Inc., and HEROKU provided by Heroku, Inc. of San Francisco, Calif. SaaS providers may offer the resources that PaaS provides, including storage, networking, servers, virtualization, operating system, middleware, or runtime resources. In some embodiments, SaaS providers may offer additional resources including, e.g., data and application resources. Examples of SaaS include GOOGLE APPS provided by Google Inc., SALESFORCE provided by Salesforce.com Inc. of San Francisco, Calif., or OFFICE 365 provided by Microsoft Corporation. Examples of SaaS may also include data storage providers, e.g., DROPBOX provided by Dropbox, Inc. of San Francisco, Calif., Microsoft SKYDRIVE provided by Microsoft Corporation, Google Drive provided by Google Inc., or Apple ICLOUD provided by Apple Inc. of Cupertino, Calif.

Clients 102 may access IaaS resources with one or more IaaS standards, including, e.g., Amazon Elastic Compute Cloud (EC2), Open Cloud Computing Interface (OCCI), Cloud Infrastructure Management Interface (CIMI), or OpenStack standards. Some IaaS standards may allow clients access to resources over HTTP, and may use Representational State Transfer (REST) protocol or Simple Object Access Protocol (SOAP). Clients 102 may access PaaS resources with different PaaS interfaces. Some PaaS interfaces use HTTP packages, standard Java APIs, JavaMail API, Java Data Objects (JDO), Java Persistence API (JPA), Python APIs, web integration APIs for different programming languages including, e.g., Rack for Ruby, WSGI for Python, or PSGI for Perl, or other APIs that may be built on REST, HTTP, XML, or other protocols. Clients 102 may access SaaS resources through the use of web-based user interfaces, provided by a web browser (e.g., GOOGLE CHROME, Microsoft INTERNET EXPLORER, or Mozilla FIREFOX provided by Mozilla Foundation of Mountain View, Calif.). Clients 102 may also access SaaS resources through smartphone or tablet applications, including, e.g., Salesforce Sales Cloud or Google Drive app. Clients 102 may also access SaaS resources through the client operating system, including, e.g., Windows file system for DROPBOX.

In some embodiments, access to IaaS, PaaS, or SaaS resources may be authenticated. For example, a server or authentication server may authenticate a user via security certificates, HTTPS, or API keys. API keys may include various encryption standards such as, e.g., Advanced Encryption Standard (AES). Data resources may be sent over Transport Layer Security (TLS) or Secure Sockets Layer (SSL).

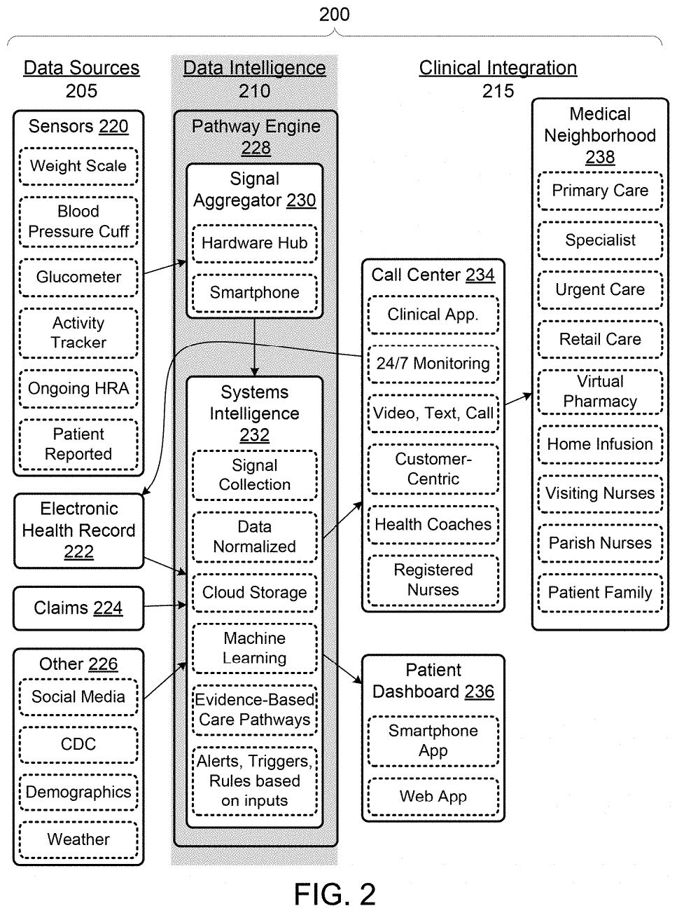

The client 102 and server 106 may be deployed as and/or executed on any type and form of computing device, e.g., a computer, network device or appliance capable of communicating on any type and form of network and performing the operations described herein. FIGS. 1C and 1D depict block diagrams of a computing device 100 useful for practicing an embodiment of the client 102 or a server 106. As shown in FIGS. 1C and 1D, each computing device 100 includes a central processing unit 121, and a main memory unit 122. As shown in FIG. 1C, a computing device 100 may include a storage device 128, an installation device 116, a network interface 118, an I/O controller 123, display devices 124a-124n, a keyboard 126 and a pointing device 127, e.g., a mouse. The storage device 128 may include, without limitation, an operating system, software, and a pathway engine 228 are described below with respect to the description of FIG. 2 below. As shown in FIG. 1D, each computing device 100 may also include additional optional elements, e.g., a memory port 103, a bridge 170, one or more input/output devices 130a-130n (generally referred to using reference numeral 130), and a cache memory 140 in communication with the central processing unit 121.

The central processing unit 121 is any logic circuitry that responds to and processes instructions fetched from the main memory unit 122. In many embodiments, the central processing unit 121 is provided by a microprocessor unit, e.g., those manufactured by Intel Corporation of Mountain View, Calif.; those manufactured by Motorola Corporation of Schaumburg, Ill.; the ARM processor and TEGRA system on a chip (SoC) manufactured by Nvidia of Santa Clara, Calif.; the POWER7 processor, those manufactured by International Business Machines of White Plains, N.Y.; or those manufactured by Advanced Micro Devices of Sunnyvale, Calif. The computing device 100 may be based on any of these processors, or any other processor capable of operating as described herein. The central processing unit 121 may utilize instruction level parallelism, thread level parallelism, different levels of cache, and multi-core processors. A multi-core processor may include two or more processing units on a single computing component. Examples of multi-core processors include the AMD PHENOM IIX2, INTEL CORE i5 and INTEL CORE i7.

Main memory unit 122 may include one or more memory chips capable of storing data and allowing any storage location to be directly accessed by the microprocessor 121. Main memory unit 122 may be volatile and faster than storage 128 memory. Main memory units 122 may be dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 122 or the storage 128 may be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAIVI), Silicon-Oxide-Nitride-Oxide-Silicon (SONOS), Resistive RAM (RRAM), Racetrack, Nano-RAM (NRAM), or Millipede memory. The main memory 122 may be based on any of the above described memory chips, or any other available memory chips capable of operating as described herein. In the embodiment shown in FIG. 1C, the processor 121 communicates with main memory 122 via a system bus 150 (described in more detail below). FIG. 1D depicts an embodiment of a computing device 100 in which the processor communicates directly with main memory 122 via a memory port 103. For example, in FIG. 1D the main memory 122 may be DRDRAM.

FIG. 1D depicts an embodiment in which the main processor 121 communicates directly with cache memory 140 via a secondary bus, sometimes referred to as a backside bus. In other embodiments, the main processor 121 communicates with cache memory 140 using the system bus 150. Cache memory 140 typically has a faster response time than main memory 122 and is typically provided by SRAM, BSRAM, or EDRAM. In the embodiment shown in FIG. 1D, the processor 121 communicates with various I/O devices 130 via a local system bus 150. Various buses may be used to connect the central processing unit 121 to any of the I/O devices 130, including a PCI bus, a PCI-X bus, or a PCI-Express bus, or a NuBus. For embodiments in which the I/O device is a video display 124, the processor 121 may use an Advanced Graphics Port (AGP) to communicate with the display 124 or the I/O controller 123 for the display 124. FIG. 1D depicts an embodiment of a computer 100 in which the main processor 121 communicates directly with I/O device 130b or other processors 121′ via HYPERTRANSPORT, RAPIDIO, or INFINIBAND communications technology. FIG. 1D also depicts an embodiment in which local busses and direct communication are mixed: the processor 121 communicates with I/O device 130a using a local interconnect bus while communicating with I/O device 130b directly.

A wide variety of I/O devices 130a-130n may be present in the computing device 100. Input devices may include keyboards, mice, trackpads, trackballs, capacitive sensors, touchpads, touch mice, multi-touch touchpads and touch mice, microphones, multi-array microphones, drawing tablets, cameras, image sensors (e.g., charge-coupled device (CCD) or complementary metal-oxide semiconductor (CMOS) sensors), cameras, single-lens reflex cameras (SLR), digital SLRs (DSLR), camera arrays, motion sensors (e.g., accelerometers, tilt sensors, or inertial measurement units (IMU)), infrared optical sensors, pressure sensors, magnetometer sensors, angular rate sensors, depth sensors, proximity sensors, ambient light sensors, gyroscopic sensors, or other sensors. Output devices may include video displays, graphical displays, refreshable Braille displays, speakers, headphones, and two- or three-dimensional printers such as, but not limited to, inkjet printers, laser printers, thermal printers, and 3D printers using fused deposition modeling (FDM), stereo-lithography, or laser sintering.

Devices 130a-130n may include a combination of multiple input or output devices, including, e.g., Microsoft KINECT, Nintendo Wiimote for the WII, Nintendo WII U GAMEPAD, or Apple IPHONE. Some devices 130a-130n allow gesture recognition inputs through combining some of the inputs and outputs. Some devices 130a-130n provides for facial recognition which may be utilized as an input for different purposes including authentication and other commands. Some devices 130a-130n provides for voice recognition and inputs, including, e.g., Microsoft KINECT, SIRI for IPHONE by Apple, GOOGLE NOW or Google Voice Search.

Additional devices 130a-130n have both input and output capabilities, including, e.g., haptic feedback devices, touchscreen displays, or multi-touch displays. Touchscreen, multi-touch displays, touchpads, touch mice, or other touch sensing devices may use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, may have larger surfaces, such as on a tabletop or on a wall, and may also interact with other electronic devices. Some I/O devices 130a-130n, display devices 124a-124n or group of devices may be augment reality devices. The I/O devices may be controlled by an I/O controller 123 as shown in FIG. 1C. The I/O controller may control one or more I/O devices, such as, e.g., a keyboard 126 and a pointing device 127, e.g., a mouse or optical pen. Furthermore, an I/O device may also provide storage and/or an installation medium 116 for the computing device 100. In still other embodiments, the computing device 100 may provide USB connections (not shown) to receive handheld USB storage devices. In further embodiments, an I/O device 130 may be a bridge between the system bus 150 and an external communication bus, e.g., a USB bus, a SCSI bus, a FireWire bus, an Ethernet bus, a Gigabit Ethernet bus, a Fibre Channel bus, or a Thunderbolt bus.

In some embodiments, display devices 124a-124n may be connected to I/O controller 123. Display devices may include, e.g., liquid crystal displays (LCD), thin film transistor LCD (TFT-LCD), blue phase LCD, electronic papers (e-ink) displays, flexile displays, light emitting diode (LED) displays, digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays may use, e.g., stereoscopy, polarization filters, active shutters, or autostereoscopy. Display devices 124a-124n may also be a head-mounted display (HMD). In some embodiments, display devices 124a-124n or the corresponding I/O controllers 123 may be controlled through or have hardware support for OPENGL or DIRECTX API or other graphics libraries.

In some embodiments, the computing device 100 may include or connect to multiple display devices 124a-124n, which each may be of the same or different type and/or form. As such, any of the I/O devices 130a-130n and/or the I/O controller 123 may include any type and/or form of suitable hardware, software, or combination of hardware and software to support, enable or provide for the connection and use of multiple display devices 124a-124n by the computing device 100. For example, the computing device 100 may include any type and/or form of video adapter, video card, driver, and/or library to interface, communicate, connect or otherwise use the display devices 124a-124n. In one embodiment, a video adapter may include multiple connectors to interface to multiple display devices 124a-124n. In other embodiments, the computing device 100 may include multiple video adapters, with each video adapter connected to one or more of the display devices 124a-124n. In some embodiments, any portion of the operating system of the computing device 100 may be configured for using multiple displays 124a-124n. In other embodiments, one or more of the display devices 124a-124n may be provided by one or more other computing devices 100a or 100b connected to the computing device 100, via the network 104. In some embodiments software may be designed and constructed to use another computer's display device as a second display device 124a for the computing device 100. For example, in one embodiment, an Apple IPAD may connect to a computing device 100 and use the display of the device 100 as an additional display screen that may be used as an extended desktop. One ordinarily skilled in the art will recognize and appreciate the various ways and embodiments that a computing device 100 may be configured to have multiple display devices 124a-124n.

Referring again to FIG. 1C, the computing device 100 may comprise a storage device 128 (e.g., one or more hard disk drives or redundant arrays of independent disks) for storing an operating system or other related software, and for storing application software programs such as any program related to the software 120 for the content distribution system. Examples of storage device 128 include, e.g., hard disk drive (HDD); optical drive, including CD drive, DVD drive, or BLU-RAY drive; solid-state drive (SSD); USB flash drive; or any other device suitable for storing data. Some storage devices may include multiple volatile and non-volatile memories, including, e.g., solid state hybrid drives that combine hard disks with solid state cache. Some storage device 128 may be non-volatile, mutable, or read-only. Some storage device 128 may be internal and connect to the computing device 100 via a bus 150. Some storage device 128 may be external and connect to the computing device 100 via an I/O device 130 that provides an external bus. Some storage device 128 may connect to the computing device 100 via the network interface 118 over a network 104, including, e.g., the Remote Disk for MACBOOK AIR by Apple. Some client devices 100 may not require a non-volatile storage device 128 and may be thin clients or zero clients 102. Some storage device 128 may also be used as an installation device 116, and may be suitable for installing software and programs. Additionally, the operating system and the software can be run from a bootable medium, for example, a bootable CD, e.g. KNOPPIX, a bootable CD for GNU/Linux that is available as a GNU/Linux distribution from knoppix.net.

Client device 100 may also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon.com, Inc. An application distribution platform may facilitate installation of software on a client device 102. An application distribution platform may include a repository of applications on a server 106 or a cloud 108, which the clients 102a-102n may access over a network 104. An application distribution platform may include application developed and provided by various developers. A user of a client device 102 may select, purchase, and/or download an application via the application distribution platform.

Furthermore, the computing device 100 may include a network interface 118 to interface to the network 104 through a variety of connections including, but not limited to, standard telephone lines, LAN or WAN links (e.g., 802.11, T1, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMAX and direct asynchronous connections). In one embodiment, the computing device 100 communicates with other computing devices 100′ via any type and/or form of gateway or tunneling protocol e.g., Secure Socket Layer (SSL) or Transport Layer Security (TLS), or the Citrix Gateway Protocol manufactured by Citrix Systems, Inc. of Ft. Lauderdale, Fla. The network interface 118 may comprise a built-in network adapter, network interface card, PCMCIA network card, EXPRESSCARD network card, card bus network adapter, wireless network adapter, USB network adapter, modem, or any other device suitable for interfacing the computing device 100 to any type of network capable of communication and performing the operations described herein.

A computing device 100 of the sort depicted in FIGS. 1B and 1C may operate under the control of an operating system, which controls scheduling of tasks and access to system resources. The computing device 100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to, WINDOWS 2000, WINDOWS Server 2012, WINDOWS CE, WINDOWS Phone, WINDOWS XP, WINDOWS VISTA, and WINDOWS 7, WINDOWS RT, and WINDOWS 8 all of which are manufactured by Microsoft Corporation of Redmond, Wash.; MAC OS and iOS, manufactured by Apple, Inc. of Cupertino, Calif.; and Linux, a freely-available operating system, e.g., Linux Mint distribution (“distro”) or Ubuntu, distributed by Canonical Ltd. of London, United Kingdom; or Unix or other Unix-like derivative operating systems; and Android, designed by Google, of Mountain View, Calif., among others. Some operating systems, including, e.g., CHROME OS by Google, may be used on zero clients or thin clients, including, e.g., CHROMEBOOKS.

The computer system 100 can be any workstation, telephone, desktop computer, laptop or notebook computer, netbook, ULTRABOOK, tablet, server, handheld computer, mobile telephone, smartphone or other portable telecommunications device, media playing device, a gaming system, mobile computing device, or any other type and/or form of computing, telecommunications or media device that is capable of communication. The computer system 100 has sufficient processor power and memory capacity to perform the operations described herein. In some embodiments, the computing device 100 may have different processors, operating systems, and input devices consistent with the device. The Samsung GALAXY smartphones, e.g., operate under the control of the Android operating system developed by Google, Inc. GALAXY smartphones receive input via a touch interface.

In some embodiments, the computing device 100 is a gaming system. For example, the computer system 100 may comprise a PLAYSTATION 3, PLAYSTATION 4, PERSONAL PLAYSTATION PORTABLE (PSP), or PLAYSTATION VITA device manufactured by the Sony Corporation of Tokyo, Japan, a NINTENDO DS, NINTENDO 3DS, NINTENDO WII, or NINTENDO WII U device manufactured by Nintendo Co., Ltd., of Kyoto, Japan, or an XBOX 360 or XBOX ONE device manufactured by the Microsoft Corporation of Redmond, Wash.

In some embodiments, the computing device 100 is a digital audio player such as the Apple IPOD, IPOD Touch, and IPOD NANO lines of devices, manufactured by Apple Computer of Cupertino, Calif. Some digital audio players may have other functionality, including, e.g., a gaming system or any functionality made available by an application from a digital application distribution platform. For example, the IPOD Touch may access the Apple App Store. In some embodiments, the computing device 100 is a portable media player or digital audio player supporting file formats including, but not limited to, MP3, WAV, M4A/AAC, WMA Protected AAC, AIFF, Audible audiobook, Apple Lossless audio file formats and .mov, .m4v, and .mp4 MPEG-4 (H.264/MPEG-4 AVC) video file formats.

In some embodiments, the computing device 100 is a tablet e.g., the IPAD line of devices by Apple; GALAXY TAB family of devices by Samsung; or KINDLE FIRE by Amazon.com, Inc. of Seattle, Wash. In other embodiments, the computing device 100 is an e-book reader, e.g., the KINDLE family of devices by Amazon.com, or NOOK family of devices by Barnes & Noble, Inc. of New York City, N.Y.

In some embodiments, the communications device 102 includes a combination of devices, e.g., a smartphone combined with a digital audio player or portable media player. For example, one of these embodiments is a smartphone, e.g. the IPHONE family of smartphones manufactured by Apple, Inc.; a Samsung GALAXY family of smartphones manufactured by Samsung, Inc.; or a Motorola DROID family of smartphones. In yet another embodiment, the communications device 102 is a laptop or desktop computer equipped with a web browser and a microphone and speaker system, e.g., a telephony headset. In these embodiments, the communications devices 102 are web-enabled and can receive and initiate phone calls. In some embodiments, a laptop or desktop computer is also equipped with a webcam or other video capture device that enables video chat and video call.

In some embodiments, the status of one or more machines 102, 106 in the network 104 is monitored, generally as part of network management. In one of these embodiments, the status of a machine may include an identification of load information (e.g., the number of processes on the machine, CPU, and memory utilization), of port information (e.g., the number of available communication ports and the port addresses), or of session status (e.g., the duration and type of processes, and whether a process is active or idle). In another of these embodiments, this information may be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

### B. Systems and Methods for Providing a Pathway Configurable and Executable via a Pathway Engine

To facilitate processing of data from various sources to facilitate medical care, an architecture may include three layers to collect data, to analyze the aggregated data, and to take actions upon processing of the data. In various embodiments, the architecture may include a data intelligence layer. In some embodiments, signals are processed in real time for priority alerts into the mission control dashboard and to the individual user, then processed using unique algorithms that weight various factors including individual history, individual preferences for care and service, current live biometrics, and evidence-based care plans that have been digitized and sorted into a series of rules, alerts, triggers, and algorithms. In some embodiments, data is aggregated and processed in real time in a secure cloud environment.

In various embodiments, the architecture may include a clinical or other integration layer. In some embodiments, the unique utilities of the data intelligence layer provide dashboard or “mission control” views to a live human support group that can use the individualized data as well as population level trending data to provide contextual and individualized decision support, care, and recommendations to end users (including coordinated transfers to outside services). In some embodiments, visualizations of the data are also provided to the end-user.

According to various embodiments, systems and methods for facilitating medical care include conversion of evidence-based knowledge into dynamic individualized care pathways. In some embodiments, a utility takes large bodies of medical (or other) guidelines, research, and subject matter data into a single aggregated location for expert review. In some embodiments, the data is then re-written and re-formulated into new best practice protocols. In some embodiments, these protocols are then loaded into a software system that uses rules, triggers, and a conditional logic layer, a streaming/predictive analytics layer, and ultimately a machine learning layer to process the data for different functions to the end user. In some embodiments, as the utility gathers data on larger and larger populations, the machine learning layer creates new segmentations, discovers new aberrations, reveals undiscovered connected data points, and provides new recommendations for fine tuning responses to groups with “like” data structures. In some embodiments, this process allows for the acceleration of evidence-driven individualized recommendations and decision support for the end-user that will improve expected and desired outcomes.

According to various embodiments, systems and methods for facilitating medical care include a real-time evidence-based mission control dashboard. In some embodiments, a utility presents multiple data points to a customer and to support individuals and allows for highly customized, evidence-driven recommendations and decision support. In some embodiments, the presentation layer provides near real-time sensor data (e.g., wearables, medical sensors, proximity sensors, passive and active sensing), presents near real-time behavioral health sensing data, and integrates views of an individual's previous decisions, preferences, and history from data points including social records, claims records, electronic health records, and individual inputs. In some embodiments, the utility allows for easy browsing, or capability to drill down into individual data points to see source materials. In some embodiments, the utility allows for live text, video, and phone communications that can be recorded, archived, and made searchable for future reference. In some embodiments, the utility also includes functionality allowing for transfer and scheduling of transfers to outside services (a company, a hospital, a doctor, a community resource, another online service, etc.).

According to various embodiments, systems and methods for facilitating medical care include a dynamic behavior change user experience (UX) or user interface (UI) design layer. In some embodiments, a utility supports the creation of dynamic and evidence-driven behavior change suggestions to a device or design layer. In some embodiments, the utility uses customized algorithms based on the user's previous inputs, their current biometrics, their current evidence-based goals and pathways, and their preferences. In some embodiments, using machine learning and human-segmentation and behavior change rules, users are presented with contextual design changes to their interface based on a database of possible segmented designs (e.g., color, tiles, word choice, animations, content presentation, interactive requests, etc.). In some embodiments, these may include alerts, reminders, “nudges,” and/or “kudos” to encourage behavior change and engagement.

According to various embodiments, systems and methods for facilitating medical care include a custom messaging engine for delivering individualized messaging. In some embodiments, a utility uses customer relationship management profiles to gather data from smartphones, smart devices, wearable sensors, and/or other Internet of Things (IoT) connected hardware into a single aggregated data profile. In some embodiments, using custom algorithms and human-curated communication segmentation sets, custom messaging is created to offer support, kudos, encouragement, and customer service messaging that is highly customized to a single individual and takes into account their age, location, interests, device use, sensor signals, and previous inputs into the system. In some embodiments, these customized messages are delivered to web applications, smart devices, or over phone, email, text, or even video chat when delivered by customer service support teams.

According to various embodiments, systems and methods for facilitating medical care include a use of conditional logic and data inputs to make evidence-based guidelines dynamic. In some embodiments, a utility that takes large bodies of medical (or other) guidelines, research, and subject matter data into a single aggregated location for expert review. In some embodiments, the data is then re-written and re-formulated into new best practice protocols. In some embodiments, these protocols are then loaded into a software system that uses rules, triggers, and a conditional logic layer to make the protocol dynamic based on user input, sensor input, or other data inputs. In some embodiments, the protocol will adapt to these inputs to provide a faster, more accurate, and more customized paths from assessment to action.

According to various embodiments, systems and methods for facilitating medical care include automated and digital clinical or care pathways. A clinical pathway is a multidisciplinary management tool based on evidence-based practice for a specific group of patients with a predictable clinical course, in which the different tasks (interventions) by the professionals involved in the patient care are defined, optimized, and sequenced either by hour (ED), day (acute care), or visit (homecare). Outcomes may be tied to specific interventions. In some embodiments, a customer may provide various health-related inputs and the system may identify and assess the inputs based on preprogrammed clinical pathway rules. Inputs may include various health-related measurements of a customer, such as, but not limited to, blood pressure, weight, activity, customer responses to questions, or the like.

In some embodiments, the system may include generalized or core clinical pathways for general health-related issues, such as, but not limited to, blood pressure, sleep, physical activity, or the like. In some embodiments, the system may include specific clinical pathways for particular health-related issues (e.g., specific diseases or conditions), such as, but not limited to, asthma, COPD, or the like. Accordingly, a customer may be subjected to multiple clinical pathways depending on the patient's health condition and history. For example, a patient that has a history of high blood pressure may be placed into the hypertension pathway along with the other more general clinical pathways.

In some embodiments, in response to the measured inputs, the system may compare one or more of the measurements against one or more thresholds, and if a threshold is exceeded by a measurement, to trigger an event. For example, in response to an input of a blood pressure measurement being above a threshold, the system may output a message to the customer or patient indicating that the patient's blood pressure is high. In some embodiments, the response of the system to the inputs may be determined based on how severely the inputs exceed a threshold. For example, if an input severely exceeds a blood pressure threshold, the system may respond more urgently and automatically alert a clinician of the patient's severely high blood pressure condition. The system may then communicatively connect the patient and the clinician such that the clinician can determine the best course of action or provide pertinent medical information to the customer with the high blood pressure. In some embodiments, the system may automatically respond to a health trigger of a customer (e.g., provide a warning message or provide questions to be answered by the customer). In addition, in response to another event, the system may shift the response from automated to live by connecting the customer to a live clinician. For example, if the automated response provides a customer with questions to answer, the system may direct the customer to the live clinician in response to answers to those questions (e.g., if the answers indicate a more severe medical scenario).

In some embodiments, the system may refine and update pathways based on the inputs of the plurality of customers, the actions in response to inputs, the outcomes of the actions, and so on. For example, the system may analyze incoming data and outputs and trending data to create new rules, triggers, and alerts of clinical pathways. For example, if the system determines that a high blood pressure may be a precursor to a more serious health event (e.g., a heart attack), the system may refine a blood pressure pathway to lower the blood pressure threshold at which a customer may receive a warning to be able to prevent the serious health event from occurring.

In some embodiments, the system provides functional and interactive features to customers (e.g., in a mobile app at a smartphone). The system may provide a user experience and user interface, a design, a business process of data movement to allow for upstream and proactive health care, or the like. In some embodiments, the customers may utilize the system via a user interface to provide health-related inputs (e.g., via sensors) to the system for analysis by the system, and the system may provide continuous health risk assessments based on the data inputs (e.g., from the sensors).

Referring now to FIG. 2, FIG. 2 is a block diagram depicting an environment 200 for providing a pathway configurable and executable via a pathway engine 226, in accordance to an illustrative embodiment. In overview, the environment 200 can include a data sources layer 205, a data intelligence layer 210, and a clinical integration layer 215. The data sources layer 205 can include sensors 220, an electronic health record database 224, claims database 224, and other databases 226. The data intelligence layer 210 can include a signal aggregator 230 and systems intelligence 232. The clinical integration layer 215 can include a call center 234, a patient dashboard 236, and a medical neighborhood database 238. Each of the data sources layer 205, a data intelligence layer 210, and a clinical integration layer 215 can be communicatively coupled to one another, for example as shown in FIG. 2. Each of the components in environment 200 can be implemented using the system 100 in FIGS. 1A-1D. The environment 200 can provide individualized interventions, coaching, and recommendations from live connected human support (text, phone, video chat, electronic communication) or automated response through smart device and wearable communication systems.

Live support may be informed by a system dashboard or “mission control” view that provides highly customized and individualized recommendations and decision support tools created through the utilization of custom written algorithms, evidence based data sets, population segmentation rules, and machine learning. This utility may allow the live support system to resolve issues proactively and predictively with the end-user. The utility can also triage or transfer the end-user to an established alternative service using “warm hand offs” or establish connections to alternative services to resolve the end-user's needs using high levels of customer support afforded by the environment 200.

The data sources layer 205 can include health pathways and non-health related pathways. The data sources layer 205 may allow for the aggregation of sensor signals (wearables, proximity, home, patches, ingestible, smart device sensors such as GPS, accelerometer, microphone, etc.), electronic health records, claims data, and other data sources (weather, centers for disease control (CDC) data, social data, etc.) into a signal processing and sorting analytics algorithm (streaming analytics) through an individual smart device or hardware hub. Within the data sources layer 205, the sensors 220 can include one or more devices connected to a patient to measure physiological status of the patient or user. The sensors 220 can include, for example, a weight scale, a blood pressure cuff, a glucometer, an activity tracker, an ongoing health reimbursement arrangement manager, and a patient reported detector.

The data sources layer 205 can include an electronic health record database 222. The electronic health record database 222 can include past health information about the patient. The data sources layer 205 can include a claims database 224. The claims database 224 can include health claims reported by the patient. Other databases 226 can include a social media database, a centers for disease control (CDC) database, demographics database, and weather report database.

Via the data intelligence layer 210, signals may be processed in real time for priority alerts into the mission control dashboard and to the individual user, then may be processed using unique algorithms that weight various factors including individual history, individual preferences for care and service, current live biometrics, and evidence based care plans that have been digitized and sorted into a series of rules, alerts, triggers, and algorithms. Data may be then aggregated and processed in real time in a secure cloud environment. The modules of the data intelligence layer 210 can take large bodies of medical (or other) guidelines, research, and subject matter data into a single aggregated location for expert review at the clinical integration layer 215. The data is then re-written and re-formulated into new best practice protocols. These protocols may be then loaded into a software system that uses rules, triggers, and a conditional logic layer, a streaming/predictive analytics layer, and ultimately a machine learning layer to process the data for different functions to the user. As the data intelligence layer 210 gathers data on larger and larger populations, the machine learning module may create new segmentations, discovers new aberrations, reveals undiscovered connected data points, and provides new recommendations for fine tuning responses to groups with “like” data structures. This process may allow for the acceleration of evidence-driven individualized recommendations and decision support for the end-user that will improve expected and desired outcomes.

The data intelligence layer 210 can include a pathway engine 228. The pathway engine 228 can include a signal aggregator 230 and systems intelligence 232. The signal aggregator 230 can receive and aggregate data from the sensors 220 of the data sources layer 205. The signal aggregator 230 can include a hardware hub and a smartphone. The systems intelligence 232 can include a signal collector, a data normalizer, a cloud storage, a machine learning module, evidence-based care pathways module, and an alerts, triggers, and rules database. The signal aggregator 230 and the systems intelligence 232 can be communicatively coupled to each other. Details of the pathway engine 228 are detailed herein in more detail below.

Moving to the clinical integration layer 215, the clinical integration layer 215 can present multiple data points to a customer and to support individuals and allows for highly customized, evidence-driven recommendations and decision support. The clinical integration layer 215 can provide near real-time sensor data (wearables, medical sensors, proximity sensors, passive and active sensing), presents near real time behavioral health sensing data, and integrates views of an individual's previous decisions, preferences, and history from data points, including social records, claims records, electronic health records, and individual inputs. The clinical integration layer 215 can provide for easy browsing, or capability to drill down into individual data points to see source materials. In addition, the clinical integration layer 215 can provide for live text, video, and phone communications that can be recorded, archived, and made searchable for future reference. The clinical integration layer 215 can provide also include functionality allowing for transfer and scheduling of transfers to outside services (a company, a hospital, a doctor, a community resource, another online service, etc.).

The clinical integration layer 215 can include a call center 234. The call center 234 can include a clinical application, 24/7 monitoring module, video-text-call communications module, customer centric module, health coaches module, and a registered nurse database. The clinical integration layer 215 can include a patient dashboard 236. The patient dashboard 236 can include a smart phone application and/or a web application. The clinical integration layer 215 can also include a medical neighborhood 238. The medical neighborhood 238 can include at least one of a primary care, a specialist, an urgent care, a retail care, a virtual pharmacy, a home infusion, visiting nurses, parish nurses, and a patient family for the patient or user.

The clinical integration layer 215 can support the creation of dynamic and evidence-driven behavior change suggestions to a device or design layer. The clinical integration layer 215 can use customized algorithms based on the user's previous inputs, their current biometrics, their current evidence based goals and pathways, and their preferences. Using machine learning and human segmentation and behavior change rules, users may be presented with contextual design changes to their interface based on a database of possible segmented designs (color, tiles, word choice, animations, content presentation, interactive requests, etc.). These may include alerts and reminder to encourage behavior change and engagement.

The clinical integration layer 215 can create and/or maintain customer relationship management profiles to gather data from smartphones, smart devices, wearable sensors, and other IoT-connected hardware into a single aggregated data profiles. Using custom algorithms and human curated communication segmentation sets, custom messaging may be created to offer support, encouragement, and customer service messaging that is highly customized to a single individual and takes into account their age, location, interests, device use, sensor signals, and previous inputs into the system. These customized messages may be delivered to web applications, smart devices, or over phone, email, text, or even video chat when delivered by customer service support teams.

The clinical integration layer 215 can take large bodies of medical (or other) guidelines, research, and subject matter data into a single aggregated location for expert review. The data may be then re-written and re-formulated into new best practice protocols. These protocols may be then loaded into a software system that uses rules, triggers, and a conditional logic layer to make the protocol dynamic based on user input, sensor input, or other data inputs. The protocol may adapt to these inputs to provide a faster, more accurate, and more customized path from assessment to action.

Focusing on the data intelligence layer 210, a device (e.g., computer system 100) may establish a pathway configured via a pathway engine 228 to execute on the device to monitor one or more data points of the user and to generate one or more actions based on monitoring of the one or more data points. The path way may correspond to one of the components in the data sources layer 205, such as the weight scale, blood pressure cuff, glucometer, activity tracker, ongoing HRA, and the patient reported of the sensors 220, the electronic health record database 222, claims database 224, and the other databases 226 at the data sources layer 205. The pathway engine 228 can manage one or more data points received from the pathway of the data sources layer 205. The pathway engine 228 can generate the one or more actions based on the monitoring of the one or more data points from the data sources layer 205. The one or more actions can include actuating an actuator coupled to the user. The actuator may be part of a device connected to the user, such as that of a medical device at the clinical integration layer 215. In some embodiments, the pathway engine 228 can apply any number of machine learning techniques to determine the one or more actions to take on the patient.

The device may establish a pathway configured via the pathway engine 228 to execute on the device to monitor one or more data points of a user and trigger cascading levels of actions based on monitoring of the one or more data points. The established pathway may correspond to one of the components in the data sources layer 205, such as the weight scale, blood pressure cuff, glucometer, activity tracker, ongoing HRA, and the patient reported of the sensors 220, the electronic health record database 222, claims database 224, and the other databases 226 at the data sources layer 205. The pathway engine 228 of the device may establish bi-directional or unidirectional communications with the pathway configured via the pathway engine 228 with the data sources layer 205. The pathways may use external data, such as weather, environment, social medial, socioeconomic, demographic information regarding the user. The pathways may use data collected by the mobile phone of a patient and devices connected to the mobiles (e.g., Bluetooth-enabled weight-scales, asthma devices, and prescription devices). The pathways may be configurable, adjustable, and dynamic to use different criteria, context, logic, decision points, and interaction points, among others.

The device may receive a specification for the pathway of a data point to be monitored. The specification for the pathway of the data point to be monitored may include configuration information to process the data point from the pathway. The device may receive an expected value of the data point. The expected value of the data point may correspond to measurements of one of the pathways at the data sources layer 205. The device may receive a trigger condition using a comparison of a value of the data point to be monitored with the expected value to a predetermined threshold. The device may receive a trigger condition using a comparison of a difference between a value of the data point to be monitored and the expected value, to the predetermined threshold. The trigger condition may be used by the pathway engine 228 to execute the designated alert or the action. The device may receive an alert and an action to take as a result of triggering the trigger condition.

A monitor of the pathway engine 228 can monitor a plurality of data points of the user received by the device. The monitor of the pathway engine 228 can receive the plurality of data points from one or more components at the data sources layer 205. The monitor of the pathway engine 228 can monitor the data point by receiving data from a sensor measuring the value of the data point. The monitor can receive, for example, weight of the user from the weight scale of the sensors 220, a blood pressure from the blood pressure cuff of the sensors 220, a glucose reading from the glucometer of the sensors 220, an activity reading from the activity tracker of the sensors 220, among others. The signal aggregator 230 of the pathway engine 228 can aggregate the received plurality of data points. The pathway engine 228 can store the aggregated plurality of data points by which sensor 220 the respective data points originated. The pathway engine 228 can process the received plurality of data points using the various modules of the systems intelligence 232, for example, using machine learning, evidence-based care pathways, and alerts, triggers, and rules based on inputs. Machine learning techniques of the systems intelligence module 232 may include artificial neural networks (ANN), nearest neighborhood algorithms (k-NN), reinforcement learning algorithms, Q-learning, and support vector machines (SVMs), among others.

The monitor can compare each value of the plurality of data points to the expected value and the predetermined threshold specified for the trigger condition. The plurality of data points may correspond to data from the one or more components of the data sources layer 205. The expected value may correspond to a simulated or predicted measurement regarding the user from the respective component at the data sources layer 205. In some embodiments, the received expected value may be adjusted by the pathway engine 228. The predetermined threshold may represent a degree of deviation from the expected value to satisfy the trigger condition. In some embodiments, the monitor of the pathway engine 228 can calculate a difference between the value of the data point and the expected value. In some embodiments, the monitor can determine whether the difference between the value of the data point and the expected value is greater than the threshold.

The pathway, based on the comparison, can determine that the trigger condition has been triggered. In some embodiments, there may be multiple trigger conditions. In such embodiments, the pathway can identify which trigger condition has been triggered based on the comparison. If the difference between the value of the data point and the expected value is greater than the threshold, the pathway can determine that the trigger condition has been triggered. If the difference between the value of the data point and the expected value is less than or equal to the threshold, the pathway can continue monitoring the value of the plurality of data points and comparing each value of the plurality of data points to the expected value and the predetermined threshold specified for the trigger condition.

The pathway, responsive to the triggering of the trigger condition, can initiate an alert to identify the trigger condition and execution of the action specified by the pathway. A type of alert may be specific to the trigger condition and the action. The alert may include a message indicating what action the user should take. A type of action may be specific to the trigger condition and the alert. In some embodiments, responsive to identifying the trigger condition, the pathway can cause the device to instantiate a dialogue window containing the alert. In some embodiments, the pathway can identify the alert corresponding to the trigger condition. In some embodiments, the pathway can determine the alert to present to the user based on the modules of the system intelligence 232. For example, the pathway can use machine learning techniques to identify the alert to present. In some embodiments, the pathway can relay the alert to the one or more components of the clinical integration layer 215. In some embodiments, the pathway can identify the action to be executed as specified by the pathway. In some embodiments, the pathway can identify the action to be executed based on the modules of the systems intelligence 232. The alert may be used by the device to communicate to the user, upon triggering of the trigger condition. The action may be used by the device to command another client device to actuate in accordance to the command.

In some embodiments, the device may select the action for execution, from a plurality of candidate actions. Which of the plurality of candidate actions is to be selected may be specified by the pathway. The plurality of candidate actions may include actions to be taken by a client device. In some embodiments, the device can determine, from a client device of the user, contextual information pertaining to the user via the one or more monitors of the pathway engine. The device can select, using the contextual information, the action for execution from the plurality of candidate actions. The device can relay the selected action to the clinical integration layer 215.

In some embodiments, the alert may be differentiated by levels. The pathway can initiate, responsive to the triggering of the trigger condition, the first level alert to identify the trigger condition and execution of the first action specified by the pathway. The first level alert may correspond to an initial alert to the user. For example, the first level alert may correspond to an initial onset of a certain health symptoms that the user may have. First action may correspond to an initial action to be taken by the user. For example, the first level action may correspond to a preliminary action to help offset such health symptoms that the user may have. In addition, the monitor can monitor, responsive to triggering of the trigger condition, values of the plurality of data points for one of the condition of the user or frequency of triggering the trigger condition upon which to trigger the second level alert and the second action.

Responsive to executing the action, the device can adjust the expected value of the data point based on the action. In some embodiments, the device may adjust an expected value of a second data point of the pathway based on the action, the second data point of the user identified in the specification for the pathway as dependent on the first data point and the action, responsive to executing the action. In some embodiments, the pathway can calculate an adjustment to the expected value based on the action using the modules of the system intelligence 232. For example, the pathway can estimate the adjustment to the expected value using machine learning techniques and the action taken. If the user is asked to exercise in response to an alert from a chronic health failure pathway, the pathway can estimate that the likelihood of the user of suffering from such health failure to decrease by a preset percentage.

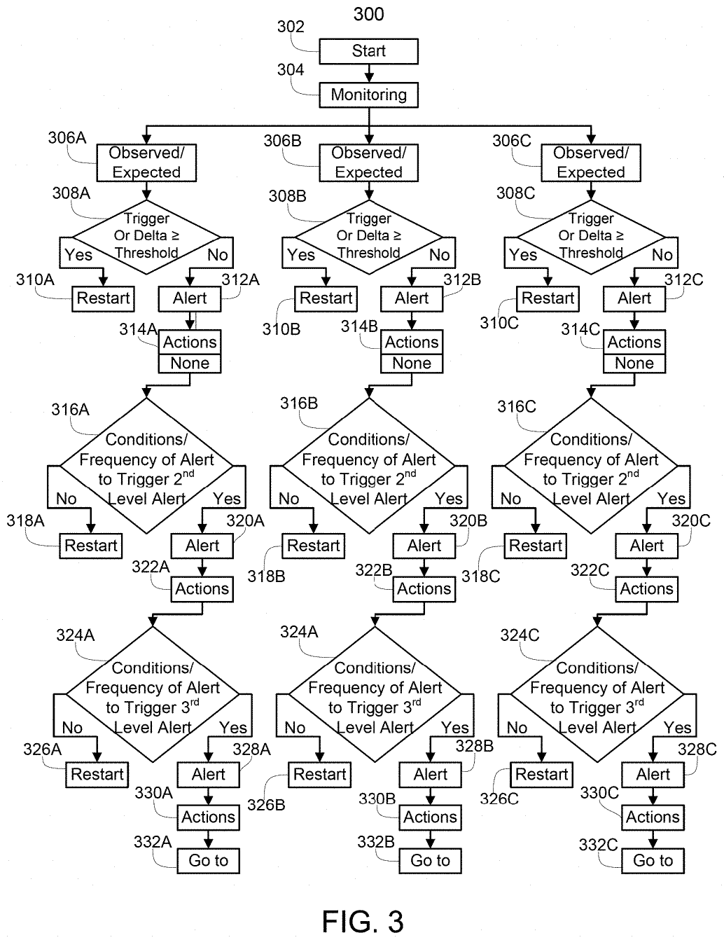

Referring to FIG. 3, FIG. 3 is a flow diagram depicting a method 300 of providing multi-level pathway configurable and executable via a pathway engine on a device, in accordance with an illustrative embodiment. The functionalities of method 300 may be executed by systems 100 and/or 200 as detailed herein in conjunction with FIGS. 1 and 2. In overview, the device may start the process (BLOCK 302). The device may then monitor for one or more data points of a user from a pathway (BLOCK 304). The device can compare the observed one or more data points with expected data points (BLOCKS 306A-306C). The method 300 may branch at BLOCKS 306A-306C. BLOCKS 306A-306C may correspond to three different pathways or data sources. The device can the determine whether the trigger has occurred or delta is greater than or equal to a threshold (BLOCKS 308A-308C). The trigger may be a condition particular to the pathway. The threshold may also be particular to the pathway. If the trigger has not yet occurred or the delta is below a threshold, the device may restart the process (BLOCKS 310A-310C). On the other hand, if the trigger has occurred or the delta is below the threshold, the device may signal an alert to the user (BLOCKS 312A-312C). The device may then perform one of an action or no action (BLOCKS 314A-314C). The action may be specified by the specification for the particular pathway.

The device may then determine whether the condition or frequency of alert to trigger a second alert exist (BLOCKS 316A-316C). The condition or frequency of alert to trigger second alert may correspond to the different pathways or data sources. If the condition or frequency of alert to trigger second alert exist, the device may restart the process (BLOCKS 318A-318C). On the other hand, if the condition or frequency of alert to trigger second alert does not exist, the device may signal an alert to the user (BLOCKS 320A-320C). The device may then perform one of an action or (BLOCKS 322A-322C). The action may be specified by the specification for the particular pathway.

The device may then determine whether the condition or frequency of alert to trigger a third alert exist (BLOCKS 324A-324C). The condition or frequency of alert to trigger second alert may correspond to the different pathways or data sources. If the condition or frequency of alert to trigger second alert exist, the device may restart the process (BLOCKS 326A-326C). On the other hand, if the condition or frequency of alert to trigger second alert does not exist, the device may signal an alert to the user (BLOCKS 328A-328C). The device may then perform one of an action (BLOCKS 330A-330C). The action may be specified by the specification for the particular pathway. The device may then perform a go to (BLOCKS 332A-332C).

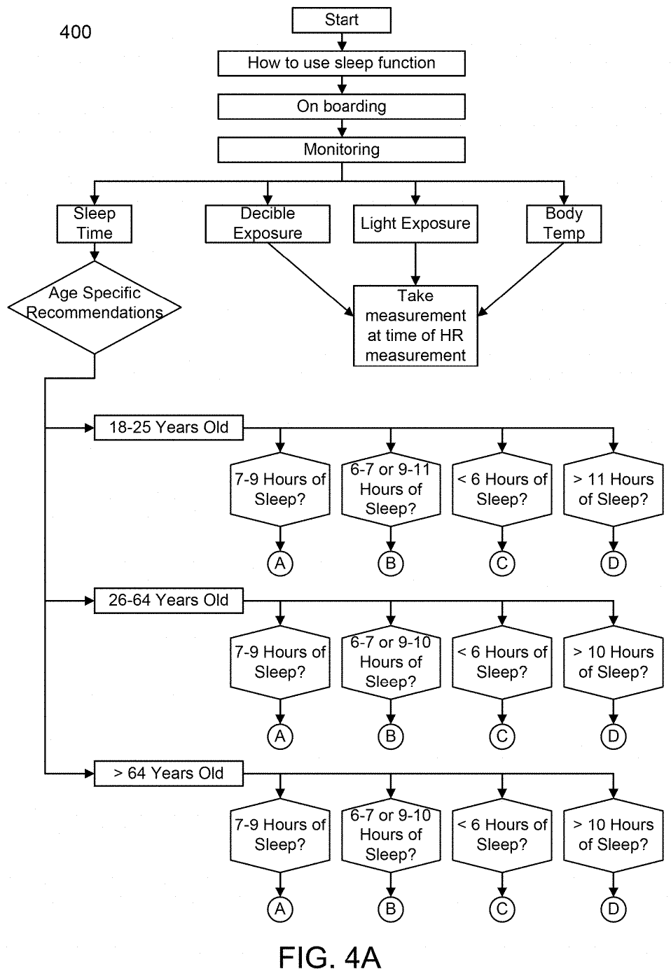

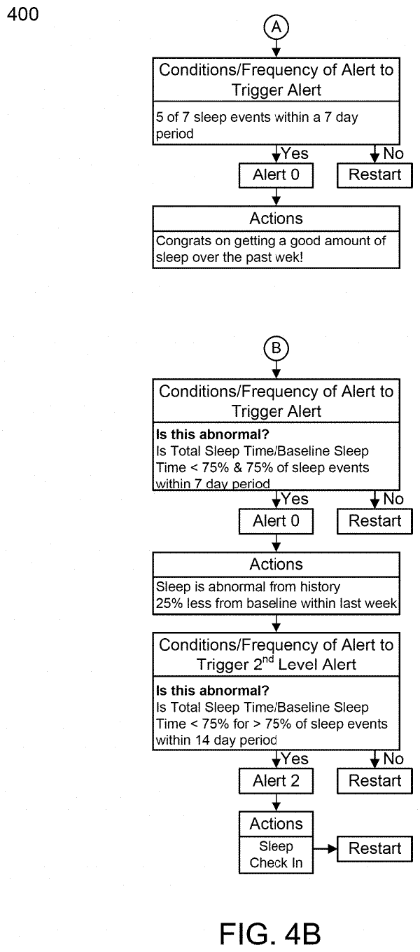

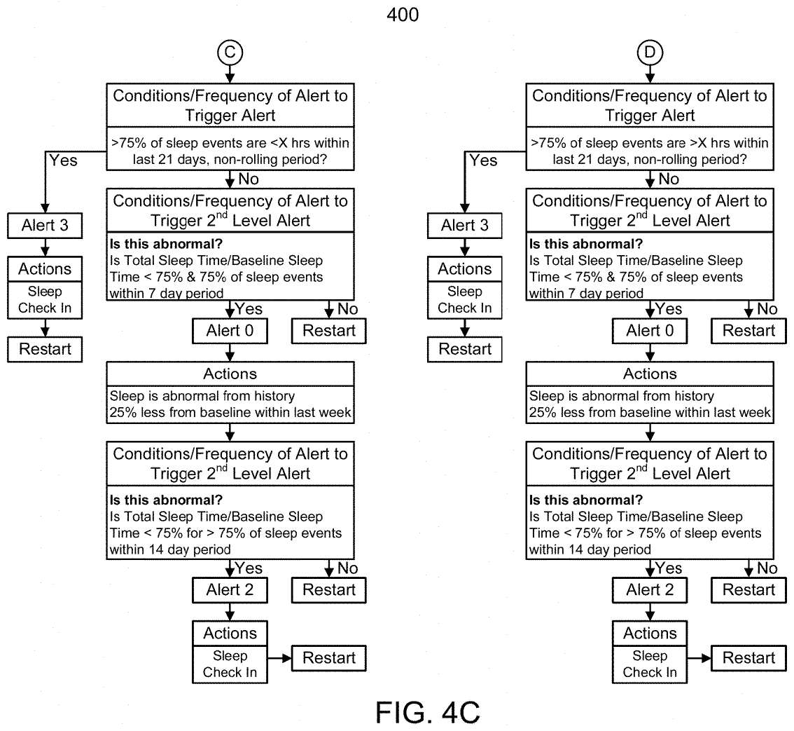

Referring to FIGS. 4A-4C, FIGS. 4A-4C are flow diagrams depicting a method 400 of providing a pathway for sleep configurable and executable via a pathway engine on a device in accordance with an illustrative embodiment. The functionalities of method 400 may be executed by systems 100 and/or 200 as detailed herein in conjunction with FIGS. 1 and 2. The method 400 may be executed by a pathway for sleep to alert a user as regarding the sleep habits of the user.

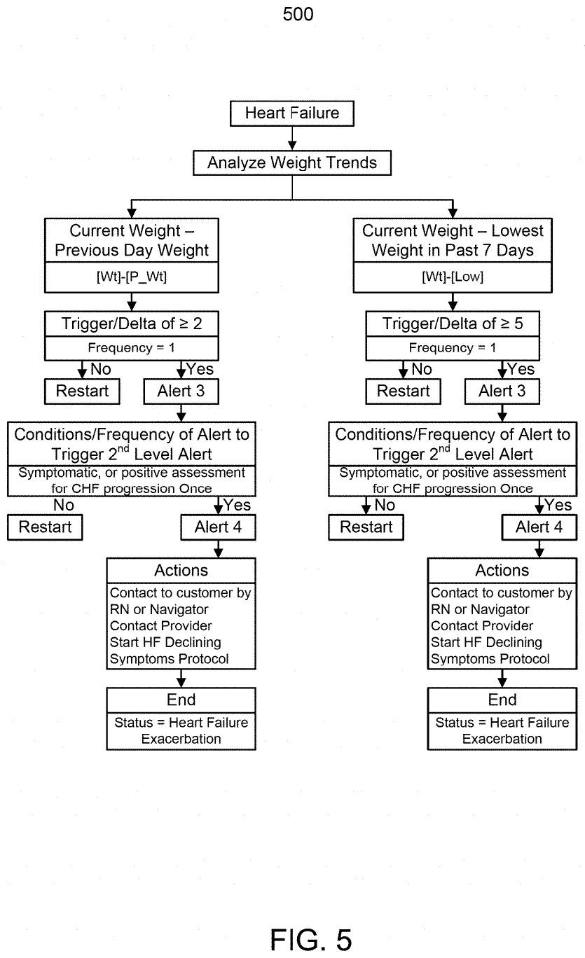

Referring to FIG. 5, FIG. 5 is a flow diagram depicting a method 500 of providing a pathway for heart failure configurable and executable via a pathway engine on a device in accordance with an illustrative embodiment. The functionalities of method 500 may be executed by systems 100 and/or 200 as detailed herein in conjunction with FIGS. 1 and 2. The method 500 may be executed by a pathway for health failure to alert the user regarding chronic heart failure.

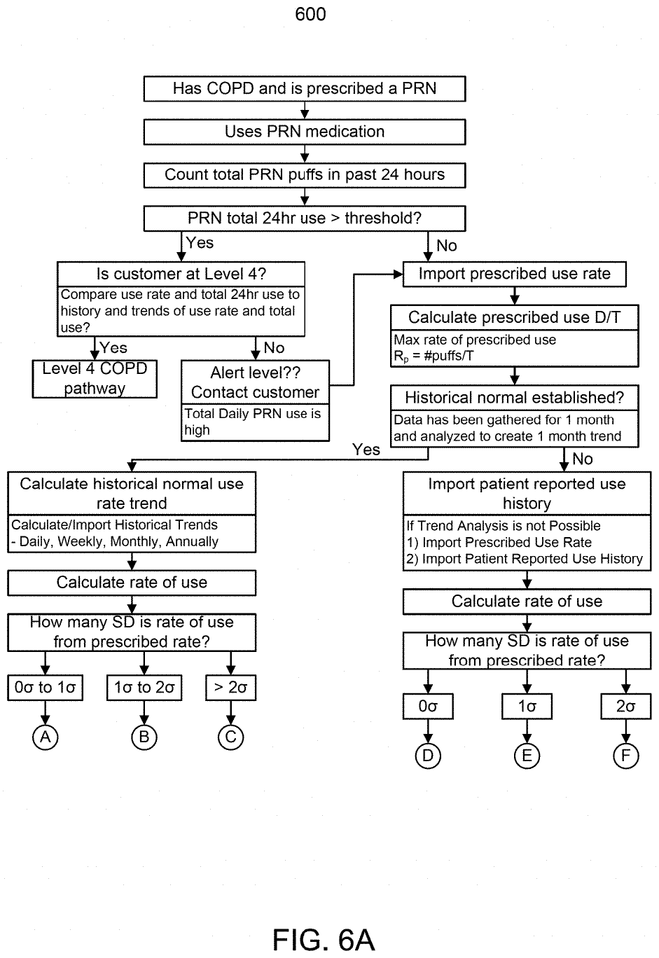

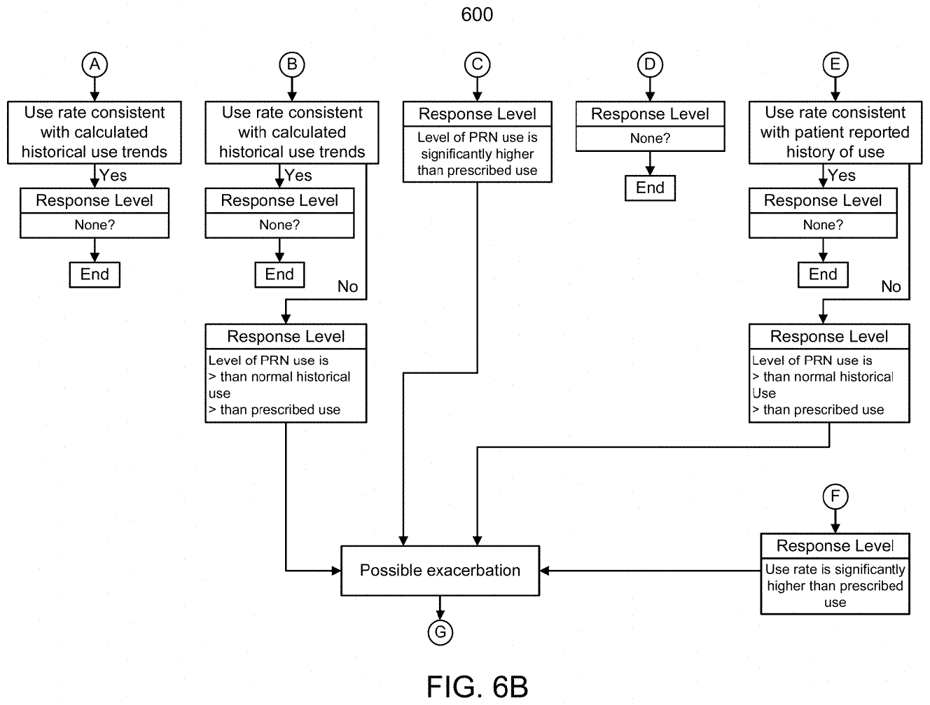

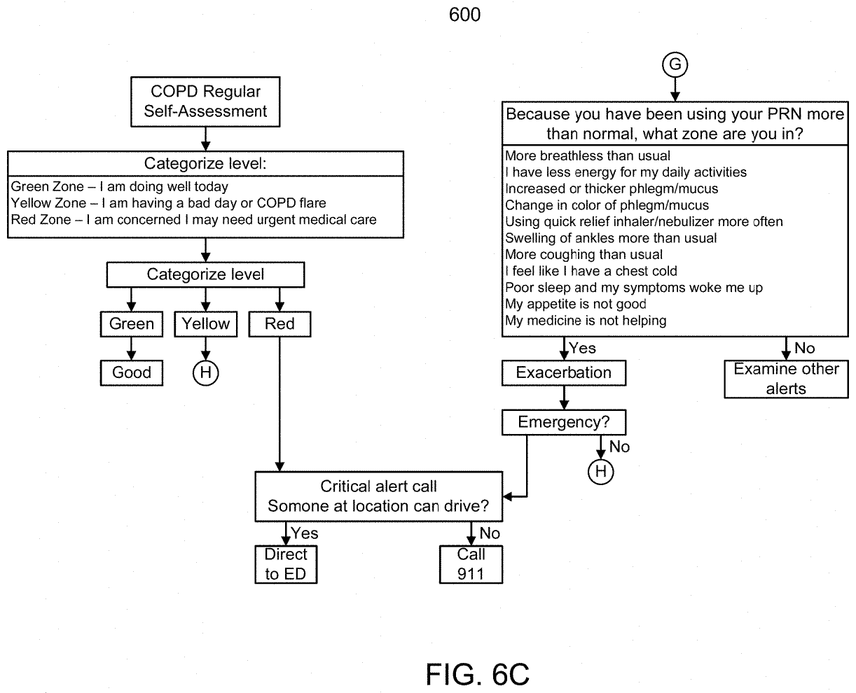

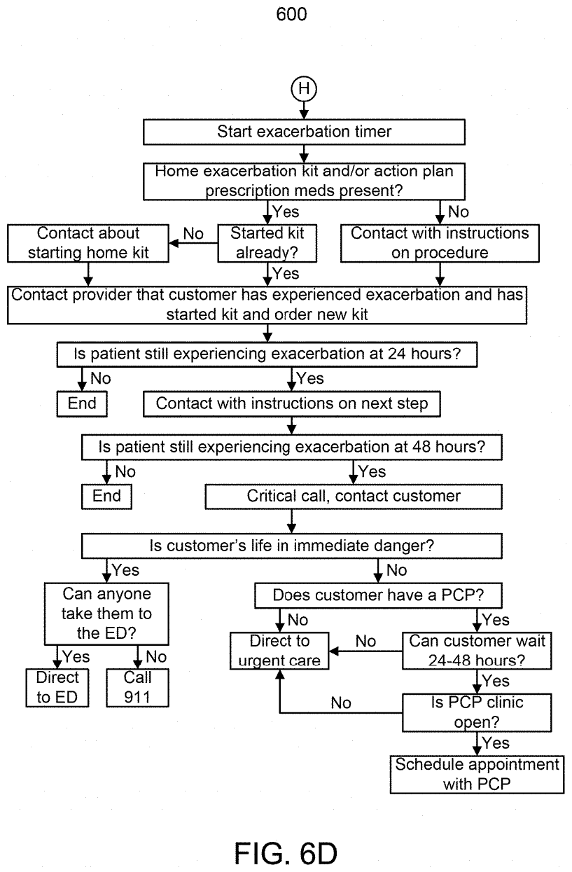

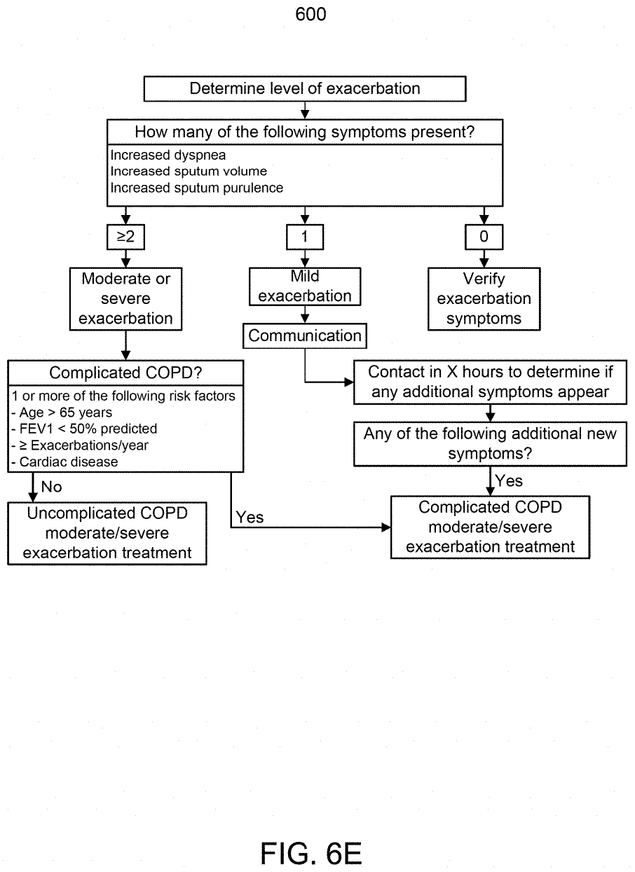

Referring to FIGS. 6A-6E, FIGS. 6A-6E are flow diagrams depicting a method 600 of providing a pathway for chronic obstructive pulmonary disease (COPD) configurable and executable via a pathway engine on a device in accordance with an illustrative embodiment. The functionalities of method 600 may be executed by systems 100 and/or 200 as detailed herein in conjunction with FIGS. 1 and 2. The method 600 may be executed by a pathway for detecting and alerting chronic obstructive pulmonary disease (COPD) for the user.

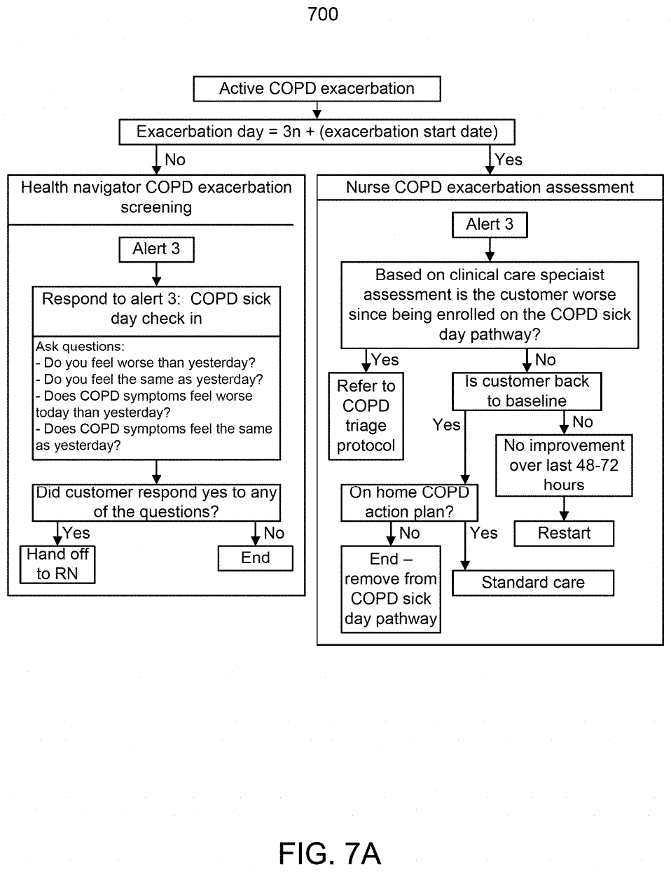

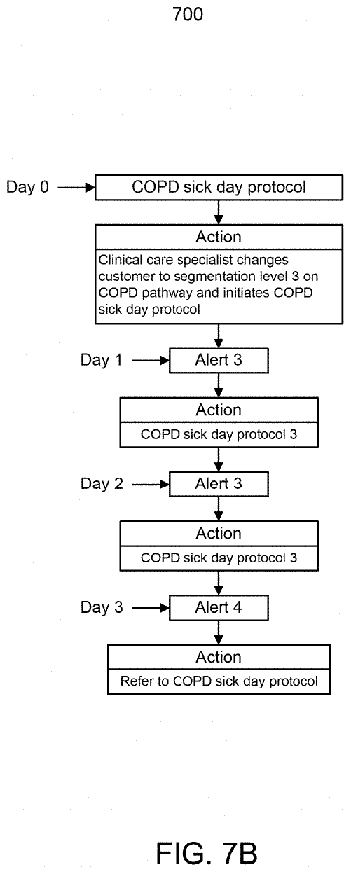

Referring to FIGS. 7A and 7B, FIGS. 7A and 7B are flow diagrams depicting a method 700 of providing a pathway for COPD exacerbation configurable and executable via a pathway engine on a device in accordance with an illustrative embodiment. The functionalities of method 700 may be executed by systems 100 and/or 200 as detailed herein in conjunction with FIGS. 1 and 2. The method 700 may be executed by a pathway for detecting and alerting chronic obstructive pulmonary disease (COPD) exacerbation for the user.

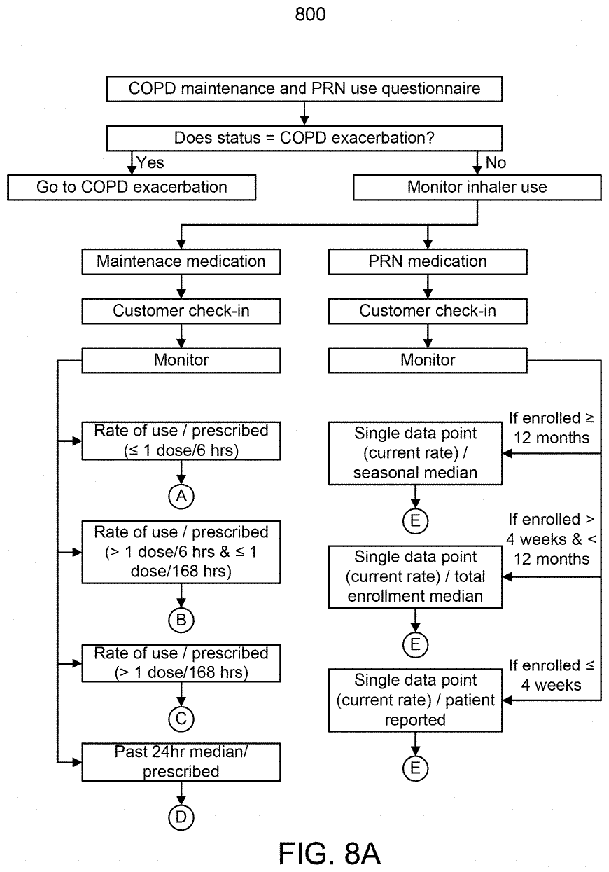

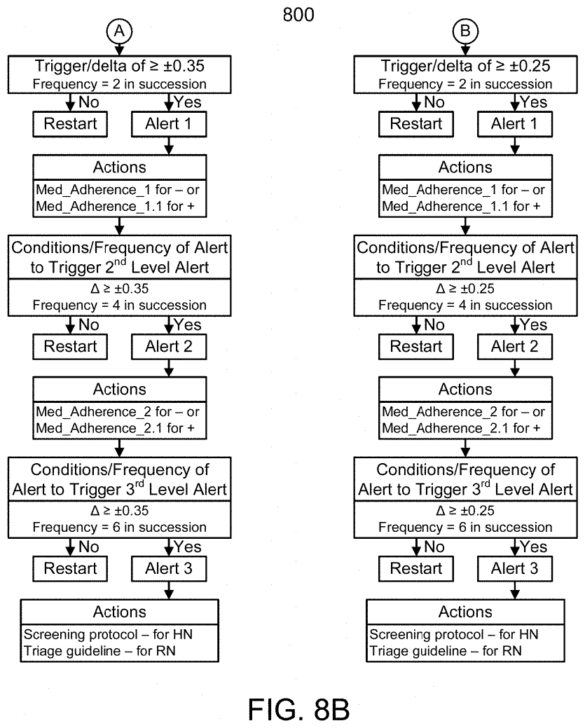

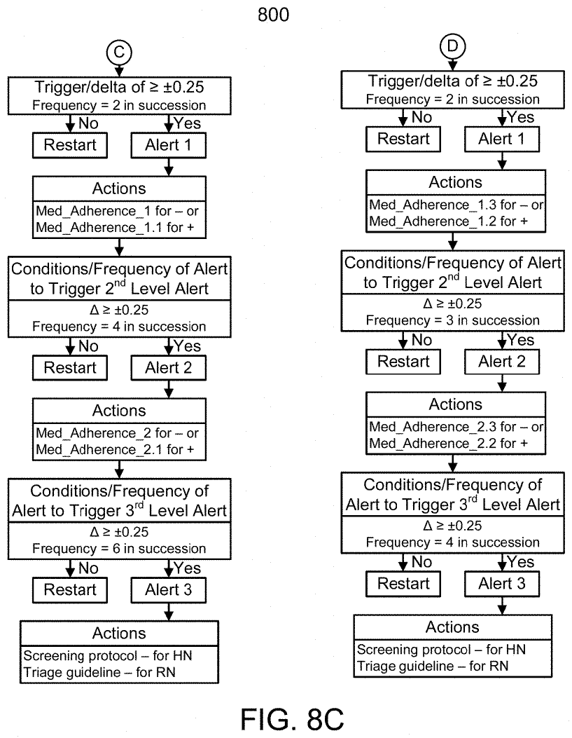

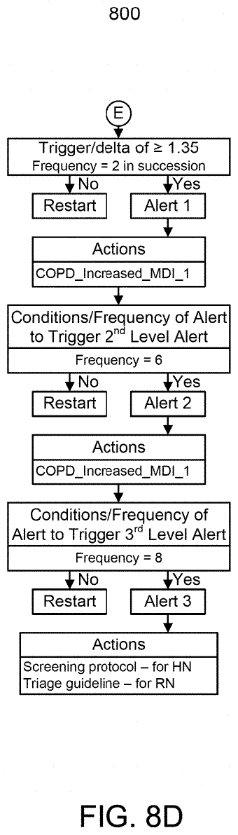

Referring to FIGS. 8A-8D, FIGS. 8A-8D are flow diagrams depicting a method 800 of providing a pathway for COPD maintenance configurable and executable via a pathway engine on a device in accordance with an illustrative embodiment. The functionalities of method 800 may be executed by systems 100 and/or 200 as detailed herein in conjunction with FIGS. 1 and 2. The method 800 may be executed by a pathway for detecting and alerting chronic obstructive pulmonary disease (COPD) maintenance for the user.

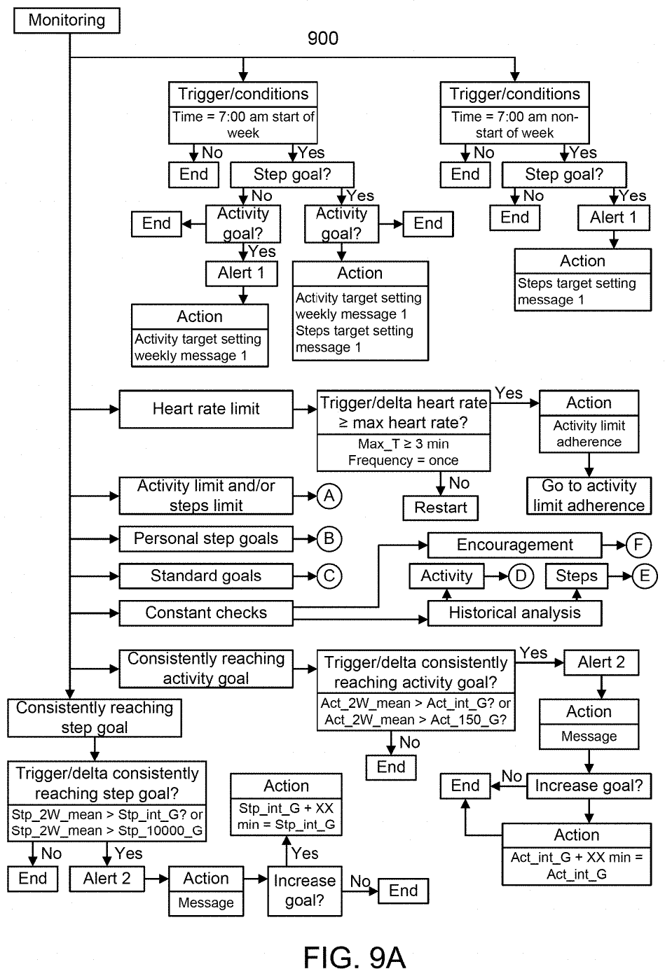

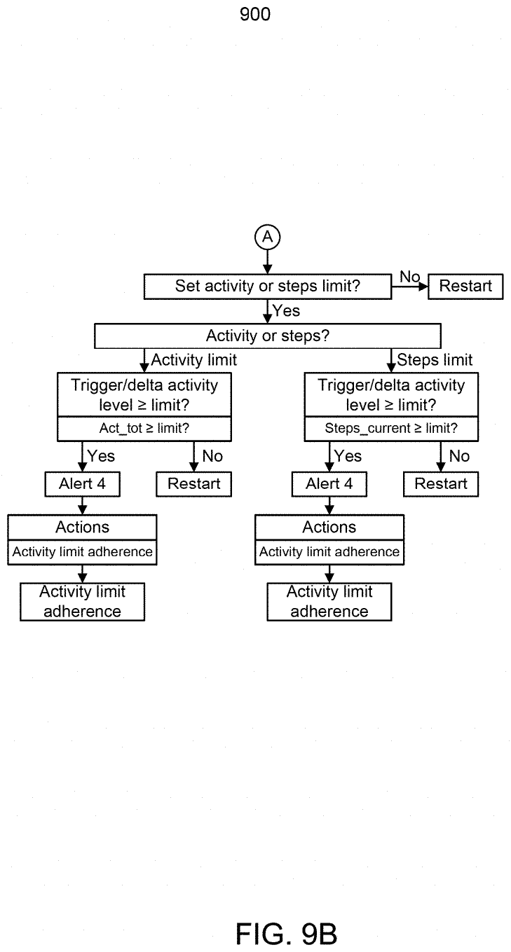

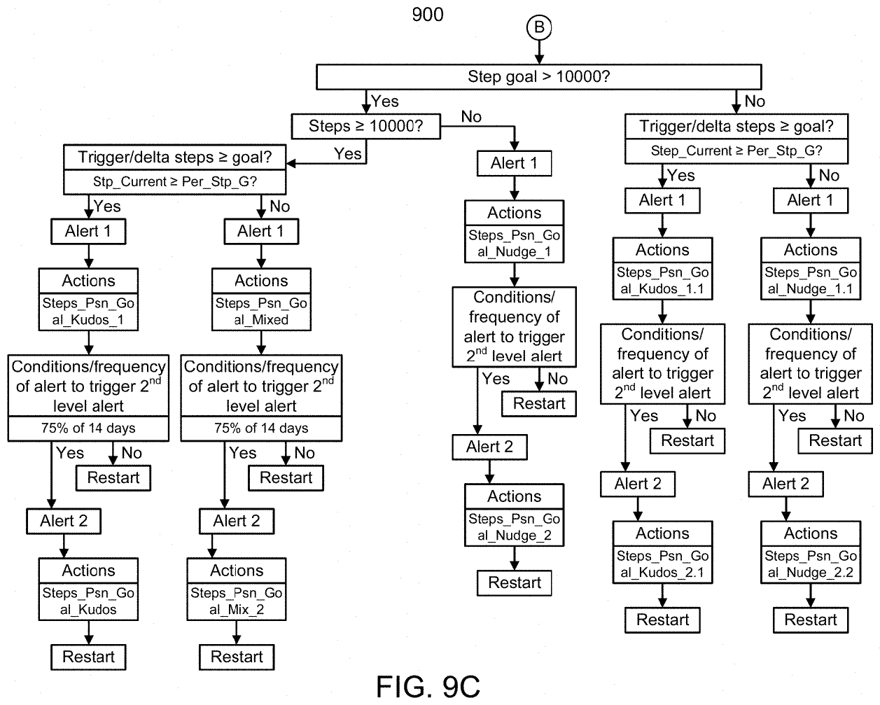

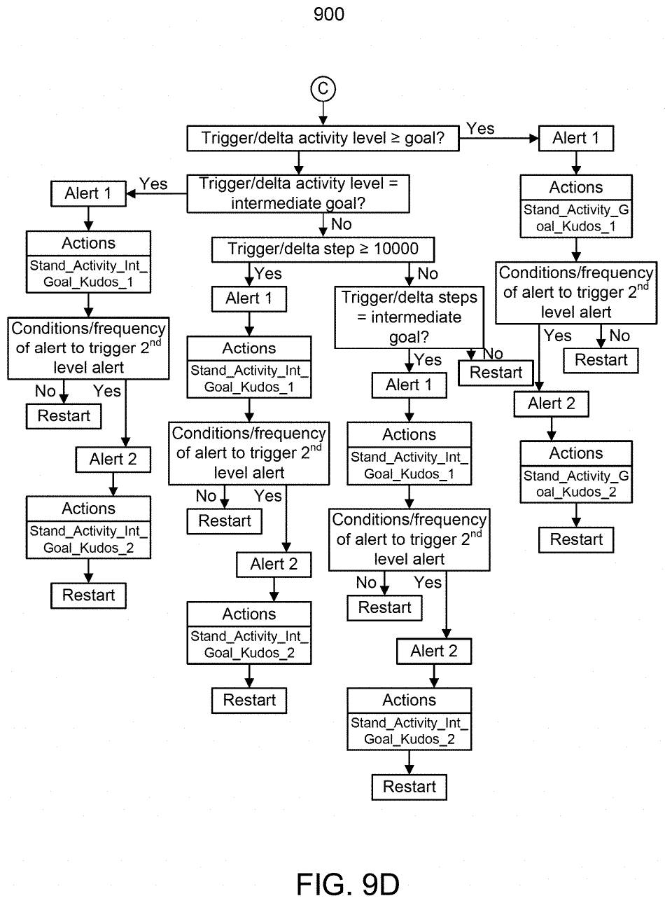

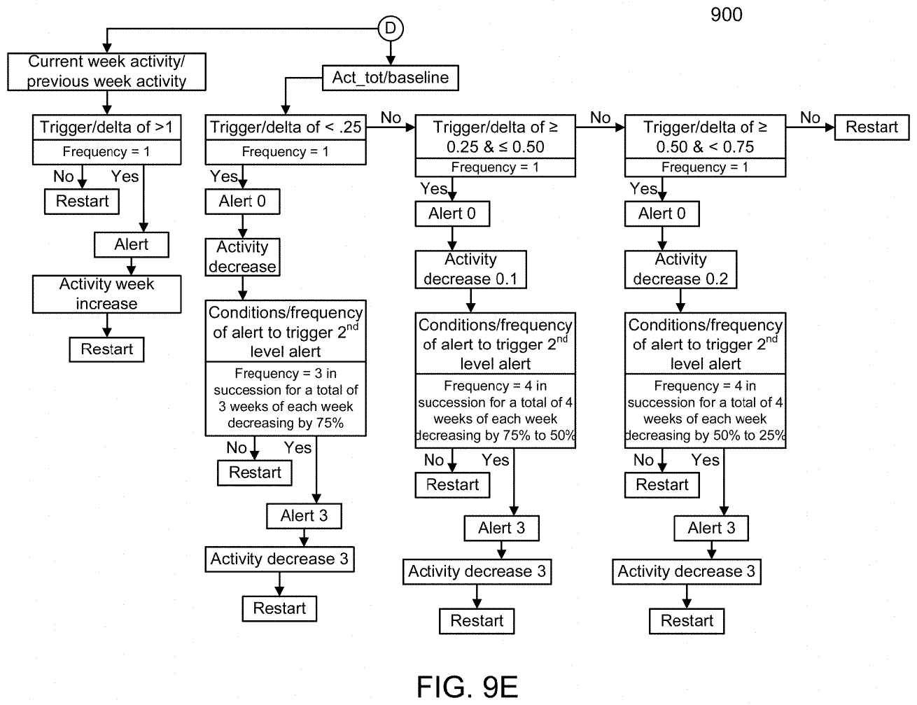

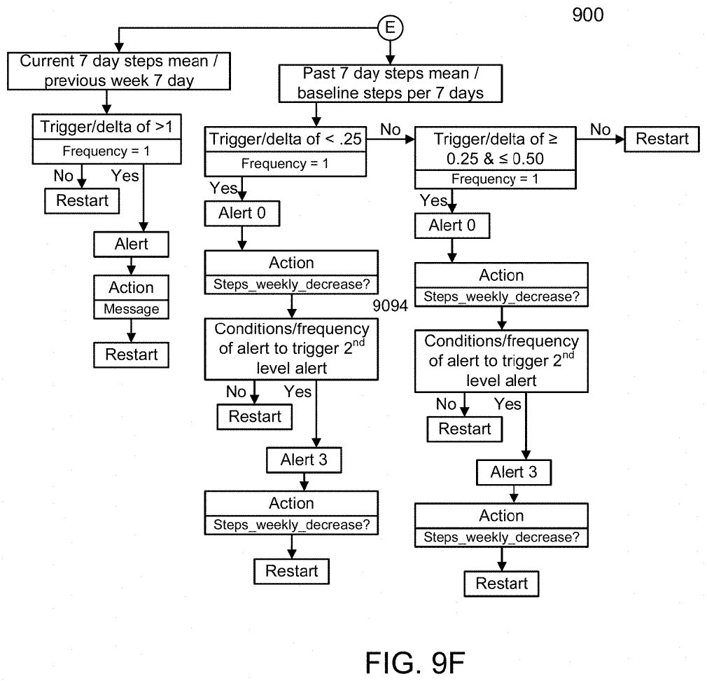

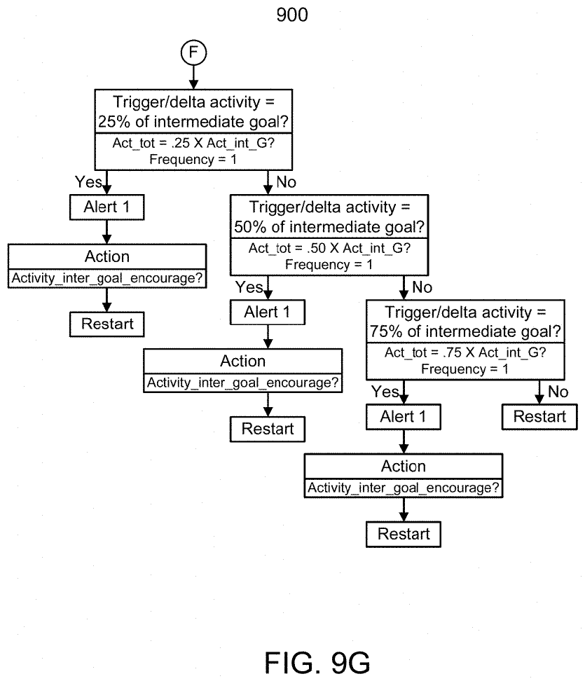

Referring to FIGS. 9A-9G, FIGS. 9A-9G are flow diagrams depicting a method 900 of providing a pathway for activity configurable and executable via a pathway engine on a device in accordance with an illustrative embodiment. The functionalities of method 900 may be executed by systems 100 and/or 200 as detailed herein in conjunction with FIGS. 1 and 2. The method 900 may be executed by a pathway for monitoring physical activity levels in the user.

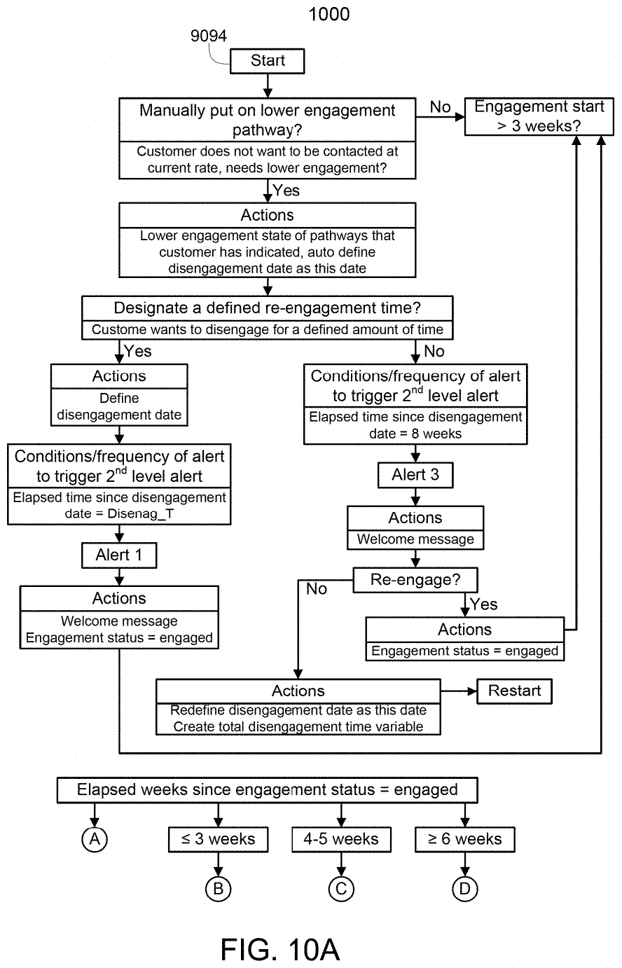

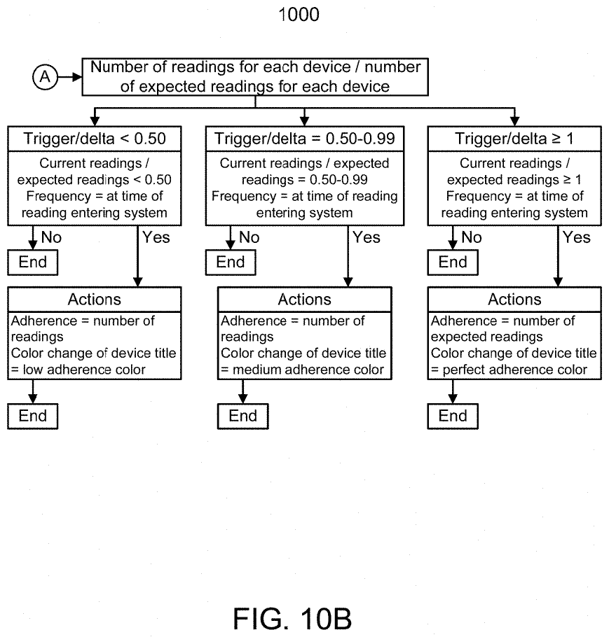

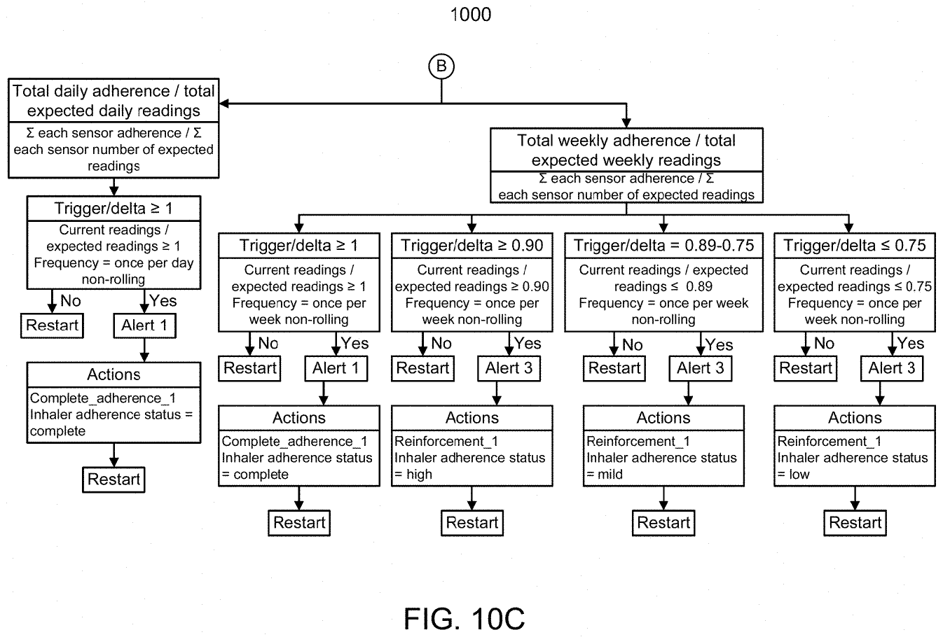

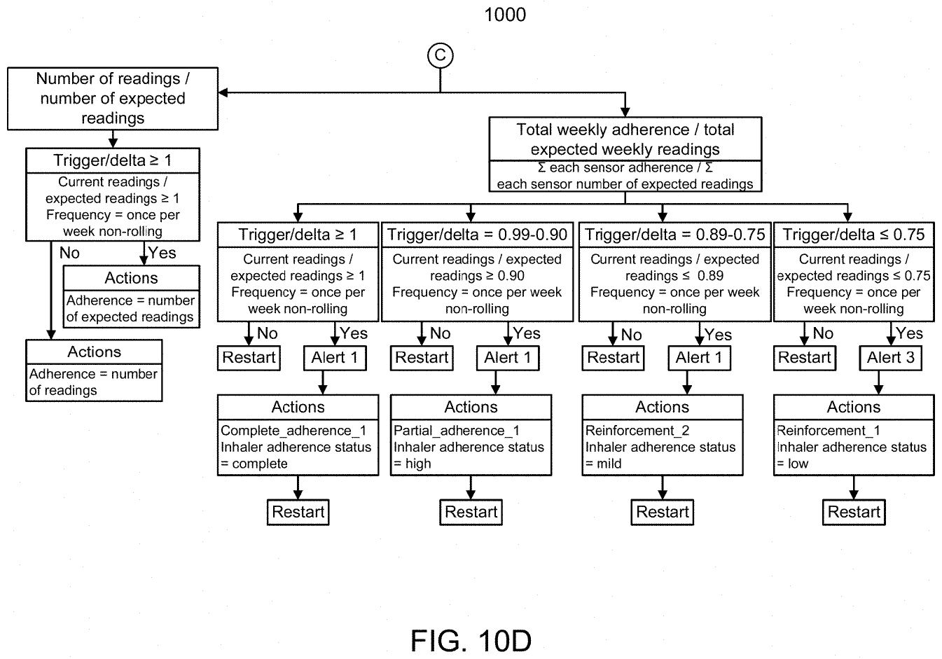

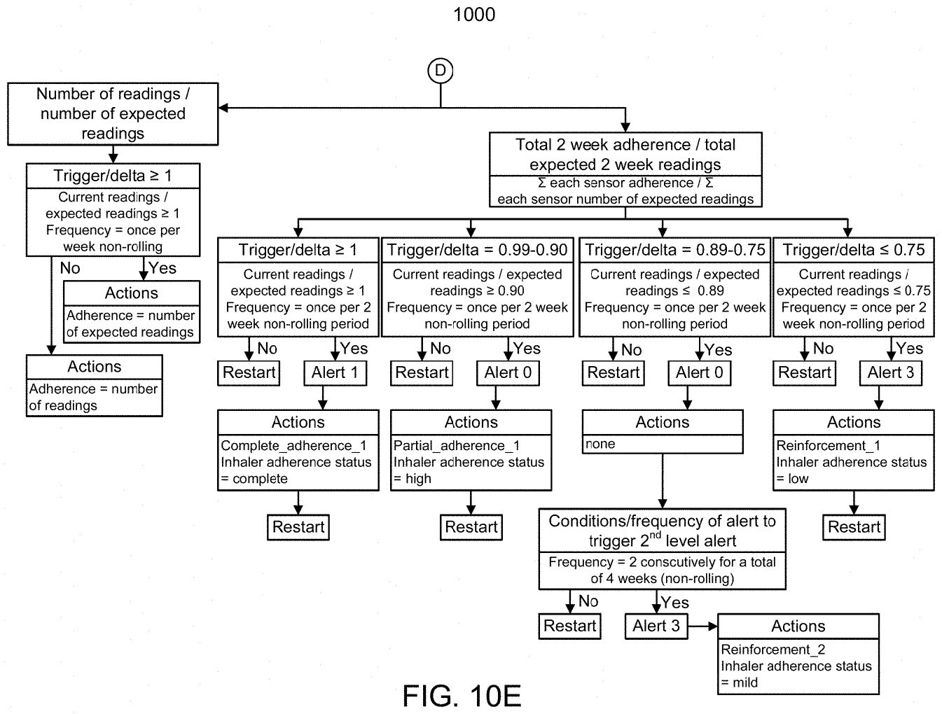

Referring to FIGS. 10A-10E, FIGS. 10A-10E are flow diagrams depicting a method 1000 of providing a pathway for engagement configurable and executable via a pathway engine on a device in accordance with an illustrative embodiment. The functionalities of method 1000 may be executed by systems 100 and/or 200 as detailed herein in conjunction with FIGS. 1 and 2. The method 1000 may be executed by a pathway for physical engagements in the user.

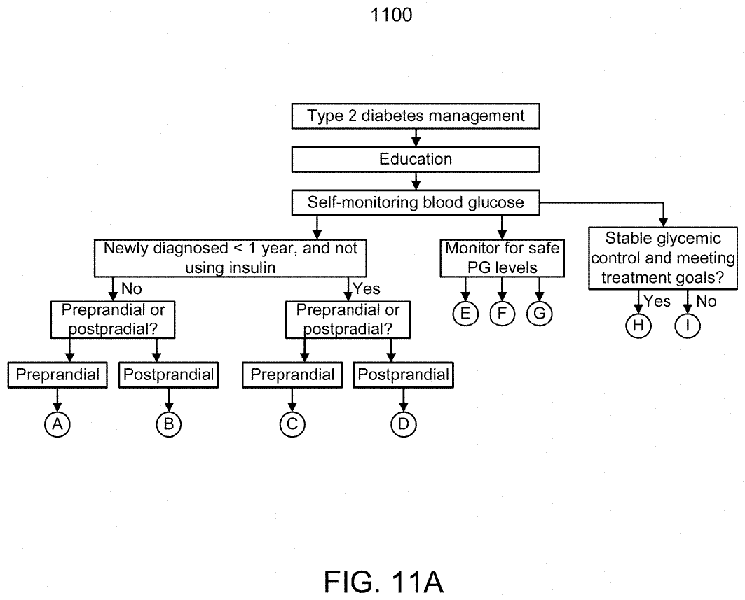

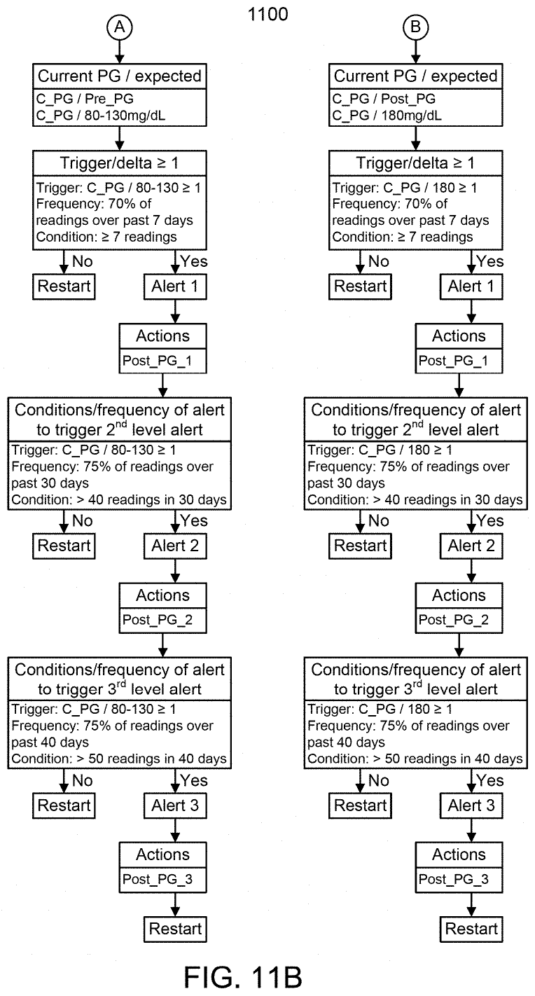

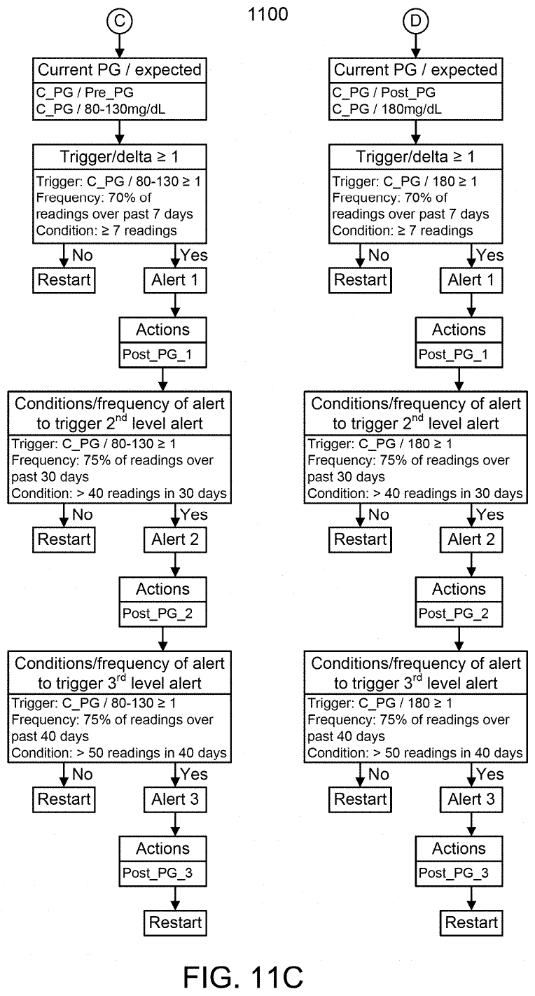

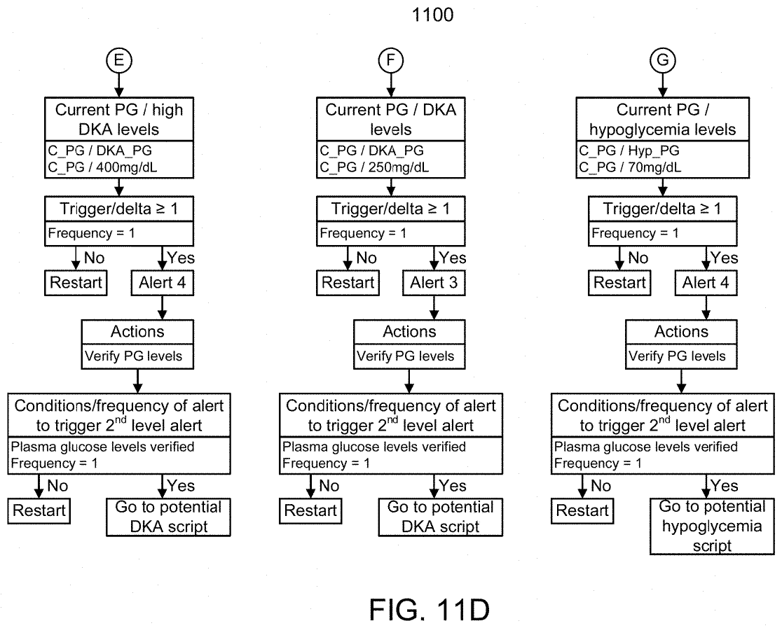

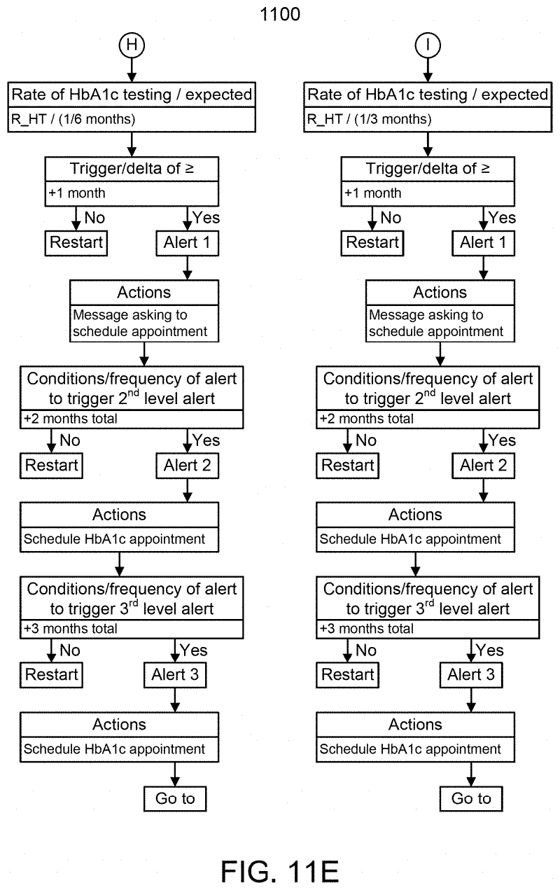

Referring to FIGS. 11A-11E, FIGS. 11A-11E are flow diagrams depicting a method 1100 of providing a pathway for diabetes management configurable and executable via a pathway engine on a device in accordance with an illustrative embodiment. The functionalities of method 1100 may be executed by systems 100 and/or 200 as detailed herein in conjunction with FIGS. 1 and 2. The method 1100 may be executed by a pathway for blood glucose monitoring.

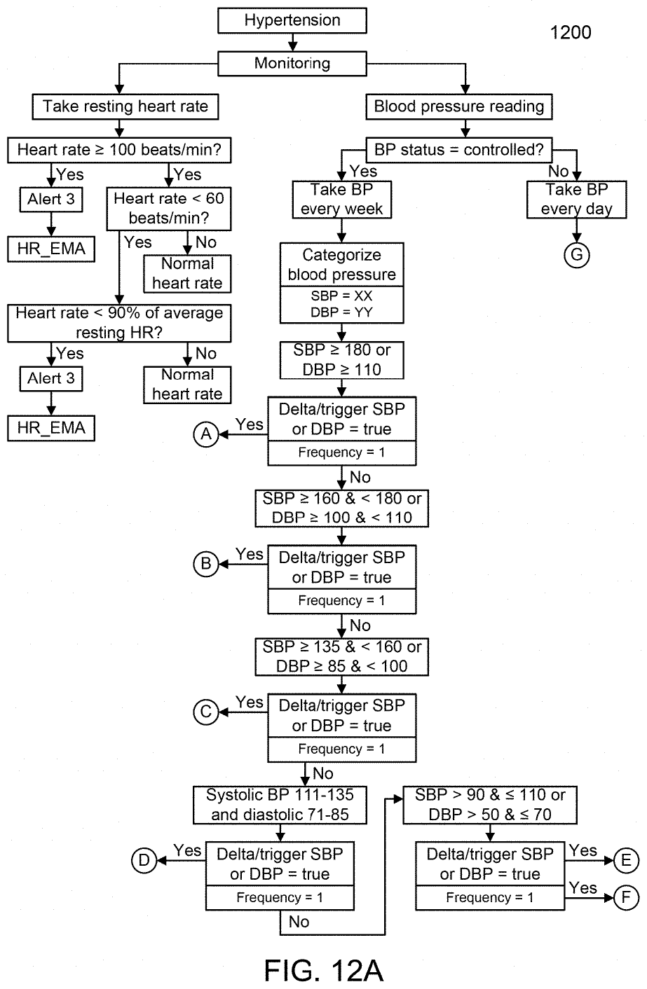

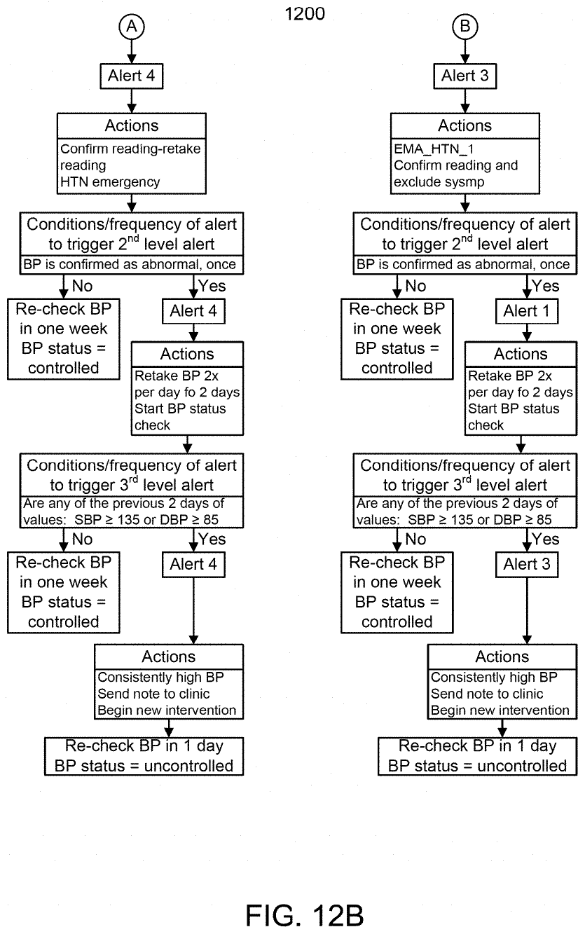

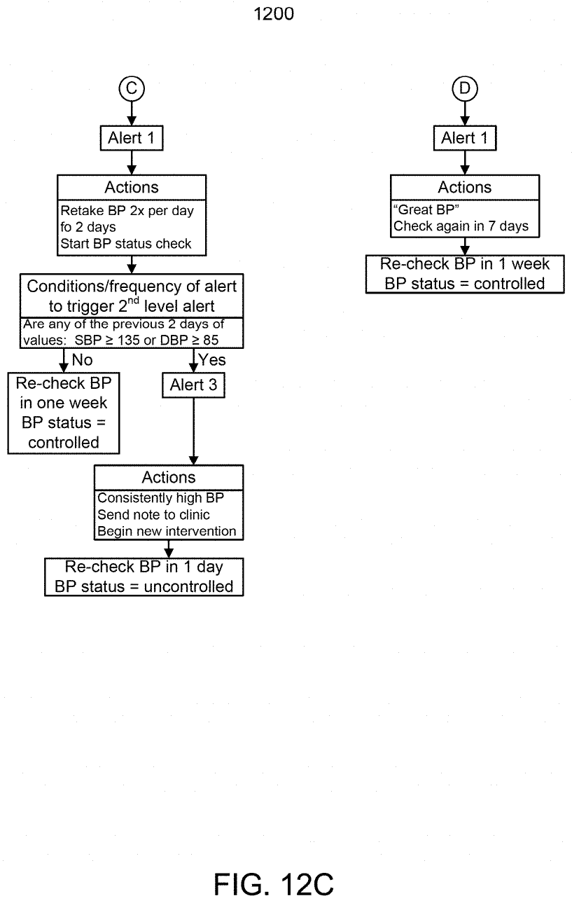

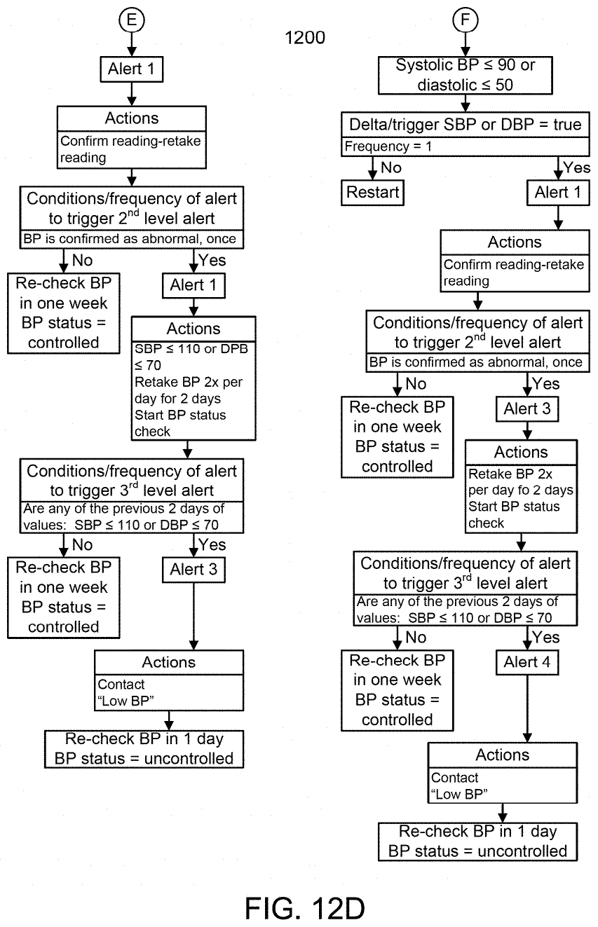

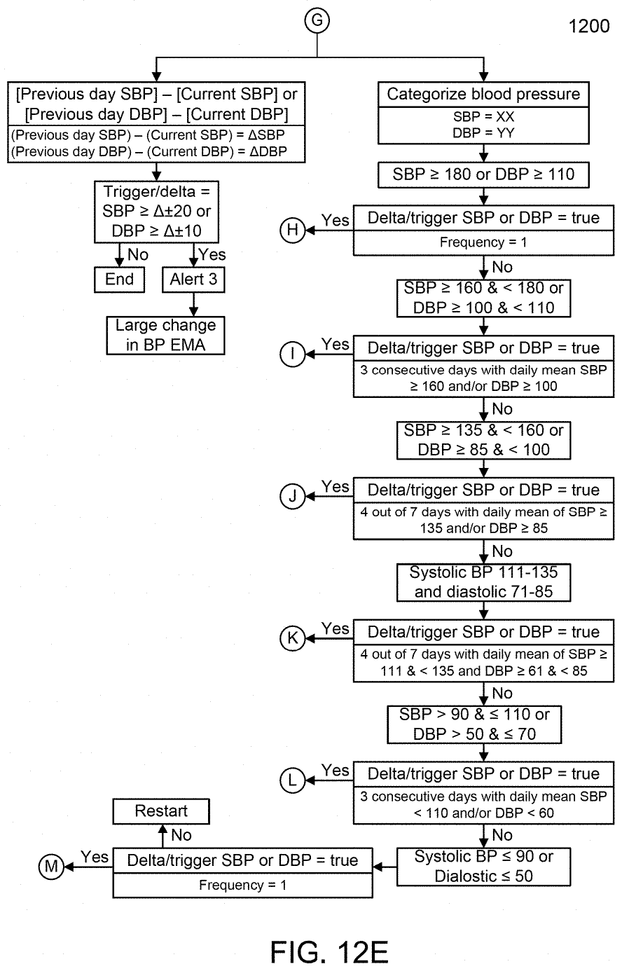

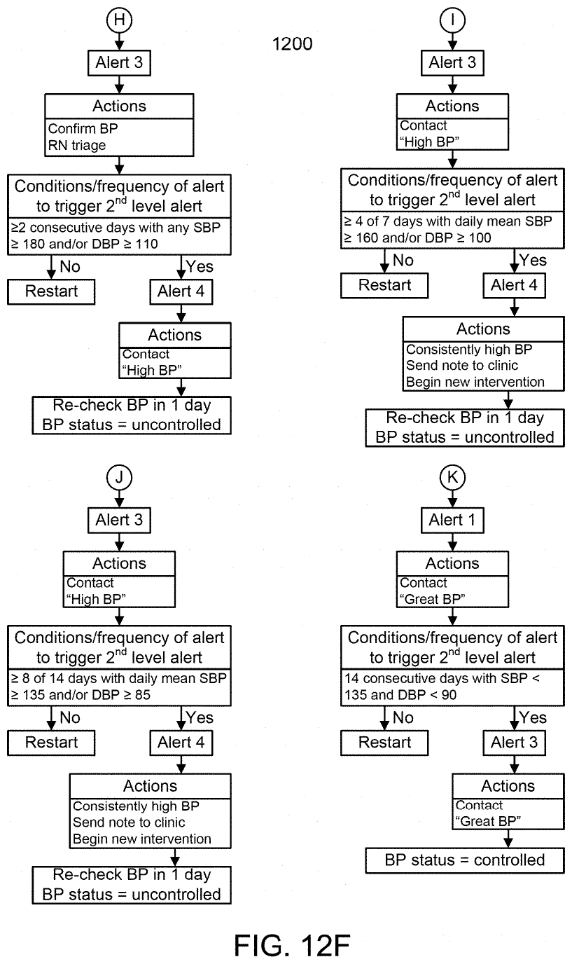

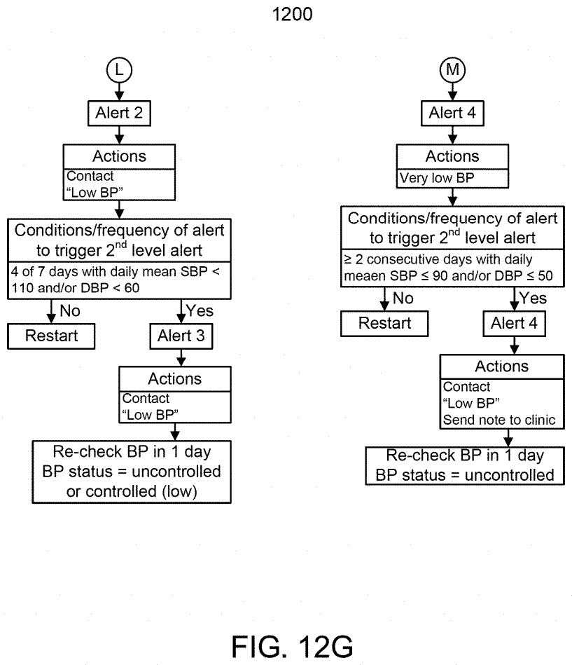

Referring to FIGS. 12A-12G, FIGS. 12A-12G are flow diagrams depicting a method 1200 of providing a pathway for hypertension configurable and executable via a pathway engine on a device in accordance with an illustrative embodiment. The functionalities of method 1200 may be executed by systems 100 and/or 200 as detailed herein in conjunction with FIGS. 1 and 2. The method 1200 may be executed by a pathway for measuring hypertension in the user.

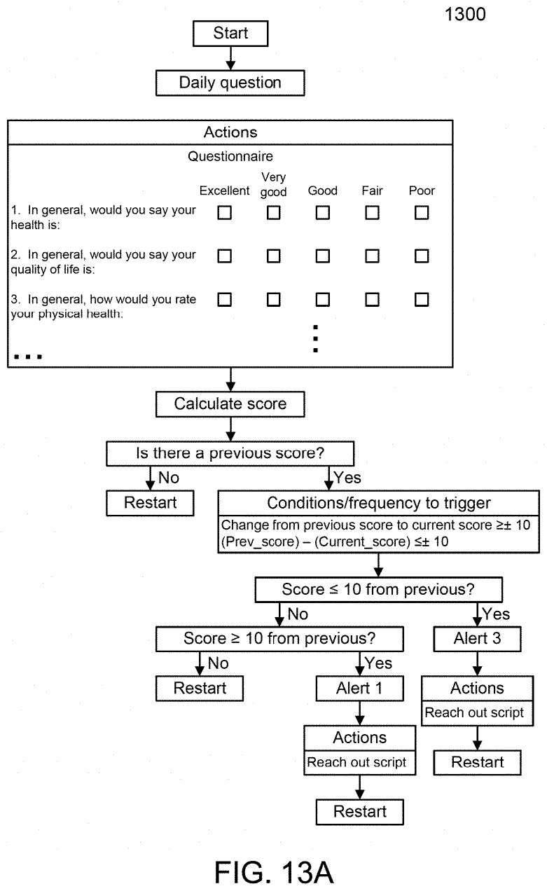

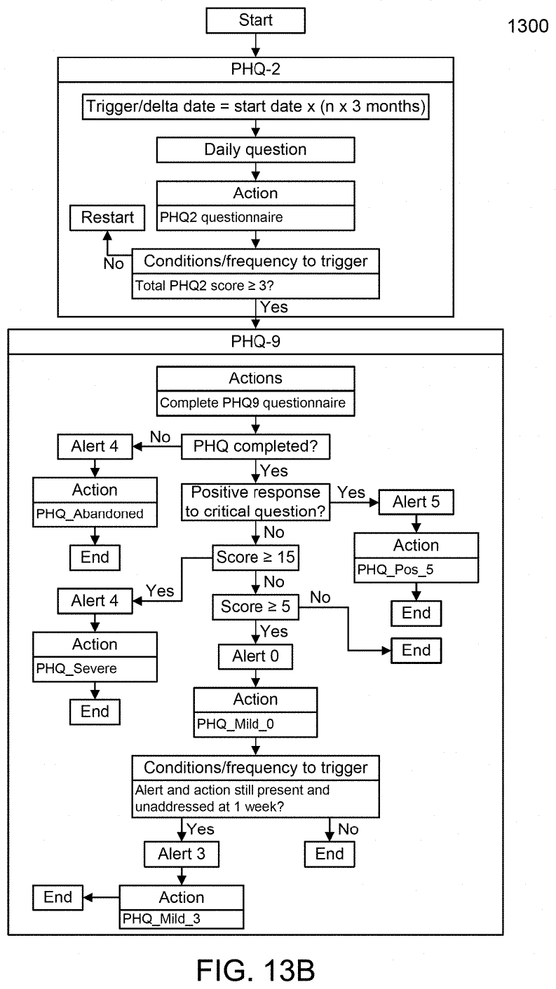

Referring to FIGS. 13A and 13B, FIGS. 13A and 13B are flow diagrams depicting a method 1300 of providing a pathway for hypertension configurable and executable via a pathway engine on a device in accordance with an illustrative embodiment. The functionalities of method 1300 may be executed by systems 100 and/or 200 as detailed herein in conjunction with FIGS. 1 and 2. The method 1300 may be executed by a pathway for patient health questionnaire (PHQ) in the user.

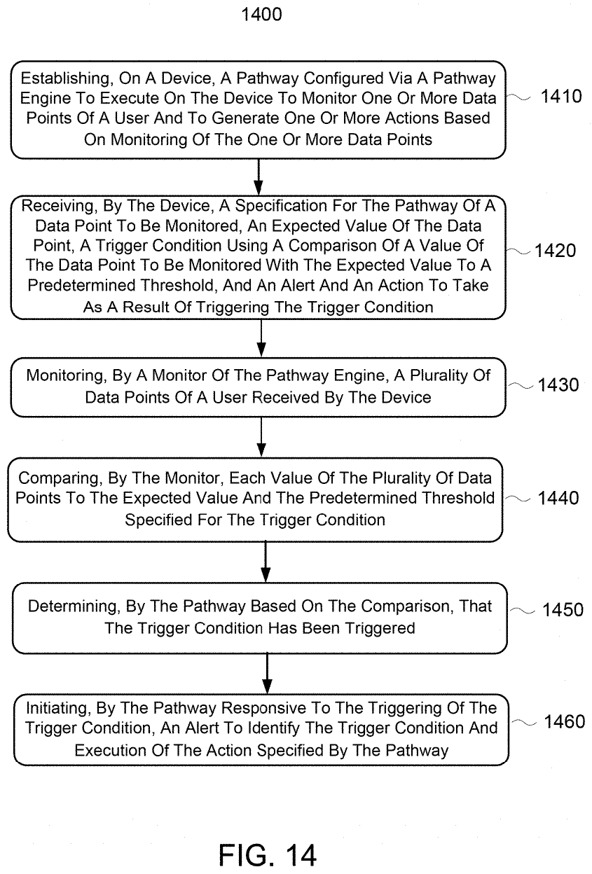

Referring to FIG. 14, FIG. 14 is a flow diagram of a method 1400 for providing a pathway configurable and executable via a pathway engine on a device. The functionalities of method 1400 may be executed by systems 100 and/or 200 as detailed herein in conjunction with FIGS. 1 and 2. In overview, a device may establish a pathway configured via a pathway engine to execute on the device to monitor one or more data points of a user and to generate one or more actions based on monitoring of the one or more data points (BLOCK 1410). The device may receive a specification for the pathway of a data point to be monitored, an expected value of the data point, a trigger condition using a comparison of a value of the data point to be monitored with the expected value to a predetermined threshold, and an alert and an action to take as a result of triggering the trigger condition (BLOCK 1420). A monitor of the pathway engine may monitor a plurality of data points of the user received by the device (BLOCK 1430). The monitor may compare each value of the plurality of data points to the expected value and the predetermined threshold specified for the trigger condition (BLOCK 1440). The pathway, based on the comparison, may determine that the trigger condition has been triggered (BLOCK 1450). The pathway, responsive to the triggering of the trigger condition, may initiate an alert to identify the trigger condition and execution of the action specified by the pathway (BLOCK 1460). Methods 300-1400 may be adopted for use in different contexts, such as cancer treatment, pre/post-surgery, asthma, kidney disease, prescription medication adherence, insensitive treatment, care monitoring, anxiety, stress, depress, and pre-natal monitoring.

The device may establish a pathway configured via a pathway engine to execute on the device to monitor one or more data points of a user and to generate one or more actions based on monitoring of the one or more data points (BLOCK 1410). The pathway may correspond to one of the components in the data sources layer, such as the weight scale, blood pressure cuff, glucometer, activity tracker, ongoing HRA, and the patient reported data, the electronic health record database, claims database, and the other databases at the data sources layer. The device can manage one or more data points received from the pathway of the data sources layer. The device can generate the one or more actions based on the monitoring of the one or more data points from the data sources layer. The one or more actions can include actuating an actuator coupled to the user. The actuator may be part of a device connected to the user, such as that of a medical device at the clinical integration layer. In some embodiments, the device can apply any number of machine learning techniques to determine the one or more actions to take on the patient.

In some embodiments, the device may establish a pathway configured via the device to execute on the device to monitor one or more data points of a user and trigger cascading levels of actions based on monitoring of the one or more data points. The established pathway may correspond to one of the components in the data sources layer, such as the weight scale, blood pressure cuff, glucometer, activity tracker, ongoing HRA, and the patient reported of the sensors 220, the electronic health record database, claims database, and the other databases at the data sources layer. The device of the device may establish bi-directional or unidirectional communications with the pathway configured via the device with the data sources layer. The pathways may use external data, such as weather, environment, social media, socioeconomic, and demographic information regarding the user. The pathways may use data collected by the mobile phone of a patient and devices connected to the mobiles (e.g., Bluetooth-enabled weight-scales, asthma devices, and prescription devices). The pathways may be configurable, adjustable, and dynamic to use different criteria, context, logic, decision points, and interaction points, among others.

The device may receive a specification for the pathway of a data point to be monitored, an expected value of the data point, a trigger condition using a comparison of a value of the data point to be monitored with the expected value to a predetermined threshold, and an alert and an action to take as a result of triggering the trigger condition (BLOCK 1420). The specification for the pathway of the data point to be monitored may include configuration information to process the data point from the pathway. The device may receive an expected value of the data point. The expected value of the data point may correspond to measurements of one of the pathways at the data sources layer. The device may receive a trigger condition using a comparison of a value of the data point to be monitored with the expected value to a predetermined threshold. The device may receive a trigger condition using a comparison of a difference between a value of the data point to be monitored and the expected value, to the predetermined threshold. The trigger condition may be used by the device to execute the designated alert or the action. The device may receive an alert and an action to take as a result of triggering the trigger condition.

A monitor of the pathway engine may monitor a plurality of data points of the user received by the device (BLOCK 1430). The monitor of the device can receive the plurality of data points from one or more components at the data sources layer. The monitor of the device can monitor the data point by receiving data from a sensor measuring the value of the data point. The monitor can receive, for example, weight of the user from the weight scale of the sensors, a blood pressure from the blood pressure cuff of the sensors, a glucose reading from the glucometer of the sensors, an activity reading the activity tracker of the sensors, among others. The device can aggregate the received plurality of data points. The device can store the aggregated plurality of data points by which sensors the respective data points originated. The device can process the received plurality of data points using the various modules of the device, for example, using machine learning, evidence-based care pathways, and alerts, triggers, and rules based on inputs. Machine learning techniques of the device may include artificial neural networks (ANN), nearest neighborhood algorithms (k-NN), reinforcement learning algorithms, Q-learning, and support vector machines (SVMs), among others.

The monitor may compare each value of the plurality of data points to the expected value and the predetermined threshold specified for the trigger condition (BLOCK 1440). The plurality of data points may correspond to data from the one or more components of the data sources layer. The expected value may correspond to a simulated or predicted measurement regarding the user from the respective component at the data sources layer. In some embodiments, the received expected value may be adjusted by the device. The predetermined threshold may represent a degree of deviation from the expected value to satisfy the trigger condition. In some embodiments, the monitor of the device can calculate a difference between the value of the data point and the expected value. In some embodiments, the monitor can determine whether the difference between the value of the data point and the expected value is greater than the threshold.

The pathway, based on the comparison, may determine that the trigger condition has been triggered (BLOCK 1450). In some embodiments, there may be multiple trigger conditions. In such embodiments, the pathway can identify which trigger condition has been triggered based on the comparison. If the difference between the value of the data point and the expected value is greater than the threshold, the pathway can determine that the trigger condition has been triggered. If the difference between the value of the data point and the expected value is less than or equal to the threshold, the pathway can continue monitoring the value of the plurality of data points and comparing each value of the plurality of data points to the expected value and the predetermined threshold specified for the trigger condition.

The pathway, responsive to the triggering of the trigger condition, may initiate an alert to identify the trigger condition and execution of the action specified by the pathway (BLOCK 1460). A type of alert may be specific to the trigger condition and the action. The alert may include a message indicating what action the user should take. A type of action may be specific to the trigger condition and the alert. In some embodiments, responsive to identifying the trigger condition, the pathway can cause the device to instantiate a dialogue window containing the alert. In some embodiments, the pathway can identify the alert corresponding to the trigger condition. In some embodiments, the pathway can determine the alert to present to the user based on the modules of the system intelligence. For example, the pathway can use machine learning techniques to identify the alert to present. In some embodiments, the pathway can relay the alert to the one or more components of the clinical integration layer. In some embodiments, the pathway can identify the action to be executed as specified by the pathway. In some embodiments, the pathway can identify the action to be executed based on the modules of the device. The alert may be used by the device to communicate to the user, upon triggering of the trigger condition. The action may be used by the device to command another client device to actuate in accordance to the command.

In some embodiments, the device may select the action for execution, from a plurality of candidate actions. Which of the plurality of candidate actions is to be selected may be specified by the pathway. The plurality of candidate actions may include actions to be taken by a client device. In some embodiments, the device can determine, from a client device of the user, contextual information pertaining to the user via the one or more monitors of the device. The device can select, using the contextual information, the action for execution from the plurality of candidate actions. The device can relay the selected action to the clinical integration layer.

In some embodiments, the alert may be differentiated by levels. The pathway can initiate, responsive to the triggering of the trigger condition, the first level alert to identify the trigger condition and execution of the first action specified by the pathway. The first level alert may correspond to an initial alert to the user. For example, the first level alert may correspond to an initial onset of a certain health symptoms that the user may have. First action may correspond to an initial action to be taken by the user. For example, the first level action may correspond to an preliminary action to help offset such health symptoms that the user may have. In addition, the monitor can monitor, responsive to triggering of the trigger condition, values of the plurality of data points for one of the condition of the user or frequency of triggering the trigger condition upon which to trigger the second level alert and the second action.

Responsive to executing the action, the device can adjust the expected value of the data point based on the action. In some embodiments, the device may adjust an expected value of a second data point of the pathway based on the action, the second data point of the user identified in the specification for the pathway as dependent on the first data point and the action, responsive to executing the action. In some embodiments, the pathway can calculate an adjustment to the expected value based on the action using the modules of the system intelligence. For example, the pathway can estimate the adjustment to the expected value using machine learning techniques and the action taken. If the user is asked to exercise in response to an alert from a chronic health failure pathway, the pathway can estimate that the likelihood of the user of suffering from such health failure to decrease by a preset percentage.

### C. Systems and Methods for Providing Interoperability Between Pathways

By providing interoperability between pathways, the system 200 may use data from various pathways to process and infer an outcome in one pathway. While each pathway may monitor a defined set of parameters and creates actions based on those data points, each pathway may influence each other at clinically relevant points. The complex interaction of pathways with each other may be described as a single large unit or system. Each part of that system can function independently but becomes increasingly enriched with inputs from other pathways. Compiling the data points may a more complete view of an individual's health and wellness in the context of their life.

Still referring to FIG. 2, to provide for interoperability between various pathways, the device may execute, via the pathway engine 228, a plurality of different pathways. Each pathway of the plurality of different pathways may monitor a defined set of data points of a user and execute actions based on monitoring of the defined set of data points. In some embodiments, the device may identify multiple subsets of different pathways to monitor for the respective defined set of data points of the users and execute actions based on monitoring of the defined set of data points.

The device may maintain a model for the plurality of different pathways based on the defined set of data points for the user from the plurality of different pathways. In some embodiments, the device may maintain a plurality of models for the plurality of different pathways based on the defined set of data points for the user from the plurality of different pathways. Each model may be trained using the training data from the data source layer 205 (e.g., electrical health records database 222). Each model may be used to determine a relevancy score or a correlation metric of the data from one pathway to another pathway. In some embodiments, one model maintained by the device may be for a particular pathway and another model maintained by the device may be for the plurality of different pathways. In some embodiments, the device may store the model at one of the modules of the system intelligence 232. As the device receives more data from the plurality of different pathways, the device may update the model based on the newly received data.

A first pathway of the plurality of different pathways may determine, responsive to monitoring values of a first defined set of data points of the user, a point in execution of the first pathway to receive one or more inputs from a second pathway of the plurality of different pathways. In some embodiments, the first pathway can set the point in execution in accordance with a data transfer schedule. The data transfer schedule can specify a time at which to execute the first pathway to receive the one or more inputs from the second pathway. The time may be an interval (e.g., once every 30 minutes, week, etc.) or at random. In some embodiments, the first pathway may generate the data transfer schedule in accordance to the model maintained by the device. In some embodiments, the data transfer schedule may be pre-set by a system administrator. In some embodiments, the data transfer schedule may be pre-set by one of the various components at the clinical integration layer 215.

The first pathway may receive, from the second pathway, as input one or more data values of a second defined set of data being monitored by the second pathway. In some embodiments, the first pathway may select the second pathway for the input based on a pathway selection policy. The pathway selection policy may specify which of the plurality of different pathways to select as inputs for the first pathway. For example, some pathways may be more relevant than other pathways. In some embodiments, the first pathway may generate the pathway selection policy in accordance to the model maintained by the device. The model may indicate which of the plurality of pathways are relevant to the first pathway. In some embodiments, the pathway selection policy may be pre-set by a system administrator. In some embodiments, the pathway selection policy may be pre-set by one of the various components at the clinical integration layer 215.

In some embodiments, the first pathway may determine or calculate a relevancy score for the defined set of data points to the first pathway based on the model maintained by the device. The relevancy score may indicate the relevance or inter-correlation between the set of data points from another pathway (e.g., the second pathway) of the plurality of pathways to the first pathway. In some embodiments, the first pathway may determine or calculate the relevancy score based on the model maintained by the device. The first pathway may compare the relevancy score to a threshold. If the relevancy score is greater than the threshold, the first pathway may select the data points from the second pathway as the inputs for the first pathway. In some embodiments, the first pathway may weigh the relevancy score based on the pathway selected.

The first pathway may determine, using at least the values of the first defined set of data points and one or more values of the second defined set of data, to take a predetermined action of the first pathway. The relevancy score may indicate the relevance or inter-correlation between the set of data points from another pathway (e.g., the second pathway) of the plurality of pathways to the first pathway. In some embodiments, the first pathway may compare the calculated relevancy score to a relevancy score threshold for the first pathway and the second pathway. In some embodiments, the first pathway may take the predetermined action of the first pathway, responsive to determining that the relevancy score is greater than the relevancy score threshold. The predetermined action may include actuating an actuator or a client device at the clinical integration layer 215. In some embodiments, the device may select the predetermined action from a plurality of candidate actions to take for the first pathway based on the values of the defined set of data points.

In some embodiments, the device can generate a metric based on the values of the defined set of data points of the use for each of the plurality of different pathways. The metric may be indicative of the relevance or inter-correlation between the data points of the plurality of different pathways. In some embodiments, the device can generate the metric based on the model maintained by the device. In some embodiments, the device can generate a first metric based on the values of the first defined set of data points for the first pathway. In some embodiments, the device can generate a second metric based on the values of the second defined set of data points for the second pathway. Using the generated metric, the first pathway can take the predetermined action of the first pathway.

In some embodiments, the device can determine a correlation metric between the value of defined sets of data points from the plurality of different pathways. The correlation metric may be indicative of the cross-correlation between the data points of the plurality of different pathways. For example, data from the pathway for sleep may be correlated with data from the pathway for chronic heart failures. In some embodiments, the device can generate the correlation metric based on the model maintained by the device. The device can compare the correlation metric to a correlation threshold for the pathways used to generate the correlation metric. The correlation threshold may be pre-set or dynamically calculated using the modules of the system intelligence 232. In some embodiments, the first pathway may take the predetermined action, responsive to determining that the correlation metric is greater than the correlation threshold.

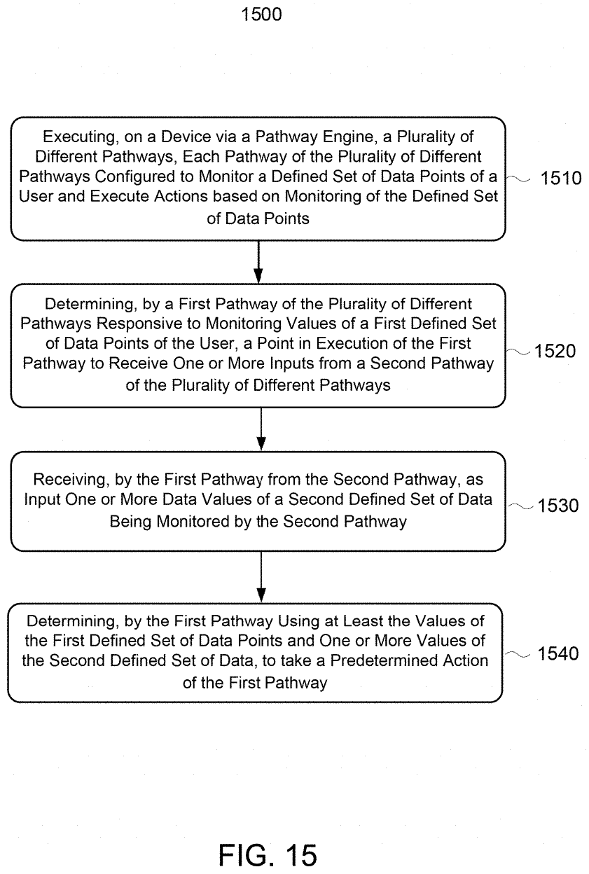

Referring now to FIG. 15, FIG. 15 is a flow diagram of a method 1500 for interoperability between pathways configured and executable on a device. The functionality of method 1500 may be executed by the systems 100 and/or 200 as detailed herein in conjunction with FIGS. 1 and 2. In overview, the device can execute, via a pathway engine, a plurality of different pathways (BLOCK 1505). Each pathway of the plurality of different pathways may monitor a defined set of data points of a user and execute actions based on monitoring of the defined set of data points. A first pathway of the plurality of different pathways may determine, responsive to monitoring values of a first defined set of data points of the user, a point in execution of the first pathway to receive one or more inputs from a second pathway of the plurality of different pathways (BLOCK 1510). The first pathway may receive, from the second pathway, as input one or more data values of a second defined set of data being monitored by the second pathway (BLOCK 1515). The first pathway may determine, using at least the values of the first defined set of data points and one or more values of the second defined set of data, to take a predetermined action of the first pathway (BLOCK 1520).

The device can execute, via a pathway engine, a plurality of different pathways (BLOCK 1505). Each pathway of the plurality of different pathways may monitor a defined set of data points of a user and execute actions based on monitoring of the defined set of data points. In some embodiments, the device may identify multiple subsets of different pathways to monitor for the respective defined set of data points of the users and execute actions based on monitoring of the defined set of data points. The device may maintain a model for the plurality of different pathways based on the defined set of data points for the user from the plurality of different pathways. In some embodiments, the device may maintain a plurality of models for the plurality of different pathways based on the defined set of data points for the user from the plurality of different pathways. Each model may be trained using the training data from the data source layer (e.g., electrical health records database). Each model may be used to determine a relevancy score or a correlation metric of the data from one pathway to another pathway. In some embodiments, one model maintained by the device may be for a particular pathway and another model maintained by the device may be for the plurality of different pathways. In some embodiments, the device may store the model at one of the device. As the device receives more data from the plurality of different pathways, the device may update the model based on the newly received data.

A first pathway of the plurality of different pathways may determine, responsive to monitoring values of a first defined set of data points of the user, a point in execution of the first pathway to receive one or more inputs from a second pathway of the plurality of different pathways (BLOCK 1510). In some embodiments, the first pathway can set the point in execution in accordance with a data transfer schedule. The data transfer schedule can specify a time at which to execute the first pathway to receive the one or more inputs from the second pathway. The time may be an interval or at random. In some embodiments, the first pathway may generate the data transfer schedule in accordance to the model maintained by the device. In some embodiments, the data transfer schedule may be pre-set by a system administrator. In some embodiments, the data transfer schedule may be pre-set by one of the various components at the clinical integration layer.

The first pathway may receive, from the second pathway, as input one or more data values of a second defined set of data being monitored by the second pathway (BLOCK 1515). In some embodiments, the first pathway may select the second pathway for the input based on a pathway selection policy. The pathway selection policy may specify which of the plurality of different pathways to select as inputs for the first pathway. For example, some pathways may be more relevant than other pathways. In some embodiments, the first pathway may generate the pathway selection policy in accordance to the model maintained by the device. The model may indicate which of the plurality of pathways are relevant to the first pathway. In some embodiments, the pathway selection policy may be pre-set by a system administrator. In some embodiments, the pathway selection policy may be pre-set by one of the various components at the clinical integration layer.

In some embodiments, the first pathway may determine or calculate a relevancy score for the defined set of data points to the first pathway based on the model maintained by the device. The relevancy score may indicate the relevance or inter-correlation between the set of data points from another pathway (e.g., the second pathway) of the plurality of pathways to the first pathway. In some embodiments, the first pathway may determine or calculate the relevancy score based on the model maintained by the device. The first pathway may compare the relevancy score to a threshold. If the relevancy score is greater than the threshold, the first pathway may select the data points from the second pathway as the inputs for the first pathway. In some embodiments, the first pathway may weigh the relevancy score based on the pathway selected.

The first pathway may determine, using at least the values of the first defined set of data points and one or more values of the second defined set of data, to take a predetermined action of the first pathway (BLOCK 1520). The first pathway may calculate a relevancy score using the values of the first defined set of data points and the one or more values of the second defined set of data. The relevancy score may indicate the relevance or inter-correlation between the set of data points from another pathway (e.g., the second pathway) of the plurality of pathways to the first pathway. In some embodiments, the first pathway may compare the calculated relevancy score to a relevancy score threshold for the first pathway and the second pathway. In some embodiments, the first pathway may take the predetermined action of the first pathway, responsive to determining that the relevancy score is greater than the relevancy score threshold. The predetermined action may include actuating an actuator or a client device at the clinical integration layer. In some embodiments, the device may select the predetermined action from a plurality of candidate actions to take for the first pathway based on the values of the defined set of data points.

In some embodiments, the device can generate a metric based on the values of the defined set of data points of the use for each of the plurality of different pathways. The metric may be indicative of the relevance or inter-correlation between the data points of the plurality of different pathways. In some embodiments, the device can generate the metric based on the model maintained by the device. In some embodiments, the device can generate a first metric based on the values of the first defined set of data points for the first pathway. In some embodiments, the device can generate a second metric based on the values of the second defined set of data points for the second pathway. Using the generated metric, the first pathway can take the predetermined action of the first pathway.

In some embodiments, the device can determine a correlation metric between the value of defined sets of data points from the plurality of different pathways. The correlation metric may be indicative of the cross-correlation between the data points of the plurality of different pathways. For example, data from the pathway for sleep may be correlated with data from the pathway for chronic heart failures. In some embodiments, the device can generate the correlation metric based on the model maintained by the device. The device can compare the correlation metric to a correlation threshold for the pathways used to generate the correlation metric. The correlation threshold may be pre-set or dynamically calculated. In some embodiments, the first pathway may take the predetermined action, responsive to determining that the correlation metric is greater than the correlation threshold.

### D. Systems and Methods for Interjecting a Communication to a User in a Point of Execution of a Pathway

By interjecting communications to a user, the system may relay alerts, health treatment plans, and messages, among others to the user. Referring back to FIG. 2, in some embodiments, the device provides functional and interactive features to customers (e.g., in a mobile app on a smartphone). The device may provide a user experience and user interface, a design, a business process of data movement to allow for upstream and proactive health care, or the like. In some embodiments, the customers may utilize the device via a user interface to provide health-related inputs (e.g., via sensors) to the system for analysis by the system, and the system may provide continuous health risk assessments based on the data inputs (e.g., from the sensors). In some arrangements, these health assessments take the form of health messages or alerts that are delivered a smart device, or computing device of the user. In some embodiments, the messages are customized using human curated communication segmentation sets, so that support, encouragement, recommendations for further action, among others are provided to the user in a highly personalized way taking into account for example, user age, user location, user interests, user device use, sensor inputs, previous inputs into the device, among others. In various embodiments, these personalized messages may be delivered to web applications, smart devices, over phone, or via email or text messages.

A device may, via a pathway engine, execute a pathway configured to monitor a defined set of data points of a user and execute one or more actions based on monitoring the defined set of data points. A monitor of the pathway engine may monitor a plurality of data points of a user received by the device. The pathway may, responsive to monitoring values of the defined set of data points of a user, determine a point in execution in which to communicate to the user. The pathway may select, from a plurality of different communication schemes, a communication scheme for communicating to the user. The plurality of communication schemes may include at least one of the following: a text message to the mobile phone of the user, a push notification to an application on the mobile phone of the user, a telephone call to one or more telephone numbers of the user, a chat session with the user, and a video session with the user.

The pathway may initiate the selected communication scheme. The selected communication scheme may be initiated by, for example, transmitting a message to a smart device associated with the user, initiating a phone call with the user, or the like.

In some embodiments, executing the pathway may further include receiving, by the device, a specification for the pathway of a data point to be monitored, an expected value of the data point, a trigger condition using a comparison of a value of the data point to be monitored with the expected value to a predetermined threshold, and an alert and an action to take as a result of triggering the trigger condition. In some embodiments, determining the point in execution may further include determining the point in execution in which to communicate to the user based on the specification for the pathway as the result of triggering the trigger condition. For example, when triggering condition is triggered, the device may transmit the alert, along with the action to take, to a smart device associated with the user. In some embodiments, selecting the communication scheme may further include selecting the communication scheme based on the specification for the pathway as the result of triggering the trigger condition. In some embodiments, selecting the communication scheme may further include measuring the severity of the triggering condition. For example, if the device detects that the monitored data is indicative of a particularly severe medical condition, the device may select a communication scheme that includes establishing a connection between the user and a health navigator. In some embodiments, establishing a connection between the user and a health navigator may include alerting a health navigator to contact the customer by way of a phone call, chat session, text message, or the like.

In some embodiments, initiating the selected communication scheme may further include transmitting an automated message of the selected communication scheme to a smart device associated with the user. In some embodiments, the transmitted message includes an alert that notifies the customer of the triggering of the trigger condition. In some embodiments, the transmitted message also includes a recommended action that the user should take as a result of the triggering of the triggering condition. In some embodiments, the warning message may include queries that request responses from the user concerning the health of the user.

In some embodiments, determining the point in execution may further include determining the point in the execution in which to communicate to the user in accordance with a communication schedule, the communication schedule specifying a time at which to initiate the selected communication scheme for communicating to the user. For example, when the specified time is reached, the form of communication designated by the selected communication scheme may be transmitted to the user. In some embodiments, determining the point in execution may further include determining the point in execution in which to communicate to the user, responsive to monitoring second values of a second defined set of data points of the user from second pathway different from the pathway. In some embodiments, determining the point in execution may further include receiving, from the second pathway, an indicator specifying the point in execution in which to communicate to the user, the indicator generated by the second pathway responsive to a specification for the second pathway.

In some embodiments, selecting the communication scheme may further include transmitting a request to establish communications between a navigator and the user, responsive to determining the point in the execution to communicate to the user. The request to establish communications between a navigator and the user may include, for example, a telephone call to a smart device of the user, a chat session, or the like. In some embodiments, the request to establish communications between a navigator and the user may be transmitted after sending the user an automated message or alert. For example, the system may first transmit the user with an automated message that provides the user with questions to answer. In some embodiments, the automated message takes the form of a graphical interface that is presented on a smart device of the user. The graphical interface may give the user the ability to indicate whether various symptoms are applicable to them, or provide the user with the ability to input a description of how the user currently feels. In some arrangements, selecting the communication scheme may further include receiving user responses to the presented questions, and determining the severity of the health condition of the user. In response to certain customer responses to the presented questions, tending to indicate a more severe medical scenario, the system may then transmit the request to establish communications between a navigator and the user responsive to the received user responses.

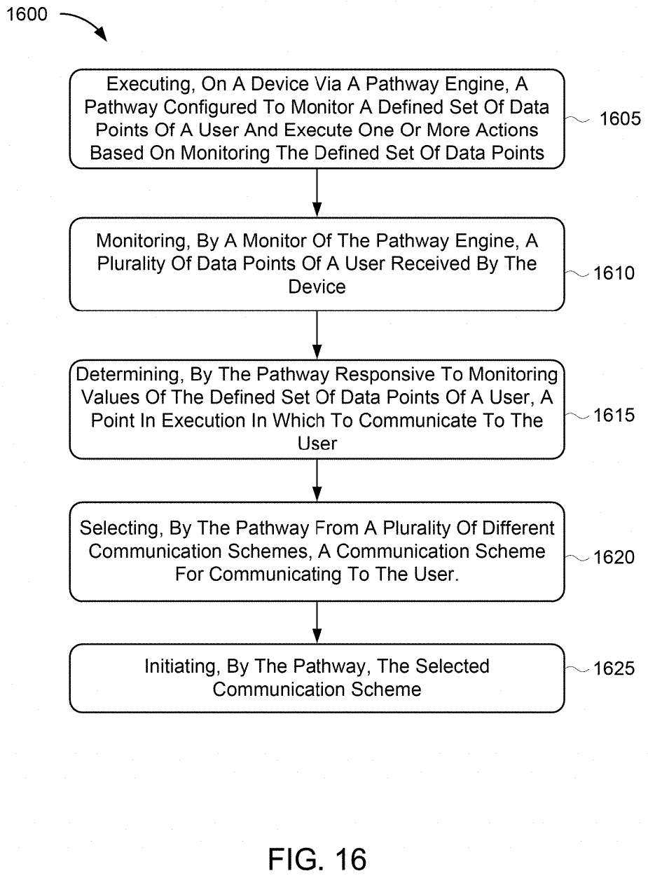

Referring now to FIG. 16, a flow diagram depicting a method 1600 for interjecting a communication to a user in a point of execution of a pathway configurable and executable via a pathway engine on a device is shown according to an illustrative embodiment. The functionality described herein with respect to method 1600 can be performed or otherwise executed by the systems discussed in relation to the environment 200 as shown in FIG. 2, or the computing devices 100 shown in FIG. 1C or 1D, or any combination thereof.

In further detail, the computing device can execute a pathway (BLOCK 1605). In some embodiments, the computing device may execute the pathway via a pathway engine that is stored in a storage unit of the computing device. The pathway may identify a set of defined data points to monitor in relation to the user. For example sensors associated with the user may collect certain data pertaining to the user, and a particular pathway may configure the computing device to receive data recorded by a particular sensing device associated with the user. The pathway may also include a specification that includes an expected value of the defined data points, and a trigger condition that uses a comparison of a value of the data point to be monitored with the expected value to define a triggering condition. In some embodiments, the computing device may execute a second pathway defining a second set of defined data points.

The computing device may then monitor the defined data points in relation to the customer (BLOCK 1610). The computing device (via, e.g., the network interface) can receive data collected by the sensors. In some arrangements, the computing device may be configured to compare any received data regarding the data points defined by the pathway with the expected value so as to determine if the triggering condition has occurred.

The computing device may then determine, by the pathway, responsive to monitoring values of the defined set of data points, a point in execution in which to communicate to the user (BLOCK 1615). In some embodiments, the point of execution is determined based on the triggering condition occurring. When the data measured by the sensors and received by the computing device differs from the expected value of the defined data points such that the triggering condition is met, the computer may establish a point of execution in which the user will be communicated with. In some arrangements, point of execution is determined responsive to executing the second pathway. For example, a point of execution may be determined by monitoring the second set of defined data points associated with the second pathway being executed by the computing device. The second pathway may include an indicator specifying a point of execution in which to communicate to the user. In various arrangements, pathways being executed by the computing devices may include a communication schedule, which may specify a time at which communication with the user is to be initiated.

The computing device may then select a communication scheme for communicating to the user (BLOCK 1620). In some embodiments, the pathway being executed may include a plurality of different communication schemes. These communication schemes may specify a mode by which the user is to be communicated with. For example, one communication scheme may specify that the user is to be connected with a health care navigator via a telephone call or text message to one or more telephone number associated with the user (stored, for example, in the storage unit of the computing device). Another communication schedule may specify the user is to receive an alert message taking the form an interface presented to the user on an application run by a smart device associated with the user. Other communication schedules may specify that the user is to be communicated with by a chat session run by a web-based application, or a video conference, among others.

In some arrangements, the platform may be configured to select from among these plurality of communication schemes based on the point of execution. For example, if the data collected from the sensors caused the triggering condition to be met, the computing device may be configured to select the communication scheme based on the data received from the sensors. If the received data differs from an expected value by more than a predetermined amount, for example, the computing device may select an alert message to be delivered to the user, taking the form of either a text message or a push notification on an application run by a user smart device. In some arrangements, if the data received by the sensors is indicative of a particularly serious measurement condition (i.e., the received data differs from the expected data by an abnormal amount), the computing device may select a communication scheme that calls for direct communication between the health navigator and the user, by way of a phone call, video chat, among others.

After the communication scheme is selected, the computing device is then configured to initiate the selected communication scheme (BLOCK 1625). In some embodiments, the computing device may prompt a health navigator to contact the user in conformance with the selected communication scheme. The computing device may transmit a message to a phone or smart device associated with the user, among others.

### E. Systems and Methods for Providing a Web Interface Monitoring Status of Pathways

By providing web interfaces for monitoring the status of pathways, users and health navigators may be able to communicate alerts, diagnoses, and health treatment plans, among others. In some embodiments, the system provides functional and interactive features to customers (e.g., in a mobile app at a smartphone). The system may provide a user experience and user interface, a design, a business process of data movement to allow for upstream and proactive health care, among others. In some embodiments, the customers may utilize the system via a user interface to provide health-related inputs (e.g., via sensors) to the system for analysis by the system, and the system may provide continuous health risk assessments based on the data inputs (e.g., from the sensors).

The web interface may be presented to a health navigator on a display device associated with a computing device. The computing device may include a first display device and a second display device. A first user interface may, via the first display device, display the first user interface displaying a plurality of tiles. In some implementations, a tile can be a computer representation of a user interface. The plurality of tiles may be arranged in any pattern on the first display device. In some embodiments, the first tiles are arranged, in a series of columns, with each column containing at least one tile. Each tile of the plurality of tiles may correspond to a user currently being monitored via one or more executing pathways. Each tile may identify the user, a status of alert in a pathway, and one or more last activities taken with respect to the user. Each tile may be selectable by the health care navigator through the manipulation of an input device associated with the computing device. The web interface may also present the health care navigator with a second user interface. In some embodiments, the second display device, may display the second user interface. The second interface may include a first queue of encounters with users currently in progress and a second queue of recently completed user encounters. In some embodiments, the first queue and the second queue may be arranged as lists under a first header and a second header, with the first header labelling the encounters in the first queue as being in progress and the second header labelling the encounters in the second queue as being complete.

By manipulating the input device, a health care navigator can make a first selection of a tile shown in the first user interface. By making the first selection, the health care navigator can initiate a first encounter with a first user. During the initiation of the first encounter, the health care navigator may be brought to an account preview interface. The encounter preview interface may include at least one of a participant list, a communication scheme selector for establishing communications between the user and a navigator, a first message from the user, and a second message from a navigator.

By further manipulating the input device on the second user interface, the health care navigator may make a second selection of a second user in the first queue to open a second encounter in progress with the second user. The second encounter may enable the navigator to take similar actions as in the first encounter, but includes information regarding actions already taken in the encounter, such as previous communications to the user, previous actions of the user, among others.

By making a further manipulation of the input device on the second user interface, the health care navigator can make a third selection of a third user in the second queue to view a third encounter completed with the third user. The view of the third encounter provides the navigators with details regarding aspects of the third encounter, such as communications to and from the customer, or any actions taken regarding the health condition of the user. In some embodiments, the third encounter completed with the third user further comprises displaying an encounter completion interface. The encounter completion interface may include at least one of a communication status indicator for communications completed between the user and a navigator, a health status of the user, an assent indicator for a treatment plan for the user, a first message from the user, and a second message from the navigator.

In some arrangements, the first, second, and third selections may be viewable simultaneously by the navigator on a separate interface. In other arrangements, the first, second, and third selections are viewable in sequence on additional separate interfaces. In some embodiments, the second encounter with the second user may include displaying an encounter progress interface. The encounter progress interface may include at least one of a communication scheme indicator for communications established between the user and a navigator, an urgency selector, a health status of the user, and an assent selector for a treatment plan.

In some embodiments, each tile of the plurality tiles may comprise at least one of a name, a contact information, an encounter availability status in the pathway, a health history of the user, a health status of the user, and a treatment plan. In some arrangements, the health history of the user displays information pertaining to pathway status of the user. In some embodiments, the first queue of encounters may further comprise at least one of a first profile picture of the user, a first login time, and a status of progress. The second queue of encounters further may include at least one of a second profile picture of the user, a second login time, and a status of completion.

In some embodiments, the device may determine a status of the alert in the pathway based on a defined set of data points from the pathway. In some embodiments, displaying the first user interface may further include modifying the first user interface, responsive to determining the status of the alert in the pathway. In some embodiments, displaying the second user interface may further include modifying the second user interface, responsive to determining the status of the alert in the pathway.

In some embodiments, the device may determine a status of the alert in the pathway based on a defined set of data points from a second pathway different from the pathway. In some embodiments, displaying the first user interface may further include modifying the first user interface, responsive to determining the status of the alert in the pathway from the defined set from the second pathway. In some embodiments, displaying the first user interface may further include modifying the second user interface, responsive to determining the status of the alert in the pathway from the defined set from the second pathway. In some embodiments, the device may display an edit menu for the user, the edit menu including at least one of a name, a contact information, a health history of the user, a health status of the user, and a treatment plan.

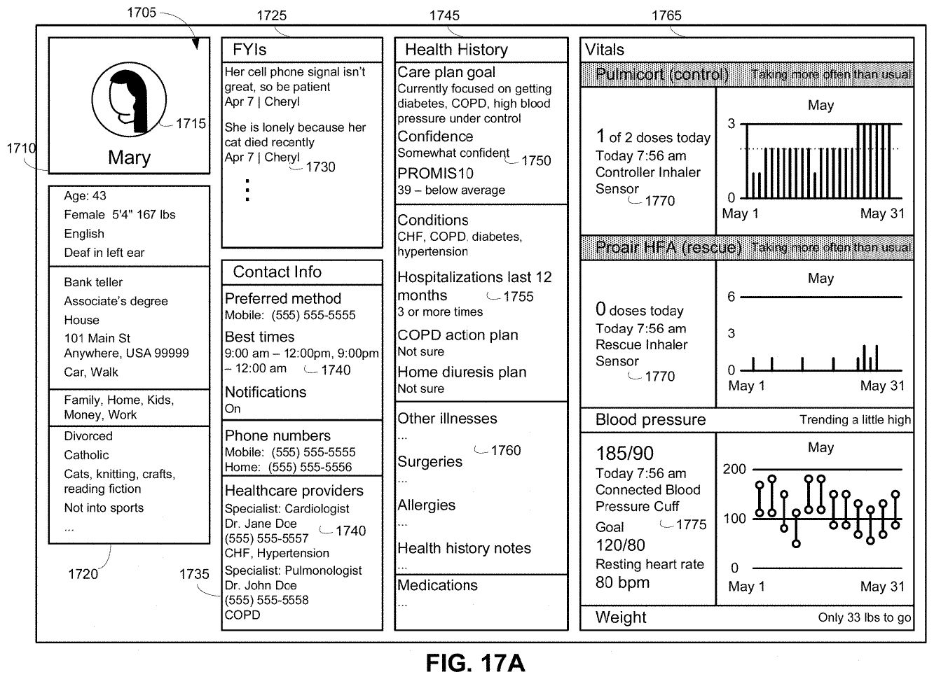

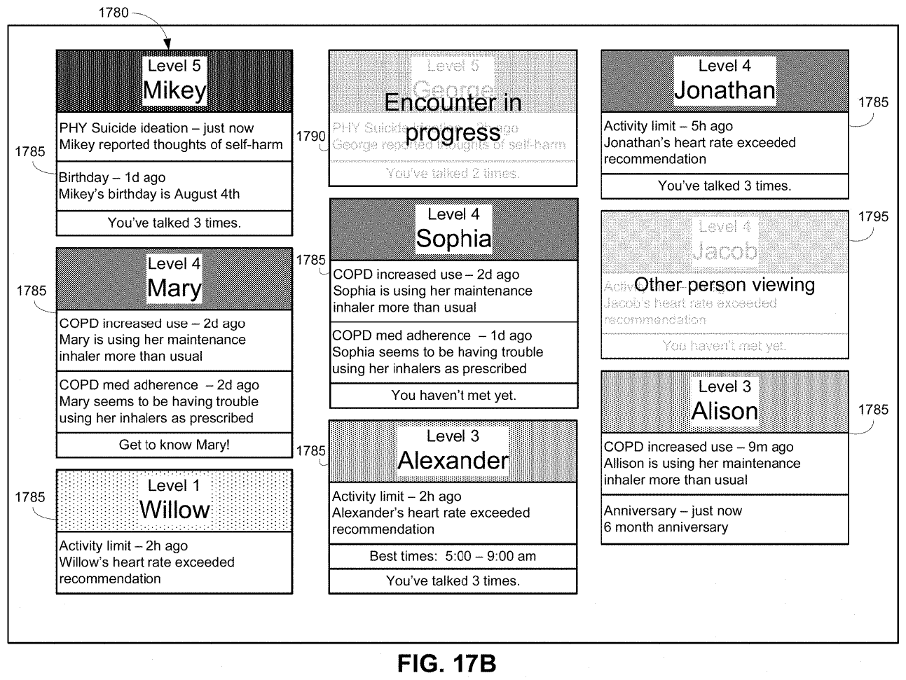

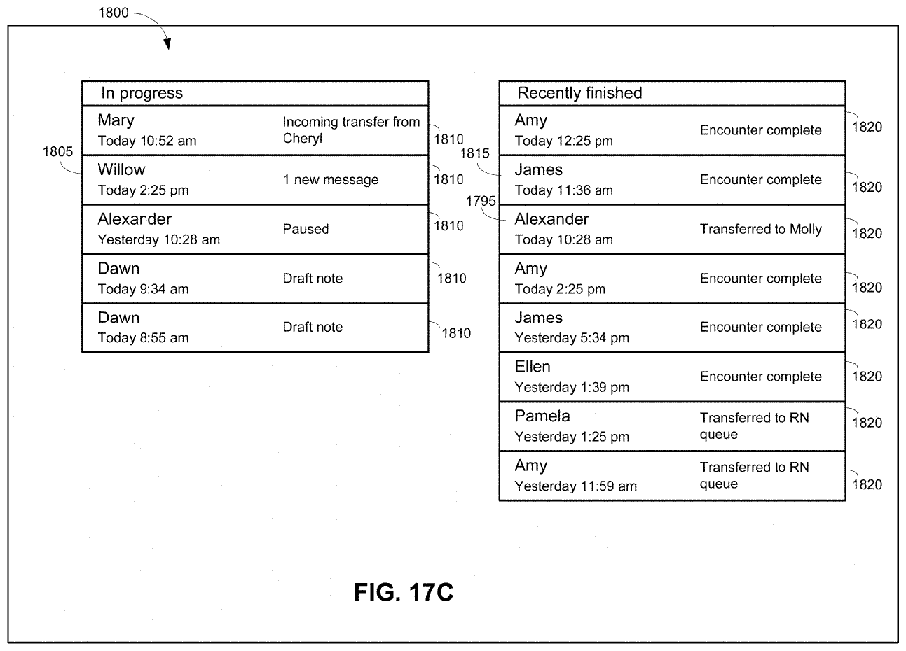

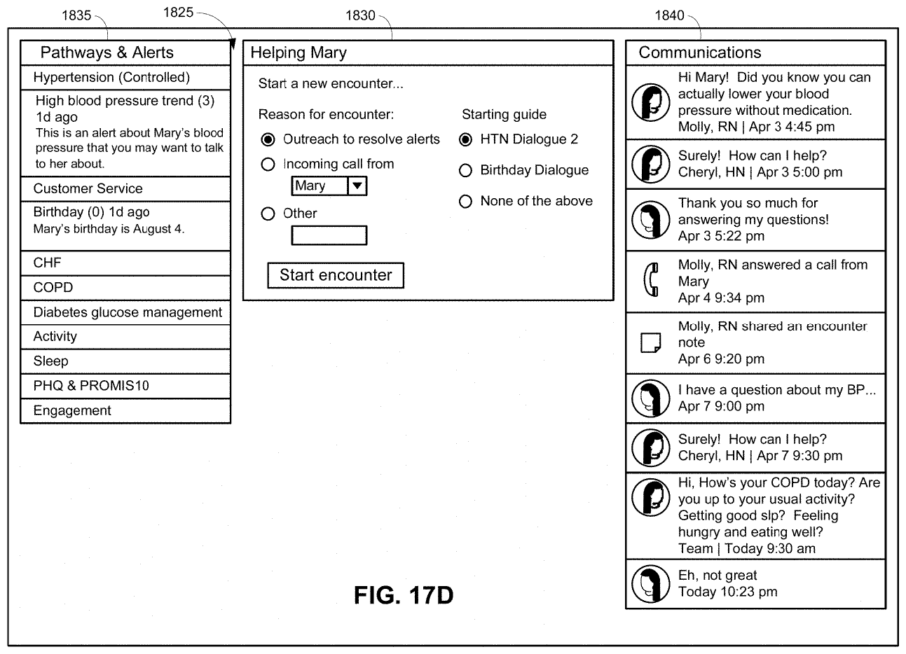

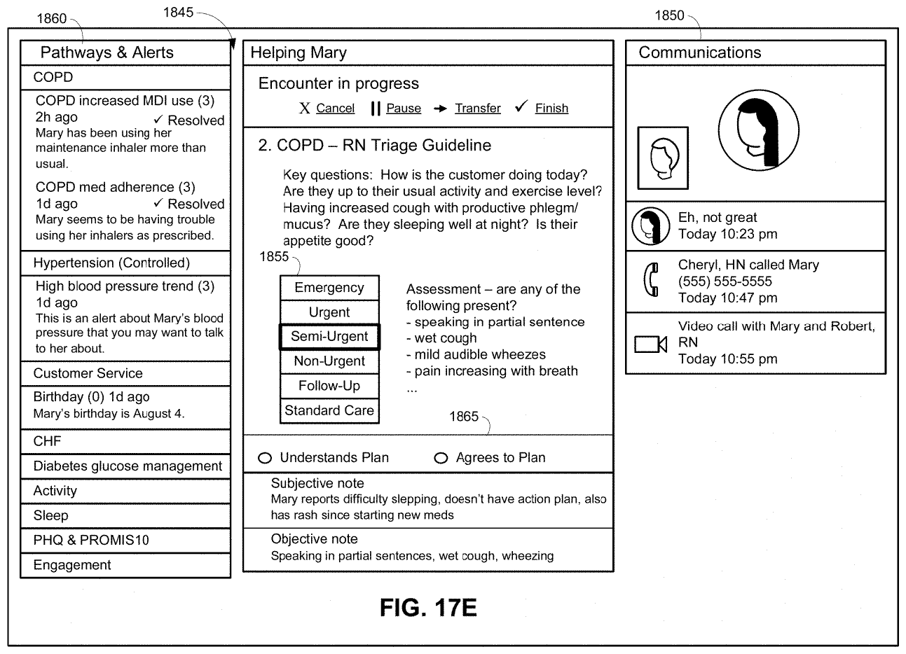

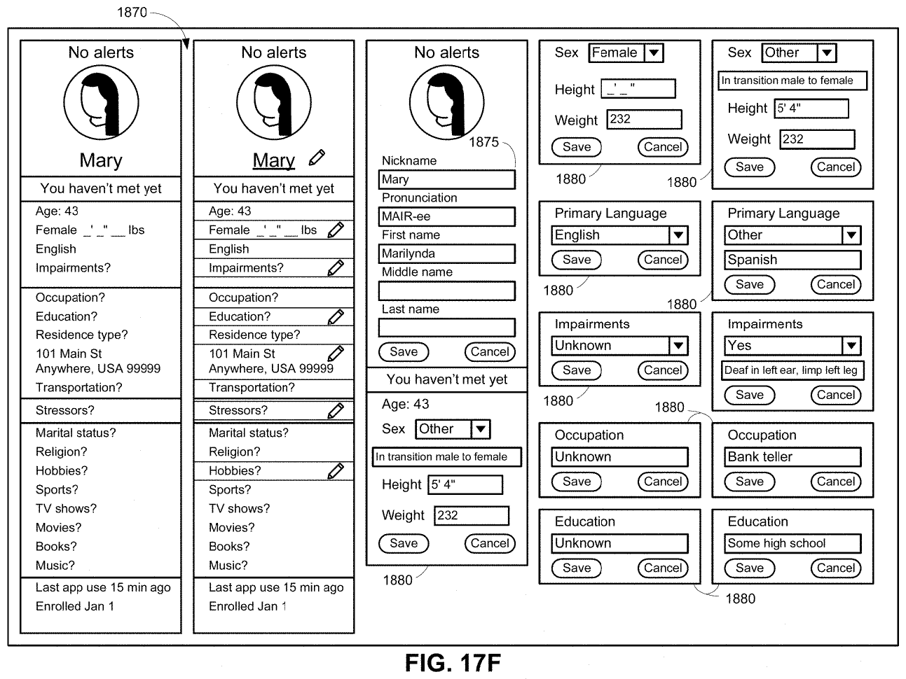

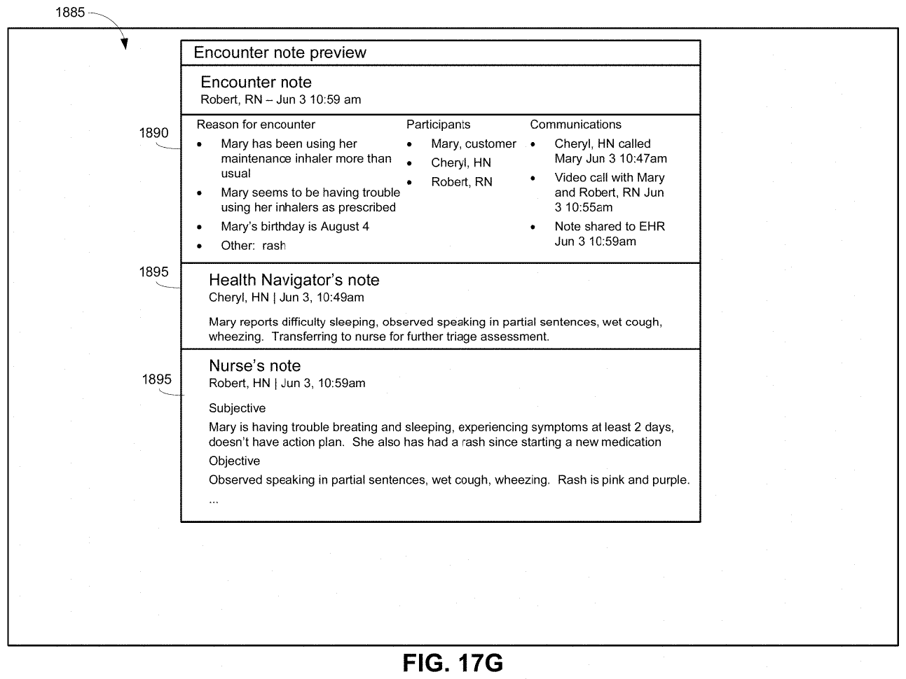

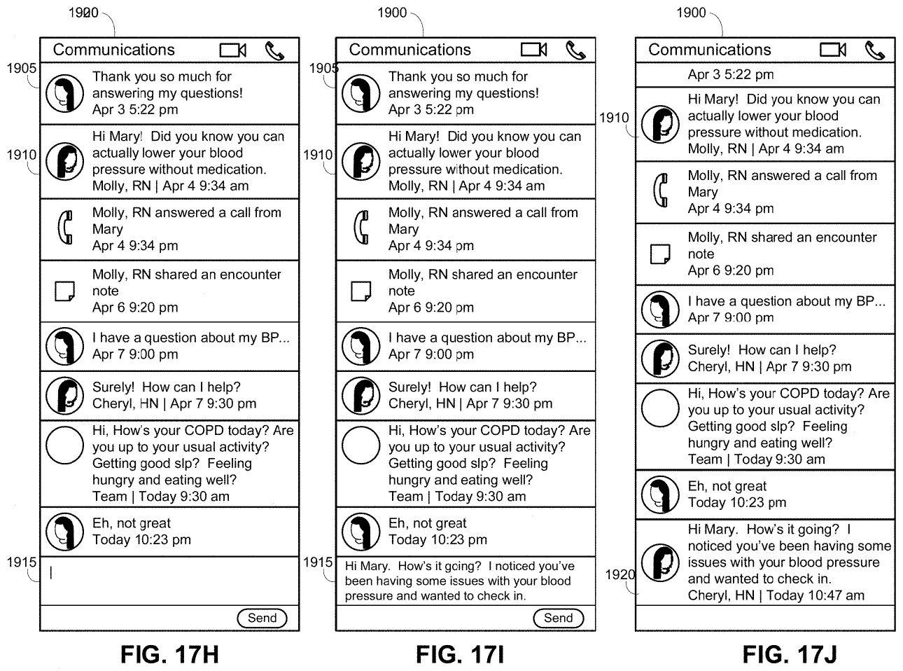

Referring to FIG. 17A, a profile interface 1705 is shown according to an illustrative embodiment. The profile interface 1705 may be shown by the display devices 124a-124n of the computing device 100 discussed above in relation to FIG. 1C. As shown, the interface 1705 may include a profile window 1710, a useful information window 1725, a contact information window 1735, a health history window 1745, and a vitals window 1765. The profile window 1710 may include a profile picture 1715 of the user as well as other user bibliographic information 1720. The other bibliographic information 1720 may include information pertaining to the age, gender, occupation, address, family status, and other information pertaining to the user. The useful information window 1725 includes additional information 1730 pertaining to the user. This additional information 1730 may have been input by other personnel (e.g., nurses, navigators, etc.) during past encounters with the user, and may identify user feelings, or describe the way that the user communicates. The contact information window 1735 contains customer contact information 1740, such as customer phone numbers, contact times, customer health care providers, among others. The health history window 1745 includes health care plan information 1750, a health condition summary 1755, and medical history information 1760. The health care plan information 1750 includes information that describes goals of the user, and a confidence that the user will achieve those goals. The health condition summary 1755 may describe current conditions that the user is suffering from, recent user hospital plans, among others. The medical history information 1760 contains a detailed record of a medical history of the user. This detailed record may describe other illnesses that the user has suffered from, user surgeries, user allergies, and other notes.

The vitals window 1765 includes user dosage information 1770, as well as user health monitoring information 1772. The user dosage information 1770 may contain information pertaining to the number and timing of any doses of any medication that the user has recently taken. The user health monitoring information 1772 may display recent measurements taken concerning various aspects of user health by various sensors, such as blood pressure, weight, heart rate, among others.

Referring now to FIG. 17B, a web interface 1800 is shown according to an illustrative embodiment. The web interface may be shown by the display devices 124a-124n of the computing device 100 discussed above in relation to FIG. 1C. As shown, the interface 1800 may include a plurality of tiles 1800 with each of the tiles corresponding to a different user. Each tile may include a user name, identifying the user associated with the tile 1800 as well user contact information. In some arrangements, the tiles 1800 may also include an encounter availability status, indicating times that the user may be available for an encounter with a health navigator or nurse, for example. In some arrangements, the tiles 1800 may also include information describing a user health history, describing various conditions and treatments that the user has had. In some arrangements, the tiles 1800 may also include a health status indicator for the customer, indicating how the user is coping with conditions that they suffer from, or any side effects of any current treatments. In some arrangements, the tiles 1800 may also include information pertaining to a treatment plan for the user, describing, for example, the timing and dosage for various treatments that the user is scheduled to undergo. In some embodiments, each tile 1800 is selectable and is configured such that, if selected (e.g., by the manipulation of an input device), an encounter with the user corresponding to the selected tile 1800 is initiated, and an encounter initiation interface may be displayed, as will be described in greater detail below. In some embodiments, responsive to an encounter being initiated, the first interface 1800 may update the selected tile 1782 to indicate that an encounter is in progress. In some embodiments, if a particular tile 1885 is selected by a person other than the person viewing the first web interface 1780, the tile is shown as an updated tile 1795 reflecting that the other person is viewing the tile.

Referring now to FIG. 17C, a second web interface 1800 is shown according to an illustrative embodiment. The web interface may be shown by the display devices 124a-124n of the computing device 100 discussed above in relation to FIG. 1C. In some arrangements, the web interface 1800 discussed above is displayed by a first display device of the computing device 100 at the same time that the second web interface 1800 is shown by a second display device of the computing device 100. As shown, the web interface 1800 may include a first queue 1805 and a second queue 1815. The first queue 1805 displays encounters 1810 with users that are currently in progress, or encounters with users that are yet to be complete. In some arrangements, each encounter 1810 displays a profile picture of the user associated with the incomplete encounter 1810, a first login time, describing the timing of the last time that progress was made on the encounter 1810, as well as a progress indicator, indicating that the encounter is still in progress. In some embodiments, each of the encounters 1810 in the first queue 1805 are selectable (e.g., by the manipulation of an input device). In some arrangements, if a particular encounter 1805 is selected by, for example, a health navigator, then the health navigator may be brought to an encounter progress interface that will be described in greater detail below.

The second queue 1815 displays a list of completed encounters 1820. In some embodiments, the list of completed encounters 1820 includes a user profile picture associated with each encounter 1820, a second login time, indicating the timing that the encounter was completed, as well as a completion status, indicating that the particular encounter was completed. In some arrangements, each of the encounters 1820 in the second queue 1800 are selectable (e.g., by the manipulation of an input device). In some arrangements, if a particular encounter 1820 is selected by, for example, a health navigator, then the health navigator may be brought to an encounter completion interface that will be described in greater detail below.

Referring now to FIG. 17D, an encounter initiation interface 1825 is shown according to an illustrative embodiment. The encounter initiation interface 1825 may be displayed by the first display device responsive to one of the tiles 1785 being selected in the first web interface 1780 discussed above in relation to FIG. 17B. As shown, encounter initiation interface 1825 includes a communication scheme selector 1830, a health status 1835, and a communications log 1840. A communication scheme selector 1830 enables, for example, a health navigator to select a type of communication that the encounter with the user is going to take. As shown, the communication scheme selector 1830 includes a list including a plurality of selectable options, enabling different types of dialogues to be started with the user. In other arrangements, the communication scheme selector 1830 may enable the health navigator to select from a plurality of communication forms, such as a phone call, text message, video conference, or graphical interface to present to the user via a mobile application. The health status 1835 may display information pertaining to a monitored health issue of the user. For example, in some arrangements, any pathways being executed with respect to the user may monitor a defined set of data points for the user by comparing any received user data with an expected value for the data points. The health status 1835 may reflect recent data points received with respect to the defined set of data points and give a description of any trends in the received data. In some arrangement, the health status 1835 may include alerts, flagging any issues that may be discussed in the encounter. The communications log 1840 includes communications between the users and various participants in the encounter. The communications log 1840 may include, for example, messages sent to and from the user, notes of phone calls made to the user, and any notes entered by encountered participants. In some arrangements, the encounter initiation interface 1825 may also include an urgency selector, which may similar to the communication scheme selector 1830 discussed above, and may include a list of selectable options, giving a health navigator the ability to indicate the urgency of the initiated encounter.

Referring now to FIG. 17E, an encounter progress interface 1845 is shown according to an illustrative embodiment. The encounter progress interface 1845 may be displayed by the second display device responsive to one of the encounters 1810 in the first queue 1805 being selected in the second web interface 1800 discussed above in relation to FIG. 17C. The encounter progress interface 1845 may identify a partially completed encounter with the user. As shown, the encounter progress interface 1845 includes a communication scheme indicator 1850, an urgency selector 1855, a health status 1860, and an assent selector 1865. The communication scheme indicator 1850 identifies previous communications established between the user and a health navigator. As shown, the communication scheme identifier 1850 identifies communications having multiple forms (e.g., a chat session message response from the user, a phone call from the health navigator, and a video call between the user and a registered nurse). The urgency selector 1855 provides the ability to indicate the urgency of the impending encounter with the user. As shown, the urgency selector 1855 includes a scale of selectable options, ranging from non-urgent to emergency, depending on symptoms shown by the user. The urgency selector 1855, responsive to a particular urgency level being selected, may be configured to present various symptoms that are associated with that urgency level. The health status 1860 of the encounter progress interface 1825 may include similar information as the health status 1835 associated with the encounter initiation interface 1825 discussed above. The assent selector 1865 gives the ability to indicate a user understanding and agreement to an identified plan of care. During the encounter, for example, the navigator may provide details of a plan of care for the user, and ask whether the user understands the plan of care and agrees to follow it. Responsive to the user answering these inquiries in the affirmative, the navigator may select the options in the health indicator 1865 to indicate the customer understanding and assenting to the plan of care.

In some embodiments, where, for example, a navigator selects one of the encounters 1820 in the second queue 1815 of the second interface 1800 discussed above in relation to FIG. 17C, an encounter completion interface (not shown) may be shown. The encounter completion interface may generally include information that is similar to the encounter progress interface 1845 discussed above, except that it may be updated to reflect the fact that there are no communications with the user in progress. Accordingly, in some embodiments, the encounter completion interface may also include a message from the user as well as a message from the navigator.

Referring now to FIG. 17F, an edit menu 1870 is shown according to an illustrative embodiment. In some embodiments the edit menu 1870 is transmitted to a smart device or a computing device associated with the user. In some arrangements, any information input into the edit menu 1870 may be used to populate the profile interface 1705 discussed above in relation to FIG. 17A. As shown, the edit menu 1870 provides the user with a name input 1875, and biographical inputs 1880, enabling the user to input information pertaining to age, occupation, language contact information, and user health information, among others.

Referring now to FIG. 17G, a note preview interface 1885 is shown according to an illustrative embodiment. In some arrangements, the note preview interface 1885 may preview a note that is to be entered into an encounter by an encounter participant. Encounter notes may be displayed, for example, in an encounter progress interface, such as the encounter progress interface 1840 discussed above in relation to FIG. 17E. As shown, the note preview interface 1885 includes an encounter summary window 1890 and a note windows 1895. The summary window 1890 may include information that summarizes the reasons for the encounter, the participants of the encounter, and any communications that took place in the encounter. The note windows 1895 may include notes that were taken by various participants (e.g., a navigator and a registered nurse) in the encounter regarding various actions taken during the encounter.

Referring now to FIG. 17H, a user chat interface 1900 is shown according to an illustrative embodiment. In some embodiments, encounters with the user may involve a navigator having an online chat with the user regarding user health issues. As shown, the chat interface 1900 shows a first message 1905 from the user, a second message 1910 from a registered nurse, and a description of other occurrences taking place within the encounter, such as phone calls and encounter notes entered by encounter participants. The chat interface 1900 also includes a message input window 1915, enabling an encounter participant to input a message that will be communicated to the user. As shown in FIG. 17I, a health navigator may enter a message into the message input window 1915 that is responsive to a message received from the user into the chat interface 1900. In some embodiments, as is shown in FIG. 17J, the message input by the encounter participant will update the chat interface 1900 so as to include a third message 1920 from a health navigator.

While this specification contains many specific implementation details, these should not be construed as limitations on the scope of any inventions or of what can be claimed, but rather as descriptions of features specific to particular embodiments of particular aspects. Certain features described in this specification in the context of separate embodiments can also be implemented in combination in a single embodiment. Conversely, various features described in the context of a single embodiment can also be implemented in multiple embodiments separately or in any suitable subcombination. Moreover, although features can be described above as acting in certain combinations and even initially claimed as such, one or more features from a claimed combination can in some cases be excised from the combination, and the claimed combination can be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing can be advantageous. Moreover, the separation of various system components in the embodiments described above should not be understood as requiring such separation in all embodiments, and it should be understood that the described program components and systems can generally be integrated in a single software product or packaged into multiple software products.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’ or only ‘B’, as well as both ‘A’ and ‘B’.

Thus, particular embodiments of the subject matter have been described. In some cases, the actions recited in the claims can be performed in a different order and still achieve desirable results. In addition, the processes depicted in the accompanying figures do not necessarily require the particular order shown, or sequential order, to achieve desirable results.

What is claimed is:

1. A method for providing a pathway configurable and executable via a pathway engine on a device, the method comprising:
(a) establishing, on a device, a pathway configured via a pathway engine to execute on the device to monitor one or more data points of a user and to generate one or more actions based on monitoring of the one or more data points;
(b) receiving, by the device, a specification for the pathway of a data point to be monitored, an expected value of the data point, a trigger condition using a comparison of a value of the data point to be monitored with the expected value to a predetermined threshold, and an alert and an action to take as a result of triggering the trigger condition;
(c) monitoring, by a monitor of the pathway engine, a plurality of data points of a user received by the device;
(d) comparing, by the monitor, each value of the plurality of data points to the expected value and the predetermined threshold specified for the trigger condition;
(e) determining, by the pathway based on the comparison, that the trigger condition has been triggered; and
(f) initiating, by the pathway responsive to the triggering of the trigger condition, an alert to identify the trigger condition and execution of the action specified by the pathway.

2. The method of claim 1, wherein (e) further comprises determining that a difference between the value of the data point and the expected value is greater than the predetermined threshold.

3. The method of claim 1, wherein (c) further comprises monitoring the data point by receiving data from a sensor measuring the value of the data point.

4. The method of claim 1, wherein the action comprises actuating an actuator coupled to the user.

5. The method of claim 1, comprising adjusting the expected value of the data point based on the action, responsive to executing the action.

6. The method of claim 1, comprising adjusting an expected value of a second data point of the pathway based on the action, the second data point of the user identified in the specification for the pathway as dependent on the first data point and the action, responsive to executing the action.

7. The method of claim 1, wherein executing the action comprises selecting, from a plurality of candidate actions, the action for execution.

8. The method of claim 7, wherein selecting, from the plurality of candidate actions, the action for execution further comprises:
determining, from a client device of the user, contextual information pertaining to the user via one or more monitors of the pathway engine; and
selecting, using the contextual information, the action for execution from the plurality of candidate actions.

9. The method of claim 8, wherein the contextual information include location information from the client device of the user.

10. The method of claim 8, wherein contextual information includes a plurality of physiological measurements.

11. A method for providing a multi-level pathway configurable and executable via a pathway engine on a device, the method comprising:
(a) establishing, on a device, a pathway configured via a pathway engine to execute on the device to monitor one or more data points of a user and trigger cascading levels of actions based on monitoring of the one or more data points;
(b) receiving, by the device, a specification for the pathway of a data point to be monitored, an expected value of the data point, a trigger condition using a comparison of a value of the data point to be monitored with the expected value to a predetermined threshold, and a first level alert and a first action to take as a result of triggering the triggering condition;
(c) receiving, by the device, for configuration of the pathway, one of a condition of the user or frequency of triggering the trigger condition upon which to trigger a second level alert and a second action to take subsequent to trigger of the first level alert and the first action;
(d) comparing, by a monitor of the pathway engine, values of a plurality of data points received by the device to the expected value and the predetermined threshold specified for the trigger condition;
(e) initiating, by the pathway, responsive to the triggering of the trigger condition, the first level alert to identify the trigger condition and execution of the first action specified by the pathway; and
(f) monitoring, by the monitor of the pathway engine, responsive to triggering of the trigger condition, values of the plurality of data points for one of the condition of the user or frequency of triggering the trigger condition upon which to trigger the second level alert and the second action.

12. The method of claim 11, wherein (e) further comprises determining that a difference between the value of the data point and the expected value is greater than the predetermined threshold.

13. The method of claim 11, wherein (c) further comprises monitoring the data point by receiving data from a sensor measuring the value of the data point.

14. The method of claim 11, wherein the first action comprises actuating an actuator coupled to the user and wherein the second action comprises actuating the actuator coupled to the user.

15. The method of claim 11, comprising adjusting the expected value of the data point based on the first action, responsive to executing the first action.

16. The method of claim 11, comprising adjusting an expected value of a second data point of the pathway based on the first action, the second data point of the user identified in the specification for the pathway as dependent on the first data point and the first action, responsive to executing the first action.

17. The method of claim 11, wherein executing the first action comprises selecting, from a plurality of candidate actions, the first action for execution.

18. The method of claim 17, wherein selecting, from the plurality of candidate actions, the first action for execution further comprises:
determining, from a client device of the user, contextual information pertaining to the user via one or more monitors of the pathway engine; and
selecting, using the contextual information, the first action for execution from the plurality of candidate actions.

19. The method of claim 18, wherein the contextual information include location information from the client device of the user.

20. The method of claim 18, wherein contextual information includes a plurality of physiological measurements.
